(19) United States
(12) Patent Application Publication
(10) Pub. No.: US 2018/0312561 A1
(43) Pub. Date: Nov. 1, 2018

(54) FOCUSED INTERFERON IMMUNOTHERAPY FOR TREATMENT OF CANCER

(71) Applicant: ImmunGene, Inc., Camarillo, CA (US)

(72) Inventors: Michael Gresser, Ojai, CA (US); Sanjay Khare, Palo Alto, CA (US); Kristopher Steward, Newbury Park, CA (US)

(21) Appl. No.: 15/735,583

(22) PCT Filed: Jun. 10, 2016
(86) PCT No.: PCT/US16/36925

§ 371 Date: Dec. 11, 2017

Related U.S. Application Data
(60) Provisional application No. 62/175,044, filed on Jun. 12, 2015.
(60) Provisional application No. 62/175,024, filed on Jun. 12, 2015.
(60) Provisional application No. 62/257,852, filed on Nov. 20, 2015.
(60) Provisional application No. 62/321,724, filed on Apr. 12, 2016.

(51) Int. Cl. C07K 14/56; C07K 16/32; C07K 14/565; C07K 16/28; C07K 16/18; A61K 45/06; A61K 38/21; A61K 39/395; A61P 35/00

(52) U.S. Cl. CPC C07K 14/56; A61K 2039/545; C07K 14/565; C07K 16/2887; C07K 16/2896; C07K 16/18; C07K 16/2803; C07K 16/2875; A61K 45/06; A61K 38/212; A61K 38/215; A61K 39/3955; A61K 39/39558; A61P 35/00; C07K 2319/00; C07K 16/32

(57) ABSTRACT

The present invention relates to methods of treating a proliferative disease (such as cancer) in an individual, comprising administering to the individual a non-naturally occurring fusion molecule comprising an antibody against one or more tumor-associated antigen (“TAA Ab”) attached to an interferon (IFN) molecule (hereinafter “TAA Ab-IFN fusion molecule”), as monotherapy at therapeutically effective low doses, or in combination with immunotherapy, wherein the combination therapy provides increased effector cell killing. The methods of the present invention are particularly effective treating recurrent, resistant, or refractory proliferative diseases.

### RELATED PATENT APPLICATIONS

This application claims benefit of U.S. Provisional Application No. 62/175,024, filed on Jun. 12, 2015, U.S. Provisional Application No. 62/175,044, filed on Jun. 12, 2015, U.S. Provisional Application No. 62/257,852, filed on Nov. 20, 2015, and U.S. Provisional Application No. 62/321,724, filed on Apr. 12, 2016, each incorporated in its entirety by reference herein.

### TECHNICAL FIELD

Cancer is group of diseases involving abnormal cell growth with the potential to spread or invade other parts of the body. Abnormal growths that form a discrete tumor mass, i.e., do not contain cysts or liquid areas, are defined as solid tumors. Solid tumors may be benign (not cancer), or malignant (cancer). Different types of solid tumors are named for the type of cells that form them. Examples of solid tumors are sarcomas, carcinomas, and lymphomas. Cancers derived from either of the two blood cell linages, myeloid and lymphoid, are defined as hematological malignancies. Such malignancies are also referred to as blood cancers or liquid tumors. Examples of liquid tumors include multiple myeloma, acute leukemias (e.g., 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (e.g., chronic myelocytic (granulocytic) leukemia, chronic myelogenous leukemia, and chronic lymphocytic leukemia), polycythemia vera, lymphoma, Hodgkin's lymphoma, non-Hodgkin's lymphoma (indolent and high grade forms), Waldenstrom's macroglobulinemia, heavy chain disease, myelodysplastic syndrome, hairy cell leukemia and myelodysplasia.

Interferons (IFNs) are soluble proteins produced naturally by cells in response to viruses. Although first described for their ability to inhibit viral replication, IFN-α's have multiple properties exhibiting anti-proliferative effects, induction of apoptosis (Rodriguez-Villanueva J and T J McDonnell, Int J Cancer, 61:110, 1995) and induction of the tumor suppressor gene, P53, in tumor cells (Takaoka A et al., Nature, 424:516, 2003). Thus, IFN-α's were the first recombinant proteins used for the treatment of various cancers. However, IFN-α as a single agent is largely ineffective at overcoming the numerous cellular mechanisms that mediate tumor cell resistance to proapoptotic agents. And unfortunately, the use of IFN-α to treat cancer has been limited by its short half-life and associated systemic toxicities (Weiss K, Semin Oncol, 25:9, 1998; Jones G J and Itri L M, Cancer, 57:1709, 2006). Given these limitations, it is difficult to achieve effective IFN-α concentrations at sites of malignant disease without causing systemic toxicity.

Cancer immunotherapy is the name given to cancer treatments that use the immune system to attack cancers. Systemic immunotherapy refers to immunotherapy that is used to treat the whole body and is more commonly used than local immunotherapy which is used to treat one “localized” part of the body, particularly when a cancer has spread. Although cancer cells are less immunogenic than pathogens, the immune system is clearly capable of recognizing and eliminating tumor cells, and cancer immunotherapy attempts to harness the exquisite power and specificity of the immune system for treatment of malignancy. Unfortunately, tumors frequently interfere with the development and function of immune responses, e.g., the suppressive milieu present within established tumors inhibits effective immune responses. The goal of immunotherapy is ultimately to re-establish immune system antitumor vigilance and to inhibit tumor and tumor-microenvironment immunosuppression. Thus, the challenge for immunotherapy is to use advances in cellular and molecular immunology to develop strategies which manipulates the local tumor environment to promote a pro-inflammatory environment, to promote dendritic cell activation, and to effectively and safely augment anti-tumor responses.

Cancer immunotherapy is enjoying a renaissance, and in the past few years the rapidly advancing field has produced several new methods of treating cancer. Numerous cancer immunotherapy strategies have been the focus of extensive research and clinical evaluation including, but not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab; treatment involving administration of biological response modifiers such as IL-2, IL-12, IL-15, IL-21, GM-CSF IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

Immunotherapy focused on utilization of depleting antibodies to specific tumor antigens have been explored with much success (see, e.g., reviews by Blattman and Greenberg, Science, 305:200, 2004; Adams and Weiner, Nat Biotech, 23:1147, 2005). A few examples of such tumor antigen-specific, depleting antibodies are HERCEPTIN® (anti-Her2/neu mAb) (Baselga et al., J Clin Oncology, Vol 14:737, 1996; Baselga et al., Cancer Research, 58:2825, 1998; Shak, Semin. Oncology, 26 (Suppl12):71, 1999; Vogal et al. J Clin Oncology, 20:719, 2002); and RITUXAN® (anti-CD20 mAb)(Colombat et al., Blood, 97:101, 2001). Unfortunately, while clearly having made a mark in oncology treatment, as monotherapy they generally work in only about 30% of the individuals and with a partial response. Moreover, many individuals eventually become refractory or relapse after treatment with these antibody-containing regimens.

Treatment using agonistic, antagonistic, or blocking antibodies to co-stimulatory or co-inhibitory molecules (immune checkpoints) has been an area of extensive research and clinical evaluation. Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll D M., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; pidilizumab) and PD-L1 (BMS-936559; MPLD3280A; MED14736; MSB0010718C) (see, e.g, Philips and Atkins, International Immunology, 27(1); 39-46, October 2014), and OX-40, CD137, GITR, LAGS, TIM-3, and VISTA (see, e.g., Sharon et al., Chin J Cancer, 33(9): 434-444, September 2014; Hodi et al., N Engl J Med, 2010; Topalian et al., N Engl J Med, 366:2443-54) are being evaluated as new, alternative immunotherapies to treat patients with proliferative diseases such as cancer, and in particular, patients with refractory and/or recurrent cancers.

Treatment using chimeric antigen receptor (CAR) T cell therapy is an immunotherapy in which the patient's own T cells are isolated in the laboratory, redirected with a synthetic receptor to recognize a particular antigen or protein, and reinfused into the patient. CARs are synthetic molecules that minimally contain: (1) an antigen-binding region, typically derived from an antibody, (2) a transmembrane domain to anchor the CAR into the T cells, and (3) 1 or more intracellular T cell signaling domains. A CAR redirects T cell specificity to an antigen in a human leukocyte antigen (HLA)-independent fashion, and overcomes issues related to T cell tolerance (Kalos M and June C H, Immunity, 39(1):49-60, 2013). Over the last 5 years, at least 15 clinical trials of CAR-T cell therapy have been published. A new wave of excitement surrounding CAR-T cell therapy began in August 2011, when investigators from the University of Pennsylvania (Penn) published a report on 3 patients with refractory chronic lymphocytic leukemia (CLL) who had long-lasting remissions after a single dose of CAR T cells directed to CD 19 (Porter D L, et al., N Engl J Med., 365(8):725-733, 2011).

In contrast to donor T cells, natural killer (NK) cells are known to mediate anti-cancer effects without the risk of inducing graft-versus-host disease (GvHD). Accordingly, alloreactive NK cells are now also the focus of considerable interest as suitable and powerful effector cells for cellular therapy of cancer. Several human NK cell lines have been established, e.g., NK-92, HANK-1, KHYG-1, NK-YS, NKG, YT, YTS, NKL and NK3.3 (Kornbluth, J., et al., J. Immunol. 134, 728-735, 1985; Cheng, M. et al., Front. Med. 6:56, 2012) and various CAR expressing NK cells (CAR-NK) have been generated. Immunotherapy using CAR expressing NK cells (CAR-NK) is an active area of research and clinical evaluation (see, e.g., Glienke et al., Front Pharmacol, 6(21):1-7, February 2015).

Bispecific T-cell engager molecules (BiTE®s) constitute a class of bispecific single-chain antibodies for the polyclonal activation and redirection of cytotoxic T cells against pathogenic target cells. BiTE®s are bispecific for a surface target antigen on cancer cells, and for CD3 on T cells. BiTE®s are capable of connecting any kind of cytotoxic T cell to a cancer cell, independently of T-cell receptor specificity, costimulation, or peptide antigen presentation. a unique set of properties that have not yet been reported for any other kind of bispecific antibody construct, namely extraordinary potency and efficacy against target cells at low T-cell numbers without the need for T-cell co-stimulation (Baeuerle et al., Cancer Res, 69(12):4941-4, 2009). BiTE antibodies have so far been constructed to more than 10 different target antigens, including CD19, EpCAM, Her2/neu, EGFR, CD66e (or CEA, CEACAM5), CD33, EphA2, and MCSP (or HMW-MAA) (Id.) Treatment using BiTE® antibodies such as blinatumomab (Nagorsen, D. et al., Leukemia & Lymphoma 50(6): 886-891, 2009) and solitomab (Amann et al., Journal of Immunotherapy 32(5): 452-464, 2009) are being clinically evaluated.

Despite the dramatic benefits and significant promise demonstrated by several of these immunotherapies, they remain limited by concerns over potential severe side effects and the fact that many tumors lack the targeted antigen and will therefore evade treatment. As such, there remains a critical need for new and improved immunotherapies to treat patients with proliferative diseases such as cancer, and in particular, patients with refractory and/or recurrent cancers.

### DISCLOSURE OF THE INVENTION

In one aspect, the present invention relates to combination therapies designed to treat a proliferative disease (such as cancer) in an individual, comprising administering to the individual: a) a tumor associated antigen antibody-interferon (“TAA Ab-IFN”) fusion molecule, and b) immunotherapy, wherein the combination therapy provides increased effector cell killing of tumor cells, i.e., a synergy exists between the TAA Ab-IFN fusion molecule and the immunotherapy when co-administered.

In various embodiments, the immunotherapy is selected from the group consisting of: treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF and IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod. In various embodiments, the immunotherapy is selected from the group consisting of: treatment using agonistic, antagonistic, or blocking antibodies to co-stimulatory or co-inhibitory molecules; treatment using chimeric antigen receptor (CAR)-T cells; treatment using CAR-NK cells; and treatment using bispecific T cell engaging antibodies (BiTE®). In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®).

In various embodiments, the fusion molecule comprises an TAA Ab selected from the group consisting of a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′₂, a IgG, a IgM, a IgA, a IgE, a scFv, a dsFv, a dAb, a nanobody, a unibody, and an diabody. In various embodiments, the antibody is a chimeric antibody. In various embodiments, the antibody is a humanized monoclonal antibody. In various embodiments, the antibody is a fully human monoclonal antibody. In various embodiments, the TAA Ab is a fully human antibody selected from the group consisting of a fully human anti-HER2/neu Ab, a fully human anti-CD20 Ab, a fully human anti-CD138 Ab, a fully human anti-GRP94 (endoplasmin) Ab, a fully human anti-CD33 Ab, and a fully human anti-CD70 Ab.

In various embodiments, the fusion molecule comprises a type 1 interferon molecule. In various embodiments, the fusion molecule comprises a type 1 interferon mutant molecule. In various embodiments, the fusion molecule comprises an interferon-alpha (IFN-α) molecule. In various embodiments, the fusion molecule comprises a human IFN-α2b molecule having the amino acid sequence of SEQ ID NO: 1. In various embodiments, the fusion molecule comprises a IFN-α2b mutant molecule having the amino acid sequence of SEQ ID NO: 2. In various embodiments, the fusion molecule comprises a human IFN-α14 molecule having the amino acid sequence of SEQ ID NO: 3. In various embodiments, the fusion molecule comprises an interferon-beta (IFN-β) molecule. In various embodiments, the fusion molecule comprises a human IFN-β-1a molecule having the amino acid sequence of SEQ ID NO: 4. In various embodiments, the fusion molecule comprises a human IFN-β-1b molecule having the amino acid sequence of SEQ ID NO: 5.

In various embodiments, the fusion molecules comprise an interferon molecule that is directly attached to the tumor associated antigen antibody.

In various embodiments, the fusion molecules comprise an IFN molecule that is attached to the TAA Ab via a peptide linker. In various embodiments, the peptide linker is fewer than 20 amino acids in length. In various embodiments, the peptide linker is a G/S rich linker. In various embodiments, the peptide linker is an alpha-helical linker. In various embodiments, the peptide linker has the sequence set forth in SEQ ID NO: 18. In various embodiments, the peptide linker has the sequence set forth in SEQ ID NO: 19.

In various embodiments, the fusion molecule is a recombinantly expressed fusion molecule.

In various embodiments, the proliferative disease is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In various embodiments, the individual previously responded to treatment with an anti-cancer therapy, but, upon cessation of therapy, suffered relapse (hereinafter “a recurrent cancer”). In various embodiments, the individual has a resistant or refractory cancer.

In various embodiments, there is provided a combination therapy method of treating a cancer selected from the group consisting of breast cancer, ovarian cancer and non-small cell lung cancer (NSCLC), comprising administering to the individual a) an effective amount of a pharmaceutical composition comprising an anti-HER2/neu-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the cancer expresses HER2/neu. In various embodiments, the cancer is a non-HER2/neu expressing cancer in the tumor microenvironment of a HER2/neu expressing cancer. In various embodiments, the immunotherapy will target a TAA that is different than HER2/neu.

In various embodiments, there is provided a combination therapy method of treating a cancer selected from the group consisting of B-cell Non-Hodgkin's lymphoma (NHL) and B-cell chronic lymphocytic leukemia (CLL), comprising administering to the individual a) an effective amount of a pharmaceutical composition comprising an anti-CD20 Ab-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the cancer expresses CD20. In various embodiments, the cancer is a non-CD20 expressing cancer in the tumor microenvironment of a CD20 expressing cancer. In various embodiments, the immunotherapy will target a TAA that is different than CD20.

In various embodiments, there is provided a combination therapy method of treating a cancer selected from the group consisting of multiple myeloma, breast cancer, and bladder cancer, comprising administering to the individual a) an effective amount of a pharmaceutical composition comprising an anti-CD138 Ab-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the cancer expresses CD138. In various embodiments, the cancer is a non-CD138 expressing cancer in the tumor microenvironment of a CD138 expressing cancer. In various embodiments, the immunotherapy will target a TAA that is different than CD138.

In various embodiments, there is provided a combination therapy method of treating a cancer selected from the group consisting of NSCLC, acute myeloid leukemia (AML), multiple myeloma, melanoma, and pancreatic cancer, comprising administering to the individual a) an effective amount of a pharmaceutical composition comprising an anti-GRP94 (endoplasmin) Ab-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the cancer expresses GRP94. In various embodiments, the cancer is a non-GRP94 expressing cancer in the tumor microenvironment of a GRP94 expressing cancer. In various embodiments, the immunotherapy will target a TAA that is different than GRP94.

In various embodiments, there is provided a combination therapy method of treating a cancer selected from the group consisting of AML, chronic myeloid leukemia (CML) and multiple myeloma, comprising administering to the individual a) an effective amount of a pharmaceutical composition comprising an anti-CD33 Ab-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the cancer expresses CD33. In various embodiments, the cancer is a non-CD33 expressing cancer in the tumor microenvironment of a CD33 expressing cancer. In various embodiments, the immunotherapy will target a TAA that is different than CD33.

In various embodiments, there is provided a combination therapy method of treating a cancer selected from the group consisting of renal cell carcinoma (RCC), Waldenstrom macroglobulinemia, multiple myeloma, and NHL, comprising administering to the individual a) an effective amount of a pharmaceutical composition comprising an anti-CD70 Ab-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the cancer expresses CD70. In various embodiments, the cancer is a non-CD70 expressing cancer in the tumor microenvironment of a CD70 expressing cancer. In various embodiments, the immunotherapy will target a TAA that is different than CD70.

In various embodiments, the combination therapy methods comprise administering the TAA Ab-IFN fusion molecule and immunotherapy simultaneously, either in the same pharmaceutical composition or in separate pharmaceutical compositions. Alternatively, the TAA Ab-IFN fusion molecule and immunotherapy are administered sequentially, i.e., the TAA Ab-IFN fusion molecule is administered either prior to or after the immunotherapy.

In various embodiments, the administration of the TAA Ab-IFN fusion molecule and immunotherapy are concurrent, i.e., the administration period of the TAA Ab-IFN fusion molecule and immunotherapy overlap with each other.

In various embodiments, the administrations of the TAA Ab-IFN fusion molecule and immunotherapy are non-concurrent. For example, in some embodiments, the administration of the TAA Ab-IFN fusion molecule is terminated before the immunotherapy is administered. In some embodiments, the administration of immunotherapy is terminated before the TAA Ab-IFN fusion molecule is administered.

In various embodiments, the methods may comprise one or more additional therapies selected from the group consisting of chemotherapy, small molecule kinase inhibitor targeted therapy, surgery, radiation therapy, and stem cell transplantation.

In another aspect, the present invention relates to a method of treating a proliferative disease in an individual, comprising administering to the individual a non-naturally occurring TAA Ab-IFN fusion molecule, wherein the TAA Ab-IFN fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the TAA Ab-IFN fusion molecule is administered to the individual at a weekly dosage selected from the group consisting of about 0.0001 mg/kg, of about 0.0003 mg/kg, of about 0.001 mg/kg, of about 0.003 mg/kg, of about 0.01 mg/kg, of about 0.03 mg/kg, of about 0.1 mg/kg, of about 0.2 mg/kg, of about 0.3 mg/kg, of about 0.4 mg/kg, of about 0.5 mg/kg, of about 0.6 mg/kg, of about 0.7 mg/kg, of about 0.8 mg/kg, and of about 0.9 mg/kg. In various embodiments, the TAA Ab-IFN fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses the TAA of the anti-TAA Ab-IFN-α fusion molecule of the present invention. In various embodiments, the cancer is a non-TAA expressing cancer in the tumor microenvironment of a TAA expressing cancer.

In various embodiments, the proliferative disease is a cancer selected from the group consisting of breast cancer, ovarian cancer and non-small cell lung cancer (NSCLC), comprising administering to the individual an effective amount of a pharmaceutical composition comprising an anti-HER2/neu-IFN-α fusion molecule, wherein the anti-HER2/neu-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses HER2/neu. In various embodiments, the cancer is a non-HER2/neu expressing cancer in the tumor microenvironment of a HER2/neu expressing cancer.

In various embodiments, the proliferative disease is a cancer selected from the group consisting of B-cell Non-Hodgkin's lymphoma (NHL) and B-cell chronic lymphocytic leukemia (CLL), comprising administering to the individual an effective amount of a pharmaceutical composition comprising an anti-CD20 Ab-IFN-α fusion molecule, wherein the anti-CD20-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses CD20. In various embodiments, the cancer is a non-CD20 expressing cancer in the tumor microenvironment of a CD20 expressing cancer.

In various embodiments, the proliferative disease is a cancer selected from the group consisting of multiple myeloma, breast cancer, and bladder cancer, comprising administering to the individual an effective amount of a pharmaceutical composition comprising an anti-CD138 Ab-IFN-α fusion molecule, wherein the anti-CD138-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses CD138. In various embodiments, the cancer is a non-CD138 expressing cancer in the tumor microenvironment of a CD138 expressing cancer.

In various embodiments, the proliferative disease is a cancer selected from the group consisting of NSCLC, acute myeloid leukemia (AML), multiple myeloma, melanoma, and pancreatic cancer, comprising administering to the individual an effective amount of a pharmaceutical composition comprising an anti-GRP94 (endoplasmin) Ab-IFN-α fusion molecule, wherein the anti-GRP94-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses GRP94. In various embodiments, the cancer is a non-GRP94 expressing cancer in the tumor microenvironment of a GRP94 expressing cancer.

In various embodiments, the proliferative disease is a cancer selected from the group consisting of AML, chronic myeloid leukemia (CML) and multiple myeloma, comprising administering to the individual a) an effective amount of a pharmaceutical composition comprising an anti-CD33 Ab-IFN-α fusion molecule, wherein the anti-CD33-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses CD33. In various embodiments, the cancer is a non-CD33 expressing cancer in the tumor microenvironment of a CD33 expressing cancer.

In various embodiments, the proliferative disease is a cancer selected from the group consisting of renal cell carcinoma (RCC), Waldenstrom macroglobulinemia, multiple myeloma, and NHL, comprising administering to the individual a) an effective amount of a pharmaceutical composition comprising an anti-CD70 Ab-IFN-α fusion molecule, wherein the anti-CD70-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses CD70. In various embodiments, the cancer is a non-CD70 expressing cancer in the tumor microenvironment of a CD70 expressing cancer.

In another aspect, the present invention provides a pharmaceutical composition which comprises a TAA Ab-IFN fusion molecule and a second anti-cancer agent as active ingredients, in a pharmaceutically acceptable excipient or carrier. In various embodiments, the pharmaceutical composition is formulated for administration via a route selected from the group consisting of subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

In other aspects, the present disclosure provides polynucleotides that encode the fusion molecules of the present disclosure; vectors comprising polynucleotides encoding fusion molecules of the disclosure; optionally, operably-linked to control sequences recognized by a host cell transformed with the vector; host cells comprising vectors comprising polynucleotides encoding fusion molecules of the disclosure; a process for producing a fusion molecule of the disclosure comprising culturing host cells comprising vectors comprising polynucleotides encoding fusion molecules of the disclosure such that the polynucleotide is expressed; and, optionally, recovering the fusion molecule from the host cell culture medium.

### BRIEF DESCRIPTION OF THE DRAWINGS

FIG. 1 is a schematic diagram of an exemplary TAA antibody-IFN fusion molecule.

FIG. 2 shows the ADCC and CDC activity of IGN002 as compared to the IGN002 non-fused mAb using Daudi (ATCC CCL-213) and Ramos (ATCC CRL-1596) NHL cell lines. For the ADCC experiment, Daudi NHL tumor cells were incubated with the indicated concentration of IGN002 fusion protein or IGN002 mAb for 15 minutes, then normal human peripheral blood mononuclear cells (PBMC) were added to the tumor cells as effector cells to achieve an effector to target cell ratio (E:T ratio) of 50:1. Plates were incubated overnight for 16 hours at 37° C. in a 5% CO₂ atmosphere. After incubation, standard lactate dehydrogenase (LDH) assay (Roche Diagnostics) was used to determine the % target cell lysis following manufacturer's instructions. Dose response curves were generated by non-linear regression analysis using Prism software. For the CDC experiment, Ramos NHL tumor cells were incubated with the indicated concentration of IGN002 fusion protein or IGN002 mAb for 15 minutes on ice, then normal human complement serum (Quidel) was added to each tube to achieve a final concentration of 10%. Samples were incubated at 37° C. in a 5% CO₂ atmosphere for 3 hours, then viability was assessed by propidium iodide (PI) flow cytometry following the manufacturer's instructions (Roche Diagnostics). Dose response curves were generated by non-linear regression analysis using Prism software. It was demonstrated that IGN002 demonstrated superior ADCC and CDC activity, compared to non-fused antibody.

FIG. 3 shows the STAT1 phosphorylation and proliferation inhibition activities of IGN004 compared to non-fused IFN-α2b in a non-targeted and a targeted setting. For the non-targeted STAT1 phosphorylation experiment, Daudi NHL tumor cells (GRP94-negative) were incubated with the indicated concentration of IGN004 or IFN-α2b for 15 minutes, then cells were fixed, permeabilized and intracellularly stained with PE-labeled anti-STAT1 (pY701) or PE-labeled isotype control. For the targeted proliferation inhibition experiment, GRP94-positive NCI-H1299 NSCLC tumor cells (ATCC CRL-5803) were treated with the indicated concentration of IGN004 fusion protein or IFN-α2b for 96 hours at 37° C. in a 5% CO₂ atmosphere. After incubation, standard MTS assay (Promega Cell Titer96; Promega, Madison, Wis.) was performed to assess cellular proliferation. Dose response curves were generated by non-linear regression analysis using Prism software.

FIG. 4 shows the in vivo anti-tumor efficacy of IGN004 in the U266 human multiple myeloma xenograft tumor model. Groups of 8 NOG immunodeficient mice bearing 11-day established subcutaneous U266 human multiple myeloma xenograft tumors were treated with vehicle (PBS) or IGN004 at 5, 1, or 0.2 mg/kg intravenously twice per week for 4 weeks. Tumors were measured bidirectionally using calipers and tumor volume calculated as 0.5×(L×W²). Animals were followed for survival and sacrificed when their tumors reached 2000 mm³. Average Tumor Volume (mm³) is plotted vs. Days Post Tumor Challenge.

FIG. 5 shows the tumor cell killing activity of the human CD8+ NKT cell-like TALL-104 effector cell line (ATCC CRL-11386) assessed in the presence or absence of IGN004 using the A549 human NSCLC tumor cell line (ATCC CCL-185). IGN004 treatment caused a small decrease in the viability of the A549 tumor cells (15.82%). TALL-104 effector cells demonstrated robust killing in the absence of IGN004 (69.2%). However, the combination of IGN004 and TALL-104 cells lead to complete eradication of A549 tumor cells (100% killing). This effect was stronger than the combination of either agent alone (85.02% vs. 100%).

FIG. 6 shows the tumor cell killing activity of TALL-104 effector cells assessed in the presence or absence of IGN004 at two different E:T ratios using a different human NSCLC tumor cell line (NCI-H1975; ATCC CRL-5908). IGN004 treatment caused a small decrease in the viability of the A549 tumor cells (5.7% and 10.6%). TALL-104 effector cells demonstrated significant killing in the absence of IGN004 and both 5:1 and 3.3:1 E:T ratios (58.6% and 55.7%, respectively). However, the combination of 50 pM IGN004 and TALL-104 cells lead to much more effective killing of the NCI-H1975 tumor cell targets at both E:T ratios (93.8% and 93.2%, respectively).

FIG. 7 shows the potency of the TALL-104 tumor cell killing assessed in the presence of IGN004 using NCI-H1975 NSCLC tumor cells. TALL-104 effector cells killed 17% of the NCI-H1975 tumor cells in the absence of IGN004 co-treatment. Treatment with IGN004 in combination with TALL-104 cells at concentrations from 0.25 to 25 pM caused an increase in tumor cell killing, compared to TALL-104 treatment alone.

FIG. 8 shows the tumor cell killing activity of downregulated TALL-104 effector cells assessed on A549 NSCLC tumor cells in the presence or absence of 10 pM IGN004 at different E:T ratios. 10 pM IGN004 alone had no effect on the tumor cells. At the 3:1 E:T ratio TALL-104 cells killed approximately 40% of the A549 tumor cells in the absence of drug but at lower E:T ratios the effector cells were ineffective at tumor cell killing. In the presence of 10 pM IGN004 the TALL-104 cells demonstrated robust tumor cell killing, even at 0.75:1 E:T where TALL-104 had no effect on the tumor cells without drug.

FIG. 9 shows the tumor cell killing activity of TALL-104 effector cells assessed in the presence or absence of IGN004 fusion protein or IGN004 non-fused mAb. 10 pM IGN004 non-fused mAb alone had no effect on the tumor cells and 10 pM IGN004 had only a slight effect (<10%). At all E:T ratios TALL-104 cells demonstrated a low level of tumor cell killing in the absence of drug. In the presence of 10 pM IGN004 mAb, the TALL-104 cells killed at an equivalent rate to TALL-104 cells without drug. However, with 10 pM IGN004 there was a significant increase in the tumor cell killing by TALL-104 cells, compared to no drug (70-80% vs. 10-20% killing).

FIG. 10 shows the tumor cell killing activity of TALL-104 effector cells assessed in the presence or absence of IGN004, a control TAA Ab-IFN-α fusion molecule, or the combination of IGN004 non-fused mAb+non-fused IFN-α. 10 pM control TAA Ab-IFN-α fusion molecule alone had no effect on the tumor cells. 10 pM IGN004 or the combination of IGN004 non-fused mAb and non-fused IFN-α2b had only a slight effect (<10%). At both E:T ratios TALL-104 cells demonstrated a low level of tumor cell killing in the absence of drug (<10%). In the presence of 10 pM control TAA Ab-IFN-α fusion molecule the TALL-104 cells killed at an equivalent rate to TALL-104 cells without drug. With 10 pM of the combination of IGN004 mAb+non-fused IFN-α2b the TALL-104 effector cells killed more A549 tumor cells (14% and 25% increase in killing at 1:1 and 1.5:1 E:T, respectively). However, with 10 pM IGN004 there was a much higher increase in the tumor cell killing by TALL-104 cells, compared to no drug (34% and 42% increase in killing at 1:1 and 1.5:1, respectively).

FIG. 11 shows the tumor cell killing activity of the NK effector cell line NK-92 (ATCC CRL-2407) assessed in the presence or absence of IGN004 or a control TAA Ab-IFN-α fusion molecule at two E:T ratios using the OVCAR-3 ovarian cancer cell line (ATCC HTB-161). 10 pM of either treatment protein had no effect on the tumor cells in the absence of effector cells. NK-92 effector cells demonstrated robust killing of tumor cells in the absence of drug at 1.5:1 E:T ratio (49% killing) and modest killing at 0.5:1 (19% killing). In the presence of 10 pM control TAA Ab-IFN-α fusion molecule the NK-92 cells killed at an equivalent rate to effector cells without drug. With 10 pM IGN004 there was a significant increase in the tumor cell killing by NK-92 cells, compared to no drug (45% and 29% increase in killing at 1.5:1 and 0.5:1, respectively).

FIG. 12 shows the tumor cell killing activity of the NK-92 effector cells assessed in the presence or absence of IGN004 or non-fused IFN-α2b at two E:T ratios using NCI-H1975 NSCLC tumor cells. Treatment with either protein had no effect on the tumor cells in the absence of effector cells. NK-92 effector cells demonstrated little to no killing of tumor cells in the absence of drug. In the presence of 100 pM non-fused IFN-α the NK-92 cells killed more tumor cells than NK-92 cells in the absence of drug. With 10 pM IGN004 there was a significant increase in the tumor cell killing by NK-92 cells, compared to no drug (85% and 62% increase in killing at 1:1 and 0.3:1, respectively) and non-fused IFN-α2b (50% and 51% increase in killing at 1:1 and 0.3:1, respectively).

### MODE(S) FOR CARRYING OUT THE DISCLOSURE

The present disclosure is based on the inventors' insight that a fusion molecule which combines the specificity of an antibody to the target antigen with the potent cytotoxic effects of the IFN molecule would significantly improve the efficacy and safety profiles of current cancer immunotherapies and/or IFN-based therapies, based, in part, on their understanding that use of the TAA Ab-IFN fusion molecule will have the following major advantages as compared with non-fused IFN: 1) the potent cytotoxic effects (induced apoptosis and programmed cell death) of IFN is concentrated at the targeted tumor cells by the fusion molecule (as compared with non-fused IFN) and engagement with IFN-αR expressed on the tumor cells will serve to eradicate the tumor cells; 2) the specificity of the TAA antibody to the target antigen will spar non-targeted cells, providing for a reduction of the systemic toxicity of IFN; 3) the local actions of IFN-α on dendritic cells (DCs) in the tumor microenvironment will help to negate some of the suppressive actions of tumors on DCs and could potentially lead to more efficient cross presentation of tumor antigens to T cells by DCs; 4) the IFN-α will act directly on T cells including enhancement of the CD8+ CTL functions, improving CD8+ CTL priming by increased DC cross-presentation which could potentially improve the therapeutic effect of the fusion protein; 5) the fusion molecule will stimulate or activate immune cells in lymphoid organs (e.g., draining lymph nodes, spleen, bone marrow); 6) the fusion molecule will stimulate or activate immune cells (e.g., T cells, natural killer cells, antigen presenting cells, phagocytic cells) that are present in the tumor microenvironment by directly binding to them via antibody-TAA and/or IFN-IFNαR interaction; 7) direct activation of the CD8+ CTL functions which will allow efficient killing of tumor cells; 8) inducing the up-regulation of the co-inhibitory immune-checkpoint proteins expressed on or associated with tumor cells; 9) inducing the up-regulation of MHC class I expressed on or associated with tumor cells, leading to better antigen presentation to T cells; and 10) directly negating other mechanisms for immune evasion, e.g., the major inhibitory pathways mediated by certain immune-checkpoint proteins on T cells/B cells.

As described herein, the inventors' found that 1) the TAA Ab-IFN fusion molecules can be used in combination with immunotherapy to design treatment protocols that provide for increased effector cell killing of tumor cells (i.e., a synergy exists between the TAA Ab-IFN fusion molecule and immunotherapy when co-administered); and 2) the TAA Ab-IFN fusion molecules and methods described herein can be used to effectively treat cancers, including recurrent, resistant, or refractory cancers, at surprisingly low doses. Specifically, the TAA Ab-IFN fusion molecules and methods described herein appear to be optimal for leveraging IFN's multiple properties and demonstrate the following: 1) effective killing of TAA-expressing tumor cells; and 2) the ability to provide for killing of non-TAA expressing tumor cells (also referred to hereinafter as “bystander tumor cells”) that are adjacent to or held in close proximity to the tumor cells that express the TAA (i.e., non-TAA expressing tumor cells located in the tumor microenvironment). These observed “bystander effects” on non-TAA expressing tumor cells are surprising, given that the fused IFN has much lower affinity for the IFN receptor than does non-fused IFN, and thus has much lower potency for stimulation of the INF receptor on the non-TAA expressing tumor cells, as compared to non-fused IFN. And, importantly, the apparent bystander effects are only observed on non-TAA expressing tumor cells in the tumor microenvironment, or when immune cells in the tumor microenvironment have been stimulated by any kind of immunotherapy designed to attack the TAA expressing tumor cells. The TAA Ab-IFN fusion molecules and methods of the present invention thus represent promising new effective therapies to treat patients with proliferative diseases, and in particular, patients with recurrent, resistant or refractory proliferative diseases.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of patients.

### Definitions

The term “tumor associated antigen” (TAA) refers to, e.g., cell surface antigens that are selectively expressed by cancer cells or over-expressed in cancer cells relative to most normal cells. The terms “TAA variant” and “TAA mutant” as used herein refers to a TAA that comprises an amino acid sequence wherein one or more amino acid residues are inserted into, deleted from and/or substituted into the amino acid sequence relative to another TAA sequence. In various embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length.

As used herein, the term “tumor microenvironment” refers to the cellular environment in which the tumor exists, including surrounding blood vessels, immune cells, fibroblasts, bone marrow-derived inflammatory cells, lymphocytes, signaling molecules and the extracellular matrix (ECM). Components in the tumor microenvironment can modulate the growth of tumor cells, e.g., their ability to progress and metastasize. The tumor microenvironment can also be influenced by the tumor releasing extracellular signals, promoting tumor angiogenesis and inducing peripheral immune tolerance.

As used herein, a “proliferative disease” includes tumor disease (including benign or cancerous) and/or any metastases. A proliferative disease may include hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In some embodiments, the proliferative disease is cancer. In some embodiments, the proliferative disease is a non-cancerous disease. In some embodiments, the proliferative disease is a benign or malignant tumor.

As used herein, “treatment” is an approach for obtaining beneficial or desired clinical results. For purposes of this invention, beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, and remission (whether partial or total). Also encompassed by “treatment” is a reduction of pathological consequence of a proliferative disease. The methods of the invention contemplate any one or more of these aspects of treatment.

As used herein, the term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAGS, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-β and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

“Enhancing T cell function” means to induce, cause or stimulate an effector or memory T cell to have a renewed, sustained or amplified biological function. Examples of enhancing T-cell function include: increased secretion of γ-interferon from CD8+ effector T cells, increased secretion of γ-interferon from CD4+ memory and/or effector T-cells, increased proliferation of CD4+ effector and/or memory T cells, increased proliferation of CD8+ effector T-cells, increased antigen responsiveness (e.g., clearance), relative to such levels before the intervention. The manner of measuring this enhancement is known to one of ordinary skill in the art.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to NHL and other cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

“Adjuvant setting” refers to a clinical setting in which an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated.

As used herein, the terms “co-administration”, “co-administered” and “in combination with”, referring to the fusion molecules of the invention and one or more other therapeutic agents, is intended to mean, and does refer to and include the following: simultaneous administration of such combination of fusion molecules of the invention and therapeutic agent(s) to an individual in need of treatment, when such components are formulated together into a single dosage form which releases said components at substantially the same time to said individual; substantially simultaneous administration of such combination of fusion molecules of the invention and therapeutic agent(s) to an individual in need of treatment, when such components are formulated apart from each other into separate dosage forms which are taken at substantially the same time by said individual, whereupon said components are released at substantially the same time to said individual; sequential administration of such combination of fusion molecules of the invention and therapeutic agent(s) to an individual in need of treatment, when such components are formulated apart from each other into separate dosage forms which are taken at consecutive times by said individual with a significant time interval between each administration, whereupon said components are released at substantially different times to said individual; and sequential administration of such combination of fusion molecules of the invention and therapeutic agent(s) to an individual in need of treatment, when such components are formulated together into a single dosage form which releases said components in a controlled manner whereupon they are concurrently, consecutively, and/or overlappingly released at the same and/or different times to said individual, where each part may be administered by either the same or a different route.

The term “therapeutic protein” refers to proteins, polypeptides, antibodies, peptides or fragments or variants thereof, having one or more therapeutic and/or biological activities. Therapeutic proteins encompassed by the invention include but are not limited to, proteins, polypeptides, peptides, antibodies, and biologics (the terms peptides, proteins, and polypeptides are used interchangeably herein). It is specifically contemplated that the term “therapeutic protein” encompasses the fusion molecules of the present invention.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

“Pharmaceutical composition” refers to a composition suitable for pharmaceutical use in a human. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

The phrase “administering” or “cause to be administered” refers to the actions taken by a medical professional (e.g., a physician), or a person controlling medical care of a patient, that control and/or permit the administration of the agent(s)/compound(s) at issue to the patient. Causing to be administered can involve diagnosis and/or determination of an appropriate therapeutic regimen, and/or prescribing particular agent(s)/compounds for a patient. Such prescribing can include, for example, drafting a prescription form, annotating a medical record, and the like. Where administration is described herein, “causing to be administered” is also contemplated.

“Resistant or refractory cancer” refers to tumor cells or cancer that do not respond to previous anti-cancer therapy including, e.g., chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

### Interferon and Interferon Mutants

In the fusion molecules of the present disclosure, either the N- or C-terminus of a TAA antibody, or antigen-binding fragment heavy or light chain will be genetically constructed with one of the several contemplated interferons or interferon mutants. Interferons include type I interferons (e.g., IFN-α, IFN-β) as well as type II interferons (e.g., IFN-γ). The term “interferon” as used herein refers to a full-length interferon or to an interferon fragment (truncated interferon) or to an interferon mutant (truncated interferon and interferon mutant collectively referred to herein as ‘modified interferon’), that substantially retains the biological activity of the full length wild-type interferon (e.g., retains at least 50%, for example at least about any of 60%, 70%, 80%, 90%, or more biological activity of the full length wild-type interferon), including any biosimilar, biogeneric, follow-on biologic, or follow-on protein version of an interferon taught in the art. The interferon can be from essentially any mammalian species. In various embodiments, the interferon is from a species selected from the group consisting of human, equine, bovine, rodent, porcine, lagomorph, feline, canine, murine, caprine, ovine, a non-human primate, and the like. Various such interferons have been extensively described in the literature and are well known to one of ordinary skill in the art (see, e.g., Pestka, Immunological Reviews, 202(1):8-32, 2004). FDA-approved interferons include, e.g., ROFERON®-A (Roche), INTRON® A (Schering), INFERGEN® (InterMune, Inc), AVONEX® (Biogen, Inc.), BETASERON® (Chiron Corporation) and REBIF® (EMD Serono and Pfizer).

In various embodiments, the TAA antibody-IFN fusion molecules comprise an interferon or a modified interferon that possesses, e.g., at least 10%, at least 20%, at least 30%, at least 40%, at least 50%, at least 55%, at least 60%, at least 65%, at least 70%, at least 75%, at least 80%, at least 85%, at least 90%, at least 95%, at least 96%, at least 97%, at least 98%, at least 99%, at least 100%, of the endogenous activity of the wild-type interferon having the same amino acid sequence but not attached to an antibody.

In various embodiments, the TAA antibody-IFN fusion molecules will comprise an interferon or a modified interferon that possesses, e.g., less than 10%, less than 20%, less than 30%, less than 40%, less than 50%, less than 55%, less than 60%, less than 65%, less than 70%, less than 75%, less than 80%, less than 85%, less than 90%, less than 95%, less than 96%, less than 97%, less than 98%, less than 99%, less than 100%, of the endogenous activity of the wild-type interferon having the same amino acid sequence but not attached to an antibody.

In various embodiments, the TAA antibody-IFN fusion molecules will comprise an interferon or a modified interferon that possesses, e.g., more than 5 times, more than 10 times, more than 15 times, more than 20 times, more than 25 times, more than 30 times, more than 35 times, more than 40 times, more than 50 times, more than 60 times, more than 70 times, more than 80 times, more than 90 times, more than 100 times, more than 125 times, more than 150 times, more than 175 times, more than 200 times, more than 250 times, more than 300 times, more than 400 times, more than 500 times, more than 750 times, and more than 1000 times, the endogenous activity of the wild-type interferon having the same amino acid sequence but not attached to an antibody.

Interferon activity can be assessed, for example, using the various anti-viral and anti-proliferative assays described in art (see, e.g., U.S. Pat. No. 8,563,692, U.S. Pat. Public. No. 20130230517, U.S. Pat. Public. No. 20110158905, PCT WO/2014/028502, and PCT WO/2013/059885) as well as the assays described in the Examples section below.

In various embodiments, the TAA antibody-IFN fusion molecules will show at least 10, at least 100, at least 1000, at least 10,000, or at least 100,000 fold selectivity toward cells that express the TAA to which the antibody binds over cells that do not express the TAA, when compared to interferon having the same amino acid sequence not attached to an antibody.

In various embodiments of the present invention, the interferon is an interferon mutant which comprises one or more amino acid substitutions, insertions, and/or deletions. Means of identifying such mutant interferon molecules are routine to those of skill in the art. In one illustrative approach, a library of truncated and/or mutated IFN-α is produced and screened for IFN-α activity. Methods of producing libraries of polypeptide variants are well known to those of skill in the art. Thus, for example, error-prone PCR can be used to create a library of mutant and/or truncated IFN-α (see, e.g., U.S. Pat. No. 6,365,408). The resultant library members can then be screened according to standard methods know to those of skill in the art. Thus, for example, IFN-α activity can be assayed by measuring antiviral activity against a particular test virus. Kits for assaying for IFN-α activity are commercially available (see, e.g., ILITE™ alphabeta kit by Neutekbio, Ireland).

In various embodiments of the present disclosure, the interferon mutant comprises one or more amino acid substitutions, insertions, and/or deletions. Means of identifying such modified interferon molecules are routine to those of skill in the art. In one illustrative approach, a library of truncated and/or mutated IFN-α is produced and screened for IFN-α activity. Methods of producing libraries of polypeptide variants are well known to those of skill in the art. Thus, for example, error-prone PCR can be used to create a library of mutant and/or truncated IFN-α (see, e.g., U.S. Pat. No. 6,365,408). The resultant library members can then be screened according to standard methods know to those of skill in the art. Thus, for example, IFN-α activity can be assayed by measuring antiviral activity against a particular test virus. Kits for assaying for IFN-α activity are commercially available (see, e.g., ILITE™ alphabeta kit by Neutekbio, Ireland).

The use of chemically modified interferons is also contemplated. For example, in certain embodiments, the interferon is chemically modified to increase serum half-life. Thus, for example, (2-sulfo-9-fluorenylmethoxycarbonyl)₇-interferon-α2 undergoes time-dependent spontaneous hydrolysis, generating active interferon (Shechter et al., Proc. Natl. Acad. Sci., USA, 98(3): 1212-1217, 2001). Other modifications, include for example, N-terminal modifications in including, but not limited to the addition of PEG, protecting groups, and the like (see, e.g., U.S. Pat. No. 5,824,784).

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype IFN-α2b sequence provided below as SEQ ID NO: 1 (hereinafter referred to as “IFN-α2b”):

(SEQ ID NO: 1)
CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMRS
FSLSTNLQESLRSKE

In various embodiments use of a mutated IFN-α is contemplated. Single point mutations contemplated for use herein include, but are not limited to, a series of mostly single point mutants (see Table 1 below) that are considered important to the binding affinity of IFN-α to IFN-αR1 based on published information on NMR structure with the assumption that a single point mutation may change the binding affinity but will not completely knock off the activity of IFN-α, therefore still retaining the anti-proliferative properties albeit at much higher concentrations. This will potentially improve the therapeutic index of the fusion molecules comprising an antibody fused to the interferon-alpha mutants. As described herein and as depicted in Table 1, a single mutation will be identified by the particular amino acid substitution at a specific amino acid position within the sequence of wildtype IFN-α2b provided as SEQ ID NO: 1. For example, a mutation comprising a tyrosine substituted for the full length wild type histidine at amino acid 57 is identified as H57Y.

TABLE 1
List of proposed IFN-α2b Mutant Molecules.

| | IFN-α sequence | |
|---|---|---|
| | mutations | Selection Criteria |
| M1 | H57Y, E58N, | Phage display optimization of selected IFN-α residues to increase IFN-α-IFN- |
| | Q61S | αR1 binding affinity of Site 1 |
| M2 | H57S, E58S, | Decrease the IFN-α-IFN-αR1 binding affinity at Site 1 based on triple mutations |
| | Q61S | predicted to result in a loss of binding contacts between IFN-α and IFN-αR1 |
| M3 | H57A | Decrease the IFN-α-IFN-αR1 binding affinity at Site 1 similar to M2 but only |
| | | single point |
| M4 | E58A | Decrease the IFN-α-IFN-αR1 binding affinity at Site 1 similar to M2 but only |
| | | single point |
| M5 | Q61A | Decrease the IFN-α-IFN-αR1 binding affinity at Site 1 similar to M2 but only |
| | | single point |
| M6 | R149A | Decrease the IFN-α-IFN-αR1 binding affinity at Site 2 based on loss of binding |
| | | contacts |
| M7 | R162A | Decrease the IFN-α-IFN-αR1 binding affinity at Site 2 based on loss of binding |
| | | contacts |
| M8 | R149A, R162A | Decrease the IFN-α-IFN-αR1 binding affinity at Site 2 based on loss of binding |
| | | contacts |
| M9 | L30A | Decrease the IFN-α-IFN-αR1 binding affinity at Site 2 based on loss of binding |
| | | contacts |
| M10 | D35E | Alter the IFN-α-IFN-αR1 binding at Site 2 based on minimal change in structure |
| M11 | E165D | Alter the IFN-α-IFN-αR1 binding at Site 2 based on minimal change in structure |
| M12 | L26A | Alter the IFN-α-IFN-αR1 binding at Site 2 based on minimal change in structure |
| M13 | F27A | Alter the IFN-α-IFN-αR1 binding at Site 2 based on minimal change in structure |
| M14 | L153A | Alter the IFN-α-IFN-αR1 binding at Site 2 based on minimal change in structure |
| M15 | A145V | Alter the IFN-α-IFN-αR1 binding at Site 2 based on minimal change in structure |

In some embodiments, the mutant interferon has at least 70%, at least 75%, at least 80%, at least 85%, at least 90%, at least 95%, at least 96%, at least 97%, at least 98%, at least 99%, at least 1×, at least 1.5×, at least 2×, at least 2.5×, or at least 3× activity of wildtype IFN-α2b provided below as SEQ ID NO: 1. In some embodiments, the mutant interferon has less than any of about 70%, 75%, 80%, 85%, 90%, or 95%, activity of wildtype IFN-α2b provided below as SEQ ID NO: 1. In various embodiments, the interferon is an IFN-α2b mutant molecule wherein the arginine at amino acid residue 149 of SEQ ID NO: 1 is replaced with an alanine (R149A) and the arginine at amino acid residue 162 of SEQ ID NO: 1 is replaced with an alanine (R162A). This IFN-α2b mutant molecule is referred to hereinafter as “IFN-α2b-M8”. The amino acid sequence of IFN-α2b-M8 is provided below as SEQ ID NO: 2.

(SEQ ID NO: 2)
CDLPQTHSLGSRRTLMLLAQMRRISLFSCLKDRHDFGFPQEEFGNQFQKA
ETIPVLHEMIQQIFNLFSTKDSSAAWDETLLDKFYTELYQQLNDLEACVI
QGVGVTETPLMKEDSILAVRKYFQRITLYLKEKKYSPCAWEVVRAEIMAS
FSLSTNLQESLASKE

Additional interferon mutants contemplated for use include those described in, e.g., PCT WO 2013/059885 (Wilson et al.), and U.S. Pat. No. 8,258,263 (Morrison et al), each of which is hereby incorporated by reference in its entirety for the interferon mutants and sequences provided therein. In various embodiments, the interferon is an IFN-α2b mutant molecule having the amino acid sequence set forth in SEQ ID NO: 1, and comprising one or more single point mutations selected from L15A, A19W, R22A, R23A, S25A, L26A, F27A, L30A, L30V, K31A, D32A, R33K, R33A, R33Q, H34A, D35E, Q40A, H57A, H57S, H57Y, E58A, E58N, E58S, Q61A, Q61S, D114R, L117A, R120A, R125A, R125E, K131A, E132A, K133A, K134A, R144A, R144D, R144E, R144G, R144H, R144I, R144K, R144L, R144N, R144Q, R144S, R144T, R144V, R144Y, A145D, A145E, A145G, A145H, A145I, A145K, A145L, A145M, A145N, A145Q, A145R, A145S, A145T, A145V, A145Y, M148A, R149A, S152A, L153A, N156A, R162A, or E165D.

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype IFN-α14 sequence provided below as SEQ ID NO: 3 (referred to hereinafter as “IFN-α14”). In some embodiments, the interferon has at least 70%, at least 75%, at least 80%, at least 85%, at least 90%, at least 95%, at least 96%, at least 97%, at least 98%, at least 99%, at least 1×, at least 1.5×, at least 2×, at least 2.5×, or at least 3× activity of IFN-α14 provided below as SEQ ID NO: 3. In some embodiments, the interferon has less than any of about 70%, 75%, 80%, 85%, 90%, or 95%, activity of IFN-α14 provided below as SEQ ID NO: 3:

(SEQ ID NO: 3)
CNLSQTHSLNNRRTLMLMAQMRRISPFSCLKDRHDFEFPQEEFDGNQF
QKAQAISVLHEMMQQTFNLFSTKNSSAAWDETLLEKFYIELFQQMNDL
EACVIQEVGVEETPLMNEDSILAVKKYFQRITLYLMEKKYSPCAWEVV
RAEIMRSLSFSTNLQKRLRRKD

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype IFN-α sequence selected from the group consisting of IFN-α5 (NP_002160.1), IFN-α6 (NP_066282.1), IFN-α7 (NP_066401.1), IFN-α8 (NP_002161.2), IFN-α10 (NP_002162.1), IFN-α16 (NP_002164.1), IFN-α17 (NP_067091.1), and IFN-α21 (NP_002166.2).

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype IFN-β-1a sequence provided below as SEQ ID NO: 4. In some embodiments, the mutant interferon has at least 70%, at least 75%, at least 80%, at least 85%, at least 90%, at least 95%, at least 96%, at least 97%, at least 98%, at least 99%, at least 1×, at least 1.5×, at least 2×, at least 2.5×, or at least 3× activity of wildtype IFN-β-1a provided below as SEQ ID NO: 4. In some embodiments, the mutant interferon has less than any of about 70%, 75%, 80%, 85%, 90%, or 95%, activity of wildtype IFN-β-1a provided below as SEQ ID NO: 4:

(SEQ ID NO: 4)
KEDFTRGKLMSSLHLKRYYGRILHYLKAKEYSHCAWTIVRVEILRNFYFI
NRLTGYLRN

In various embodiments, the interferon contains an amino acid sequence that shares an observed homology of, e.g., at least 70%, at least 75%, at least 80%, at least 85%, at least 90%, at least 95%, at least 96%, at least 97%, at least 98%, or at least 99% with the wildtype IFN-β-1 b sequence provided below as SEQ ID NO: 5. In some embodiments, the mutant interferon has at least 70%, at least 75%, at least 80%, at least 85%, at least 90%, at least 95%, at least 96%, at least 97%, at least 98%, at least 99%, at least 1×, at least 1.5×, at least 2×, at least 2.5×, or at least 3× activity of wildtype IFN-β-1b provided below as SEQ ID NO: 5. In some embodiments, the mutant interferon has less than any of about 70%, 75%, 80%, 85%, 90%, or 95%, activity of wildtype IFN-β-1b provided below as SEQ ID NO: 5:

(SEQ ID NO: 5)
MSYNLLGFLQRSSNFQSQKLLWQLNGRLEYCLKDRMNFDIPEEIKQLQQF
QKEDAALTIYEMLQNIFAIFRQDSSSTGWNETIVENLLANVYHQINHLKT
VLEEKLEKEDFTRGKLMSSLHLKRYYGRILHYLKAKEYSHCAWTIVRVEI
LRNFYFINRLTGYLRN

In various embodiments use of a mutated IFN-β is contemplated. A mutated IFN-β comprising a serine substituted for the naturally occurring cysteine at amino acid 17 of IFN-β-1a has also been demonstrated to show efficacy (Hawkins et al., Cancer Res., 45:5914-5920, 1985). Certain C-terminally truncated IFN-β-1a's have been shown to have increased activity (see, e.g., U.S. Patent Publication 2009/0025106 A1). Accordingly, in certain embodiments the interferons used in the fusion molecules described herein include the C-terminally truncated IFN-β described as IFN-Δ1, IFN-Δ2, IFN-Δ3, IFN-Δ4, IFN-Δ5, IFN-Δ6, IFN-Δ7, IFN-Δ8, IFN-Δ9, IFN-Δ10 in US 2009/0025106 A1. This reference is incorporated by reference in its entirety herein for purposes of the interferon mutants and sequences provided therein.

### Tumor Associated Antigen Antibodies

The methods of the present invention utilize isolated non-occurring genetically engineered TAA Ab-IFN fusion molecules comprising at least one tumor associated antigen antibody, or antigen-binding fragment thereof, attached to at least one interferon, or interferon mutant molecule.

A wide variety of tumor associated antigens and tumor markers have been described in the literature and are well known to one of ordinary skill in the art. The TAA Ab-IFN fusion molecules used in the methods of the present invention may comprise an antibody, or antigen binding antibody fragment, specific to any of the tumor associated antigens described in the art, including any biosimilar, biogeneric, follow-on biologic, or follow-on protein version of any TAA described in the art. The TAA can be any peptide, polypeptide, protein, nucleic acid, lipid, carbohydrate, or small organic molecule, or any combination thereof, against which the skilled artisan wishes to induce an immune response.

In various embodiments, the TAA contemplated for use includes, but is not limited to those provided in Table 2. Each associated reference is incorporated herein by reference for the purpose of identifying the referenced tumor markers.

TABLE 2
Illustrative Tumor Markers

| Marker | Reference |
|---|---|
| 5 alpha reductase | Delos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (1998) J Exp Med., 188(6): 1185-1190 |
| BAGE | Boel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al. (1996) Blood, 87(11): 4770-4779 |
| CA-125 (Mucin 16) | Bast et al. (1998) Int J Biol Markers, 13(4): 179-187 |
| CASP-8/FLICE | Mandruzzato et al. (1997)J Exp Med., 186(5): 785-793. |
| Cathepsins | Thomssen et al. (1995)Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 | Jarvis et al. (1997) Int J Cancer, 71(6): 1049-1055 |
| CDC27 | Wang et al. (1999) Science, 284(5418): 1351-1354 |
| CDK4 | Wolfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al. (1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunther. 44(4): 239-247 |
| gp-100-in4 | Kirkin et al. (1998) APMIS, 106(7): 665-679 |
| gp15 | Maeurer et al. (1996) Melanoma Res., 6(1): 11-24 |
| gp75/TRP-1 | Lewis et al. (1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al. (1991)Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) BiochemBiophysRes Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Gueguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 (CO17-1A) |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996)Proc. Natl. Acad. Sci., USA, 93(1): 136-140 |
| MIC A/B | Groh et al. (1998) Science, 279: 1737-1740 |
| MT-MMP's, such as | Sato and Seiki (1996) J Biochem (Tokyo), 119(2): 209-215 |
| MMP2, MMP3, MMP7, | |
| MMP9 | |
| Mox1 | Candia et al. (1992) Development, 116(4): 1123-1136 |
| Mucin, such as MUC-1, | Lewis and Houghton (1995) Semin Cancer Biol., 6(6): 321-327 |
| MUC-2, MUC-3, MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grondahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) BiochemBiophysResComm., 130(1): 293-300 |
| Progenipoietin—PSA | Sanda et al. (1999) Urology, 53(2): 260-266. |
| PSM | Kawakami et al. (1997) Cancer Res., 57(12): 2321-2324 |
| RAGE-1 | Gaugler et al. (1996) Immunogenetics, 44(5): 323-330 |
| Rb | Dosaka-Akita et al. (1997) Cancer, 79(7): 1329-1337 |
| RCAS1 | Sonoda et al. (1996) Cancer, 77(8): 1501-1509. |
| SART-1 | Kikuchi et al. (1999) Int J Cancer, 81(3): 459-466 |
| SSX gene | Gure et al. (1997) Int J Cancer, 72(6): 965-971 family |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990) Cancer Res., 50(16): 4872-4879 |
| TGF-α | Imanishi et al. (1989) Br J Cancer, 59(5): 761-765 |
| TGF-β | Picon et al. (1998) CancerEpidemiolBiomarkerPrey, 7(6): 497-504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al. (1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |

In various embodiments, the TAA contemplated for use includes, but is not limited to those provided in Table 3.

TABLE 3

| Tumor Associated Antigen | RefSeq (protein) |
|---|---|
| Her2/neu | NP_001005862 |
| Her3 | NP_001005915 |
| Her4 | NP_001036064 |
| EGF | NP_001171601 |
| EGFR | NP_005219 |
| CD2 | NP_001758 |
| CD3 | NM_000732 |
| CD5 | NP_055022 |
| CD7 | NP_006128 |
| CD13 | NP_001141 |
| CD19 | NP_001171569 |
| CD20 | NP_068769 |
| CD21 | NP_001006659 |
| CD23 | NP_001193948 |
| CD30 | NP_001234 |
| CD33 | NP_001234.3 |
| CD34 | NP_001020280 |
| CD38 | NP_001766 |
| CD46 | NP_002380 |
| CD55 | NP_000565 |
| CD59 | NP_000602 |
| CD69 | NP_001772 |
| CD70 | NM_001252 |
| CD71 | NP_001121620 |
| CD97 | NP_001020331 |
| CD117 | NP_000213 |
| CD127 | NP_002176 |
| CD134 | NP_003318 |
| CD137 | NP_001552 |
| CD138 | NP_001006947 |
| CD146 | NP_006491 |
| CD147 | NP_001719 |
| CD152 | NP_001032720 |
| CD154 | NP_000065 |
| CD195 | NP_000570 |
| CD200 | NP_001004196 |
| CD212 | NP_001276952 |
| CD223 | NP_002277 |
| CD253 | NP_001177871 |
| CD272 | NP_001078826 |
| CD274 | NP_001254635 |
| CD276 | NP_001019907 |
| CD278 | NP_036224 |
| CD279 | NP_005009 |
| CD309 (VEGFR2) | NP_002244 |
| DR6 | NP_055267 |
| PD-L1 | NP_001254635 |
| Kv1.3 | NP_002223 |
| 5E10 | NP_006279 |
| MUC1 | NP_001018016 |
| uPA | NM_002658 |
| SLAMF7 (CD319) | NP_001269517 |
| MAGE 3 | NP_005353 |
| MUC 16 (CA-125) | NP_078966 |
| KLK3 | NP_001025218 |
| K-ras | NP_004976 |
| Mesothelin | NP_001170826 |
| p53 | NP_000537 |
| Survivin | NP_001012270 |
| G250 (Renal Cell Carcinoma | GenBank CAB82444.1 |
| Antigen) | |
| PSMA | NP_001014986 |
| Endoplasmin (GRP94) | NM_003299 |

Methods of generating antibodies that bind to the TAAs described herein are known to those skilled in the art. For example, a method for generating a monoclonal antibody that binds specifically to a targeted antigen polypeptide may comprise administering to a mouse an amount of an immunogenic composition comprising the targeted antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the targeted antigen polypeptide. Once obtained, a hybridoma can be propagated in a cell culture, optionally in culture conditions where the hybridoma-derived cells produce the monoclonal antibody that binds specifically to targeted antigen polypeptide. The monoclonal antibody may be purified from the cell culture. A variety of different techniques are then available for testing an antigen/antibody interaction to identify particularly desirable antibodies.

Other suitable methods of producing or isolating antibodies of the requisite specificity can used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. (U.S.A.), 90: 2551-2555, 1993; Jakobovits et al., Nature, 362: 255-258, 1993; Lonberg et al., U.S. Pat. No. 5,545,806; and Surani et al., U.S. Pat. No. 5,545,807.

Antibodies can be engineered in numerous ways. They can be made as single-chain antibodies (including small modular immunopharmaceuticals or SMIPs™), Fab and F(ab′)₂ fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science, 240:1041-1043, 1988; Liu et al., Proc. Natl. Acad. Sci. (U.S.A.), 84:3439-3443, 1987; Liu et al., J. Immunol., 139:3521-3526, 1987; Sun et al., Proc. Natl. Acad. Sci. (U.S.A.), 84:214-218, 1987; Nishimura et al., Canc. Res., 47:999-1005, 1987; Wood et al., Nature, 314:446-449, 1985; and Shaw et al., J. Natl Cancer Inst., 80:1553-1559, 1988).

Methods for humanizing antibodies have been described in the art. In some embodiments, a humanized antibody has one or more amino acid residues introduced from a source that is nonhuman, in addition to the nonhuman CDRs. Humanization can be essentially performed following the method of Winter and co-workers (Jones et al., Nature, 321:522-525, 1986; Riechmann et al., Nature, 332:323-327, 1988; Verhoeyen et al., Science, 239:1534-1536, 1988), by substituting hypervariable region sequences for the corresponding sequences of a human antibody. Accordingly, such “humanized” antibodies are chimeric antibodies (U.S. Pat. No. 4,816,567) wherein substantially less than an intact human variable region has been substituted by the corresponding sequence from a nonhuman species. In practice, humanized antibodies are typically human antibodies in which some hypervariable region residues and possibly some framework region residues are substituted by residues from analogous sites in rodent antibodies.

U.S. Pat. No. 5,693,761 to Queen et al, discloses a refinement on Winter et al. for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable region sequences are compared to a particular mouse sequence and the percentage identity between correspondent framework residues is calculated. The human variable region with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6 Å of any CDR residue.

In other approaches, the importance of particular framework amino acid residues is determined experimentally once a low-avidity humanized construct is obtained, by reversion of single residues to the mouse sequence and assaying antigen binding as described by Riechmann et al, 1988. Another example approach for identifying important amino acids in framework sequences is disclosed by U.S. Pat. No. 5,821,337 to Carter et al, and by U.S. Pat. No. 5,859,205 to Adair et al. These references disclose specific Kabat residue positions in the framework, which, in a humanized antibody may require substitution with the correspondent mouse amino acid to preserve avidity.

Another method of humanizing antibodies, referred to as “framework shuffling”, relies on generating a combinatorial library with nonhuman CDR variable regions fused in frame into a pool of individual human germline frameworks (Dall'Acqua et al., Methods, 36:43, 2005). The libraries are then screened to identify clones that encode humanized antibodies which retain good binding.

The choice of human variable regions, both light and heavy, to be used in making the humanized antibodies is very important to reduce antigenicity. According to the so-called “best-fit” method, the sequence of the variable region of a rodent antibody is screened against the entire library of known human variable-domain sequences. The human sequence that is closest to that of the rodent is then accepted as the human framework region (framework region) for the humanized antibody (Sims et al., J. Immunol., 151:2296, 1993; Chothia et al., J. Mol. Biol., 196:901, 1987). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. (U.S.A.), 89:4285, 1992; Presta et al., J. Immunol., 151:2623, 1993).

The choice of nonhuman residues to substitute into the human variable region can be influenced by a variety of factors. These factors include, for example, the rarity of the amino acid in a particular position, the probability of interaction with either the CDRs or the antigen, and the probability of participating in the interface between the light and heavy chain variable domain interface. (See, for example, U.S. Pat. Nos. 5,693,761, 6,632,927, and 6,639,055). One method to analyze these factors is through the use of three-dimensional models of the non-human and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, nonhuman residues can be selected and substituted for human variable region residues in order to achieve the desired antibody characteristic, such as increased affinity for the target antigen(s).

Methods for making fully human antibodies have been described in the art. By way of example, a method for producing a TAA antibody or antigen-binding fragment thereof comprises the steps of synthesizing a library of human antibodies on phage, screening the library with TAA or an antibody-binding portion thereof, isolating phage that bind TAA, and obtaining the antibody from the phage. By way of another example, one method for preparing the library of antibodies for use in phage display techniques comprises the steps of immunizing a non-human animal comprising human immunoglobulin loci with TAA or an antigenic portion thereof to create an immune response, extracting antibody-producing cells from the immunized animal; isolating RNA encoding heavy and light chains of antibodies of the disclosure from the extracted cells, reverse transcribing the RNA to produce cDNA, amplifying the cDNA using primers, and inserting the cDNA into a phage display vector such that antibodies are expressed on the phage. Recombinant anti-TAA antibodies of the disclosure may be obtained in this way.

Again, by way of example, recombinant human anti-TAA antibodies of the disclosure can also be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology, 9:1370-1372 (1991); Hay et al., Hum. Antibod. Hybridomas, 3:81-85, 1992; Huse et al., Science, 246:1275-1281, 1989; McCafferty et al., Nature, 348:552-554, 1990; Griffiths et al., EMBO J., 12:725-734, 1993; Hawkins et al., J. Mol. Biol., 226:889-896, 1992; Clackson et al., Nature, 352:624-628, 1991; Gram et al., Proc. Natl. Acad. Sci. (U.S.A.), 89:3576-3580, 1992; Garrad et al., Bio/Technology, 9:1373-1377, 1991; Hoogenboom et al., Nuc. Acid Res., 19:4133-4137, 1991; and Barbas et al., Proc. Natl. Acad. Sci. (U.S.A.), 88:7978-7982, 1991), all incorporated herein by reference.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.—Fremont, Calif.). XenoMouse™ mice are engineered mouse strains that comprise large fragments of human immunoglobulin heavy chain and light chain loci and are deficient in mouse antibody production. See, e.g., Green et al., Nature Genetics, 7:13-21, 1994 and U.S. Pat. Nos. 5,916,771, 5,939,598, 5,985,615, 5,998,209, 6,075,181, 6,091,001, 6,114,598, 6,130,364, 6,162,963 and 6,150,584. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics, 15:146-156, 1997; Green and Jakobovits, J. Exp. Med., 188:483-495, 1998; and WO 98/24893.

In various embodiments, the fusion molecules of the present disclosure utilize an antibody or antigen-binding fragment thereof that is selected from the group consisting of a polyclonal antibody, a monoclonal antibody, a recombinant antibody, a diabody, a chimerized or chimeric antibody or antigen-binding fragment thereof, a humanized antibody or antigen-binding fragment thereof, a fully human antibody or antigen-binding fragment thereof, a CDR-grafted antibody or antigen-binding fragment thereof, a single chain antibody, an Fv, an Fd, an Fab, an Fab′, or an F(ab′)₂, and synthetic or semi-synthetic antibodies.

In various embodiments, the fusion molecules of the present disclosure utilize an antibody or antigen-binding fragment that binds to a TAA with a dissociation constant (K_{D}) of, e.g., at least about 1×10⁻³ M, at least about 1×10⁻⁴ M, at least about 1×10⁻⁵ M, at least about 1×10⁻⁶ M, at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M. In various embodiments, the fusion molecules of the present disclosure utilize an antibody or antigen-binding fragment that binds to a TAA with a dissociation constant (K_{D}) in the range of, e.g., at least about 1×10⁻³ M to at least about 1×10⁻⁴ M, at least about 1×10⁻⁴ M to at least about 1×10⁻⁵ M, at least about 1×10⁻⁵ M to at least about 1×10⁻⁶ M, at least about 1×10⁻⁶ M to at least about 1×10⁻⁷ M, at least about 1×10⁻⁷ M to at least about 1×10⁻⁸ M, at least about 1×10⁻⁸ M to at least about 1×10⁻⁹ M, at least about 1×10⁻⁹ M to at least about 1×10⁻¹⁰ M, at least about 1×10⁻¹⁰ M to at least about 1×10⁻¹¹ M, or at least about 1×10⁻¹¹ M to at least about 1×10⁻¹² M.

In various embodiments, the fusion molecules of the present disclosure utilize an antibody or antigen-binding fragment that cross-competes for binding to the same epitope on the TAA as a reference antibody which comprises the heavy chain variable region and light chain variable region set forth in the references and sequence listings provided herein.

Anti-HER2 Antibodies. The ergB 2 gene, more commonly known as (HER2/neu), is an oncogene encoding a transmembrane receptor. Several antibodies have been developed against HER2/neu, including trastuzumab (e.g., HERCEPTIN®); Fornier et al., Oncology (Huntingt) 13: 647-58 (1999)), TAB-250 (Rosenblum et al., Clin. Cancer Res. 5: 865-74 (1999)), BACH-250 (Id.), TA1 (Maier et al., Cancer Res. 51: 5361-9 (1991)), and the mAbs described in U.S. Pat. Nos. 5,772,997; 5,770,195 (mAb 4D5; ATCC CRL 10463); and U.S. Pat. No. 5,677,171, each of which is hereby incorporated by reference in its entirety for purposes of providing such antibodies and antigen-binding fragments. In various embodiments the antibody is an anti-HER2/neu antibody which comprises a heavy chain having an amino acid sequence as set forth in SEQ ID NO: 6:

(SEQ ID NO: 6)
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK

In various embodiments, the heavy chain of the anti-HER2/neu antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 6. In various embodiments, the heavy chain of the anti-HER2/neu antibody comprises the CDR1, CDR2, and CDR3 sequences of SEQ ID NO: 6.

In various embodiments the antibody is an anti-HER2/neu antibody which comprises a light chain having an amino acid sequence as set forth in SEQ ID NO: 7:

(SEQ ID NO: 7)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFEPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In various embodiments, the light chain of the anti-HER2/neu antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 7. In various embodiments, the light chain of the anti-HER2/neu antibody comprises the CDR1, CDR2, and CDR3 sequences of SEQ ID NO: 7.

In various embodiments, the anti-HER2/neu antibody specifically binds to the same epitope as the antibody having a heavy chain having the amino acid sequence of SEQ ID NO: 6 and a light chain having the amino acid sequence of SEQ ID NO: 7. In various embodiments, the anti-HER2/neu antibody competes for binding to the HER2/neu antigen with the antibody having a heavy chain having the amino acid sequence of SEQ ID NO: 6 and a light chain having the amino acid sequence of SEQ ID NO: 7.

Anti-CD20 Antibodies. The FDA approved anti-CD20 antibody, Rituximab (IDEC C2B8; RITUXAN; ATCC No. HB 11388) has also been used to treat humans. Ibritumomab, is the murine counterpart to Rituximab (Wiseman et al., Clin. Cancer Res. 5: 3281s-6s (1999)). Other reported anti-CD20 antibodies include the anti-human CD20 mAb 1F5 (Shan et al., J. Immunol 162: 6589-95 (1999)), the single chain Fv anti-CD20 mouse mAb 1H4 (Haisma et al., Blood 92: 184-90 (1998)) and anti-B1 antibody (Liu et al., J. Clin. Oncol. 16: 328-70 (1998)) each of which is hereby incorporated by reference in its entirety for purposes of providing such antibodies and antigen-binding fragments. In various embodiments the antibody is an chimeric anti-CD20 antibody which comprises a heavy chain having an amino acid sequence as set forth in SEQ ID NO: 8:

(SEQ ID NO: 8)
QVQLQQPGAELVKPGASVKMSCKASGYTFTSYNMHWVKQTPGRGLEWIGA
IYPGNGDTSYNQKFKGKATLTADKSSSTAYMQLSSLTSEDSAVYYCARST
YYGGDWYFNVWGAGTTVTVSAASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKIDTLMISRTPEVICVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK

In various embodiments, the heavy chain of the anti-CD20 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 8. In various embodiments, the heavy chain of the anti-CD20 antibody comprises the CDR1, CDR2, and CDR3 sequences of SEQ ID NO: 8.

In various embodiments the antibody is an anti-CD20 antibody which comprises a light chain having an amino acid sequence as set forth in SEQ ID NO: 9:

(SEQ ID NO: 9)
QIVLSQSPAILSASPGEKVTMTCRASSSVSYIHWFQQKPGSSPKPWIYAT
SNLASGVPVRFSGSGSGTSYSLTISRVEAEDAATYYCQQWTSNPPTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In various embodiments, the light chain of the anti-CD20 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 9. In various embodiments, the light chain of the anti-CD20 antibody comprises the CDR1, CDR2, and CDR3 sequences of SEQ ID NO: 9.

In various embodiments, the anti-CD20 antibody specifically binds to the same epitope as the antibody having a heavy chain having the amino acid sequence of SEQ ID NO: 8 and a light chain having the amino acid sequence of SEQ ID NO: 9. In various embodiments, the anti-CD20 antibody competes for binding to the CD20 antigen with the antibody having a heavy chain having the amino acid sequence of SEQ ID NO: 8 and a light chain having the amino acid sequence of SEQ ID NO: 9.

Anti-CD138 Antibodies. Murine and chimeric anti-CD138 antibodies are described in, e.g., US Patent Application Publication No. 20070183971 (Goldmakher) and 20090232810 (Kraus et al) each of which is hereby incorporated by reference in its entirety for purposes of providing such antibodies and antigen-binding fragments. In various embodiments the antibody is an anti-CD138 antibody which comprises a heavy chain having an amino acid sequence as set forth in SEQ ID NO: 10:

(SEQ ID NO: 10)
QVQLQQSGSELMMPGASVKISCKATGYTFSNYWIEWVKQRPGHGLEWIGE
ILPGTGRTlYNEKFKGKATFTADISSNTVQMQLSSLTSEDSAVYYCARRD
YYGNFYYAMDYWGQGTSVIVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSP
GK

In various embodiments, the heavy chain of the anti-CD138 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 10. In various embodiments, the heavy chain of the anti-CD138 antibody comprises the CDR1, CDR2, and CDR3 sequences of SEQ ID NO: 10.

In various embodiments the antibody is an anti-CD138 antibody which comprises a light chain having an amino acid sequence as set forth in SEQ ID NO: 11:

(SEQ ID NO: 11)
DIQMTQSTSSLSASLGDRVTISCSASQGINNYLNWYQQKPDGTVELLIYY
TSTLQSGVPSRFSGSGSGTDYSLTISNLEPEDIGTYYCQQYSKLPRTFGG
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In various embodiments, the light chain of the anti-CD138 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 11. In various embodiments, the light chain of the anti-CD138 antibody comprises the CDR1, CDR2, and CDR3 sequences of SEQ ID NO: 11.

In various embodiments, the anti-CD138 antibody specifically binds to the same epitope as an antibody having a heavy chain having the amino acid sequence of SEQ ID NO: 10 and a light chain having the amino acid sequence of SEQ ID NO: 11. In various embodiments, the anti-CD138 antibody competes for binding to the CD138 antigen with an antibody having a heavy chain having the amino acid sequence of SEQ ID NO: 10 and a light chain having the amino acid sequence of SEQ ID NO: 11.

Anti-GRP94 (endoplasmin) Antibodies. Isolated monoclonal antibodies, including fully human antibodies that specifically bind endoplasmin (GRP94) and use in detecting tumors that express endoplasmin, methods of treatment using the antibodies, and immunoconjugates comprising the antibodies are described in U.S. Pat. No. 8,497,354 (Ferrone et al.) and US 20040001789 (Young et al), each of which is hereby incorporated by reference in its entirety for purposes of providing such antibodies and antigen-binding fragments. In various embodiments the antibody is a GRP94 antibody which comprises a heavy chain having an amino acid sequence as set forth in SEQ ID NO: 12:

(SEQ ID NO: 12)
QVQLVQSGAEVKKPGASVKVSCKASGYTFTSYAMHWVRQAPGQRLEWMGW
INAGNGNTKYSQKFQGRVTITRDTSASTAYMELSSLRSEDTAVYYCARAH
FDYWGQGTLVTVSAASTKGPSVFPLAPSSKSTSGGTAALGCLVKDYFPEP
VTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK

In various embodiments, the heavy chain of the GRP94 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 12. In various embodiments, the heavy chain of the anti-GRP94 antibody comprises the CDR1, CDR2, and CDR3 sequences of SEQ ID NO: 12.

In various embodiments the antibody is a GRP94 antibody which comprises a light chain having an amino acid sequence as set forth in SEQ ID NO: 13:

(SEQ ID NO: 13)
EIELTQSPSSLSASVGDRVTITCRASQSISSYLNWYQQKPGKAPKLLIYA
ASSLQSGVPSRFSGSGSGTDFTLTISSLQPEDFATYYCQQSYSTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

In various embodiments, the light chain of the GRP94 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 13. In various embodiments, the light chain of the anti-GRP94 antibody comprises the CDR1, CDR2, and CDR3 sequences of SEQ ID NO: 13.

In various embodiments, the anti-GRP94 antibody specifically binds to the same epitope as an antibody having a heavy chain having the amino acid sequence of SEQ ID NO: 12 and a light chain having the amino acid sequence of SEQ ID NO: 13. In various embodiments, the anti-GRP94 antibody competes for binding to the GRP94 antigen with an antibody having a heavy chain having the amino acid sequence of SEQ ID NO: 12 and a light chain having the amino acid sequence of SEQ ID NO: 13.

Anti-CD33 Antibodies. CD33 is a glycoprotein expressed on early myeloid progenitor and myeloid leukemic (e.g., acute myelogenous leukemia, AML) cells, but not on stem cells. An IgG₁ monoclonal antibody was prepared in mice (M195) and also in a humanized form (HuM195) that reportedly has antibody-dependent cellular cytotoxicity (Kossman et al., Clin. Cancer Res. 5: 2748-55 (1999)). An anti-CD33 immunoconjugate (CMA-676) consisting of a humanized anti-CD33 antibody linked to the antitumor antibiotic calicheamicin reportedly demonstrated selective ablation of malignant hematopoiesis in some AML patients (Sievers et al., Blood 93: 3678-84 (1999) each of which is hereby incorporated by reference in its entirety for purposes of providing such antibodies and antigen-binding fragments. In various embodiments the antibody is an anti-CD33 antibody which comprises a heavy chain having an amino acid sequence as set forth in SEQ ID NO: 14:

(SEQ ID NO: 14)
QVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK

In various embodiments, the heavy chain of the anti-CD33 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 14. In various embodiments, the heavy chain of the anti-CD33 antibody comprises the CDR1, CDR2, and CDR3 sequences of SEQ ID NO: 14.

In various embodiments the antibody is an anti-CD33 antibody which comprises a light chain having an amino acid sequence as set forth in SEQ ID NO: 15:

(SEQ ID NO: 15)
DIQLTQSPSTLSASVGDRVTITCRASESLDNYGIRFLTWFQQKPGKAPKL
LMYAASNQGSGVPSRFSGSGSGTEFTLTISSLQPDDFATYYCQQTKEVPW
SFGQGTKVEVKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

In various embodiments, the light chain of the anti-CD33 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 15. In various embodiments, the light chain of the anti-CD33 antibody comprises the CDR1, CDR2, and CDR3 sequences of SEQ ID NO: 15.

In various embodiments, the anti-CD33 antibody specifically binds to the same epitope as an antibody having a heavy chain having the amino acid sequence of SEQ ID NO: 14 and a light chain having the amino acid sequence of SEQ ID NO: 15. In various embodiments, the anti-CD33 antibody competes for binding to the CD33 antigen with an antibody having a heavy chain having the amino acid sequence of SEQ ID NO: 14 and a light chain having the amino acid sequence of SEQ ID NO: 15.

Anti-CD70 (CD27L) Antibodies. Antibodies that bind CD70 are described in, e.g., U.S. Pat. No. 7,491,390 (Law et al) and U.S. Pat. No. 8,124,738 (Terret et al) each of which is hereby incorporated by reference in its entirety for purposes of providing such antibodies and antigen-binding fragments. In various embodiments the antibody is an anti-CD70 antibody which comprises a heavy chain variable region having an amino acid sequence as set forth in SEQ ID NO: 16:

(SEQ ID NO: 16)
QVQLVESGGGVVQPGRSLRLSCAASGFTFSSYIMHWVRQAPGKGLEWVAV
ISYDGRNKYYADSVKGRFTISRDNSKNTLYLQMNSLRAEDTAVYYCARDT
DGYDFDYWGQGTLVTVSS

In various embodiments, the heavy chain variable region of the anti-CD70 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 16. In various embodiments, the heavy chain of the anti-CD70 antibody comprises the CDR1, CDR2, and CDR3 sequences of SEQ ID NO: 16.

In various embodiments the antibody is an anti-CD70 antibody which comprises a light chain variable region having an amino acid sequence as set forth in SEQ ID NO: 17:

(SEQ ID NO: 17)
EIVLTQSPATLSLSPGERATLSCRASQSVSSYLAWYQQKPGQAPRLLIYD
ASNRATGIPARFSGSGSGTDFTLTISSLEPEDFAVYYCQQRTNWPLTFGG
GTKVEIK

In various embodiments, the light chain variable region of the anti-CD70 antibody has an amino acid sequence that shares an observed homology of, e.g., at least about 75%, at least about 80%, at least about 85%, at least about 90%, 91%, 92%, 93%, 94%, 95%, 96%, 97%, 98%, or at least about 99% with the sequence of SEQ ID NO: 17. In various embodiments, the light chain of the anti-CD70 antibody comprises the CDR1, CDR2, and CDR3 sequences of SEQ ID NO: 17.

In various embodiments, the anti-CD70 antibody specifically binds to the same epitope as an antibody having a heavy chain variable region having the amino acid sequence of SEQ ID NO: 16 and a light chain variable region having the amino acid sequence of SEQ ID NO: 17. In various embodiments, the anti-CD70 antibody competes for binding to the CD70 antigen with an antibody having a heavy chain having the amino acid sequence of SEQ ID NO: 16 and a light chain having the amino acid sequence of SEQ ID NO: 17.

### Fusion Molecules

Generally speaking, the TAA antibody molecule and interferon molecule of the TAA Ab-IFN fusion molecule can be joined together in any order. Thus, for example, the interferon molecule(s) can be joined to either the amino or carboxy terminal of the antibody. Alternatively, the antibody can be joined to either the amino or carboxy terminal of the interferon molecule. In various embodiments, the antibody and interferon molecule are linked directly to each other without an intervening peptide linker sequence and synthesized using recombinant DNA methodology. By “linked” we mean that the first and second sequences are associated such that the second sequence is able to be transported by the first sequence to a target cell, i.e., fusion molecules in which the antibody is linked to a IFN-α molecule via their polypeptide backbones through genetic expression of a DNA molecule encoding these proteins, directly synthesized proteins, and coupled proteins in which pre-formed sequences are associated by a cross-linking agent.

In various embodiments, the antibody portion is chemically conjugated to the interferon molecule. Means of chemically conjugating molecules are well known to those of skill. The procedure for conjugating two molecules varies according to the chemical structure of the agent. Polypeptides typically contain variety of functional groups; e.g., carboxylic acid (COOH) or free amine (—NH₂) groups, that are available for reaction with a suitable functional group on the other peptide, or on a linker to join the molecules thereto. Alternatively, the antibody and/or the interferon can be derivatized to expose or attach additional reactive functional groups. The derivatization can involve attachment of any of a number of linker molecules such as those available from Pierce Chemical Company, Rockford Ill. A bifunctional linker having one functional group reactive with a group on the antibody and another group reactive on the interferon, can be used to form the desired conjugate. Alternatively, derivatization can involve chemical treatment of the antibody portion. Procedures for generation of, for example, free sulfhydryl groups on polypeptides, such as antibodies or antibody fragments, are known (See U.S. Pat. No. 4,659,839).

Many procedures and linker molecules for attachment of various compounds including radionuclide metal chelates, toxins and drugs to proteins such as antibodies are known. See, for example, European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075. In particular, production of various immunotoxins is well-known within the art and can be found, for example in “Monoclonal Antibody-Toxin Conjugates: Aiming the Magic Bullet,” Thorpe et al., Monoclonal Antibodies in Clinical Medicine, Academic Press, pp. 168-190 (1982); Waldmann (1991) Science, 252: 1657; U.S. Pat. Nos. 4,545,985 and 4,894,443, and the like.

The term “linker” is used herein to denote polypeptides comprising one or more amino acid residues joined by peptide bonds and are used to link the TAA antibody and interferon molecules of the present disclosure. Generally the linker will have no specific biological activity other than to join the proteins or to preserve some minimum distance or other spatial relationship between them. In various embodiments, however, the constituent amino acids of the linker can be selected to influence some property of the molecule such as the folding, net charge, or hydrophobicity. In various embodiments, the linker is capable of forming covalent bonds to both the antibody and to the interferon. Suitable linkers are well known to those of skill in the art and include, but are not limited to, straight or branched-chain carbon linkers, heterocyclic carbon linkers, or peptide linkers. In certain embodiments, the linker(s) can be joined to the constituent amino acids of the antibody and/or the interferon through their side groups (e.g., through a disulfide linkage to cysteine). In certain preferred embodiments, the linkers are joined to the alpha carbon amino and/or carboxyl groups of the terminal amino acids of the antibody and/or the interferon. Such linker polypeptides are well known in the art (see e.g., Holliger, P., et al., Proc. Natl. Acad. Sci. (U.S.A.), 90:6444, 1993; Poljak, R. J., et al., Structure, 2:1121, 1994). Linker length contemplated for use can vary from about 5 to 200 amino acids.

In various embodiments, the linker is an α-helical linker. In various embodiments, the linker is rich in G/S content (e.g., at least about 60%, 70%, 80%, 90%, or more of the amino acids in the linker are G or S. In various embodiments, the linker is rich in G/C content and is less than about any of 2, 3, 4, 5, 6, 7, 8, 9, 10, 15, 20, 25, or 30 amino acid long. In various embodiments, the linker is an α-helical linker and is less than about any of 7, 8, 9, 10, 15, 20, 25, or 30 amino acid long. In various embodiments, the linker may be a proteolysis-resistant linker of 1 to 20 amino acids in length (see, e.g., U.S. Pat. No. 8,258,263 (Morrison et al.), hereby incorporated by reference in its entirety for the proteolysis-resistant linkers and sequences provided therein). In various embodiments, the linker is a proteolysis-resistant linker set forth in Table 4 below:

TABLE 4
Examples of Proteolysis-Resistant Linkers

| Linker Sequence | SEQ ID NO |
|---|---|
| SGGGGS | 18 |
| AEAAAKEAAAKAGS | 19 |
| GGGGS | 20 |
| SGGGGSGGGGS | 21 |
| GGGGG | 22 |
| GAGAGAGAGA | 23 |
| AEAAAKAGS | 24 |
| GGGGGGGG | 25 |
| AEAAAKEAAAKA | 26 |
| AEAAAKA | 27 |
| GGAGG | 28 |

In various embodiments, the linker comprises SGGGGS (SEQ ID NO: 18). In various embodiments, the linker comprises AEAAAKEAAAKAGS (SEQ ID NO: 19).

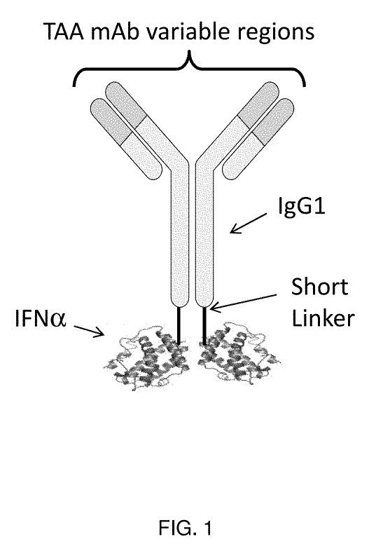

In various embodiments, the fusion molecule is a recombinantly expressed fusion molecule and will comprise interferon molecules attached to the antibody via a peptide linker as described herein and as depicted in FIG. 1. In various embodiments, the preparation of the TAA Ab-IFN fusion molecules of the present invention can be generally described as follows: the heavy chain of the TAA Ab is recombinantly engineered with an interferon, or mutant thereof, at the carboxy-terminus using a peptide linker. After verifying that the fusion protein containing vector has the correct nucleotide sequence, it is transfected, along with the vector containing the light chain into, e.g., CHO cells. Transfectants are screened by ELISA for the production of the complete fusion molecule. The clone giving the highest signal is expanded and following sub-cloning is grown in roller bottles. Conditioned medium is collected, concentrated, and the protein of interest purified using a single Protein A affinity chromatography step or appropriate alternative chromatography methods. The final product is formulated in a desired buffer and at a desired concentration (the protein concentration is confirmed by UV absorption). The purity of the final product is determined by SDS-PAGE both under reducing and non-reducing conditions. Western blot analysis is used to confirm the expected size of the molecule.

In various embodiments, the fusion molecules of the present disclosure will comprise the antibody, peptide linker, and interferon molecule combinations recited in Table 5.

TABLE 5
Examples of TAA Ab-IFN Fusion Molecules

| TAA Antibody | Peptide Linker | Interferons |
|---|---|---|
| Anti-HER2neu | SEQ ID NO: 18 or SEQ ID NO: 19 | wtIFN-α (SEQ ID NO: 1 or 2) |
| | | wtIFN-β (SEQ ID NO: 4 or 5) |
| Anti-CD20 | SEQ ID NO: 18 or SEQ ID NO: 19 | wtIFN-α (SEQ ID NO: 1 or 2) |
| | | wtIFN-β (SEQ ID NO: 4 or 5) |
| Anti-CD138 | SEQ ID NO: 18 or SEQ ID NO: 19 | wtIFN-α (SEQ ID NO: 1 or 2) |
| | | wtIFN-β (SEQ ID NO: 4 or 5) |
| Anti-endoplasmin | SEQ ID NO: 18 or SEQ ID NO: 19 | wtIFN-α (SEQ ID NO: 1 or 2) |
| | | wtIFN-β (SEQ ID NO: 4 or 5) |
| Anti-CD33 | SEQ ID NO: 18 or SEQ ID NO: 19 | wtIFN-α (SEQ ID NO: 1 or 2) |
| | | wtIFN-β (SEQ ID NO: 4 or 5) |
| Anti-CD70 | SEQ ID NO: 18 or SEQ ID NO: 19 | wtIFN-α (SEQ ID NO: 1 or 2) |
| | | wtIFN-β (SEQ ID NO: 4 or 5) |
| Anti-CD38 | SEQ ID NO: 18 or SEQ ID NO: 19 | wtIFN-α (SEQ ID NO: 1 or 2) |
| | | wtIFN-β (SEQ ID NO: 4 or 5) |
| Anti-BCMA | SEQ ID NO: 18 or SEQ ID NO: 19 | wtIFN-α (SEQ ID NO: 1 or 2) |
| | | wtIFN-β (SEQ ID NO: 4 or 5) |
| Anti-CD40 | SEQ ID NO: 18 or SEQ ID NO: 19 | wtIFN-α (SEQ ID NO: 1 or 2) |
| | | wtIFN-β (SEQ ID NO: 4 or 5) |
| Anti-CS1 | SEQ ID NO: 18 or SEQ ID NO: 19 | wtIFN-α (SEQ ID NO: 1 or 2) |
| | | wtIFN-β (SEQ ID NO: 4 or 5) |
| Anti-WT1 | SEQ ID NO: 18 or SEQ ID NO: 19 | wtIFN-α (SEQ ID NO: 1 or 2) |
| | | wtIFN-β (SEQ ID NO: 4 or 5) |
| Anti-B7H3 | SEQ ID NO: 18 or SEQ ID NO: 19 | wtIFN-α (SEQ ID NO: 1 or 2) |
| | | wtIFN-β (SEQ ID NO: 4 or 5) |
| Anti-PD-L1 | SEQ ID NO: 18 or SEQ ID NO: 19 | wtIFN-α (SEQ ID NO: 1 or 2) |
| | | wtIFN-β (SEQ ID NO: 4 or 5) |
| Anti-HER2neu | SEQ ID NO: 18 or SEQ ID NO: 19 | IFN-α Mutant (SEQ ID NO: 3) |
| Anti-CD20 | SEQ ID NO: 18 or SEQ ID NO: 19 | IFN-α Mutant (SEQ ID NO: 3) |
| Anti-CD138 | SEQ ID NO: 18 or SEQ ID NO: 19 | IFN-α Mutant (SEQ ID NO: 3) |
| Anti-endoplasmin | SEQ ID NO: 18 or SEQ ID NO: 19 | IFN-α Mutant (SEQ ID NO: 3) |
| Anti-CD33 | SEQ ID NO: 18 or SEQ ID NO: 19 | IFN-α Mutant (SEQ ID NO: 3) |
| Anti-CD70 | SEQ ID NO: 18 or SEQ ID NO: 19 | IFN-α Mutant (SEQ ID NO: 3) |
| Anti-CD38 | SEQ ID NO: 18 or SEQ ID NO: 19 | IFN-α Mutant (SEQ ID NO: 3) |
| Anti-BCMA | SEQ ID NO: 18 or SEQ ID NO: 19 | IFN-α Mutant (SEQ ID NO: 3) |
| Anti-CD40 | SEQ ID NO: 18 or SEQ ID NO: 19 | IFN-α Mutant (SEQ ID NO: 3) |
| Anti-CS1 | SEQ ID NO: 18 or SEQ ID NO: 19 | IFN-α Mutant (SEQ ID NO: 3) |
| Anti-WT1 | SEQ ID NO: 18 or SEQ ID NO: 19 | IFN-α Mutant (SEQ ID NO: 3) |
| Anti-B7H3 | SEQ ID NO: 18 or SEQ ID NO: 19 | IFN-α Mutant (SEQ ID NO: 3) |
| Anti-PD-L1 | SEQ ID NO: 18 or SEQ ID NO: 19 | IFN-α Mutant (SEQ ID NO: 3) |
| Anti-HER2neu | SEQ ID NO: 18 or SEQ ID NO: 19 | IFN-α Mutants M1-M15 |
| Anti-CD20 | SEQ ID NO: 18 or SEQ ID NO: 19 | IFN-α Mutants M1-M15 |
| Anti-CD138 | SEQ ID NO: 18 or SEQ ID NO: 19 | IFN-α Mutants M1-M15 |
| Anti-endoplasmin | SEQ ID NO: 18 or SEQ ID NO: 19 | IFN-α Mutants M1-M15 |
| Anti-CD33 | SEQ ID NO: 18 or SEQ ID NO: 19 | IFN-α Mutants M1-M15 |
| Anti-CD70 | SEQ ID NO: 18 or SEQ ID NO: 19 | IFN-α Mutants M1-M15 |
| Anti-CD38 | SEQ ID NO: 18 or SEQ ID NO: 19 | IFN-α Mutants M1-M15 |
| Anti-BCMA | SEQ ID NO: 18 or SEQ ID NO: 19 | IFN-α Mutants M1-M15 |
| Anti-CD40 | SEQ ID NO: 18 or SEQ ID NO: 19 | IFN-α Mutants M1-M15 |
| Anti-CS1 | SEQ ID NO: 18 or SEQ ID NO: 19 | IFN-α Mutants M1-M15 |
| Anti-WT1 | SEQ ID NO: 18 or SEQ ID NO: 19 | IFN-α Mutants M1-M15 |
| Anti-B7H3 | SEQ ID NO: 18 or SEQ ID NO: 19 | IFN-α Mutants M1-M15 |
| Anti-PD-L1 | SEQ ID NO: 18 or SEQ ID NO: 19 | IFN-α Mutants M1-M15 |
| Anti-HER2neu | SEQ ID NO: 18 or SEQ ID NO: 19 | Truncated IFN-β's IFN-Δ1-Δ10 |
| | | US 2009/0025106 |
| Anti-CD20 | SEQ ID NO: 18 or SEQ ID NO: 19 | Truncated IFN-β's IFN-Δ1-Δ10 |
| | | US 2009/0025106 |
| Anti-CD138 | SEQ ID NO: 18 or SEQ ID NO: 19 | Truncated IFN-β's IFN-Δ1-Δ10 |
| | | US 2009/0025106 |
| Anti-endoplasmin | SEQ ID NO: 18 or SEQ ID NO: 19 | Truncated IFN-β's IFN-Δ1-Δ10 |
| | | US 2009/0025106 |
| Anti-CD33 | SEQ ID NO: 18 or SEQ ID NO: 19 | Truncated IFN-β's IFN-Δ1-Δ10 |
| | | US 2009/0025106 |
| Anti-CD70 | SEQ ID NO: 18 or SEQ ID NO: 19 | Truncated IFN-β's IFN-Δ1-Δ10 |
| | | US 2009/0025106 |
| Anti-CD38 | SEQ ID NO: 18 or SEQ ID NO: 19 | Truncated IFN-β's IFN-Δ1-Δ10 |
| | | US 2009/0025106 |
| Anti-BCMA | SEQ ID NO: 18 or SEQ ID NO: 19 | Truncated IFN-β's IFN-Δ1-Δ10 |
| | | US 2009/0025106 |
| Anti-CD40 | SEQ ID NO: 18 or SEQ ID NO: 19 | Truncated IFN-β's IFN-Δ1-Δ10 |
| | | US 2009/0025106 |
| Anti-CS1 | SEQ ID NO: 18 or SEQ ID NO: 19 | Truncated IFN-β's IFN-Δ1-Δ10 |
| | | US 2009/0025106 |
| Anti-WT1 | SEQ ID NO: 18 or SEQ ID NO: 19 | Truncated IFN-β's IFN-Δ1-Δ10 |
| | | US 2009/0025106 |
| Anti-B7H3 | SEQ ID NO: 18 or SEQ ID NO: 19 | Truncated IFN-β's IFN-Δ1-Δ10 |
| | | US 2009/0025106 |
| Anti-PD-L1 | SEQ ID NO: 18 or SEQ ID NO: 19 | Truncated IFN-β's IFN-Δ1-Δ10 |
| | | US 2009/0025106 |

### Pharmaceutical Compositions

In another aspect, the present disclosure provides a pharmaceutical composition comprising a fusion molecule as described herein, and a second anti-cancer agent, with one or more pharmaceutically acceptable excipient(s). The pharmaceutical compositions and methods of uses described herein also encompass embodiments of combinations (co-administration) with other active agents, as detailed below. The fusion molecules provided herein can be formulated by a variety of methods apparent to those of skill in the art of pharmaceutical formulation. Such methods may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration.

Generally, fusion molecules of the invention are suitable to be administered as a formulation in association with one or more pharmaceutically acceptable excipient(s), or carriers. Such pharmaceutically acceptable excipients and carriers are well known and understood by those of ordinary skill and have been extensively described (see, e.g., Remington's Pharmaceutical Sciences, 18th Edition, A. R. Gennaro, ed., Mack Publishing Company, 1990). The pharmaceutically acceptable carriers may be included for purposes of modifying, maintaining or preserving, for example, the pH, osmolarity, viscosity, clarity, color, isotonicity, odor, sterility, stability, rate of dissolution or release, adsorption or penetration of the composition. Such pharmaceutical compositions may influence the physical state, stability, rate of in vivo release, and rate of in vivo clearance of the polypeptide. Suitable pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

The pharmaceutical compositions of the invention are typically suitable for parenteral administration. As used herein, “parenteral administration” of a pharmaceutical composition includes any route of administration characterized by physical breaching of a tissue of a patient and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In various embodiments, the pharmaceutical composition is formulated for parenteral administration via a route selected from, e.g., subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or via infusions.

When parenteral administration is contemplated, the therapeutic pharmaceutical compositions may be in the form of a pyrogen-free, parenterally acceptable aqueous solution comprising the desired fusion molecule in a pharmaceutically acceptable vehicle. A particularly suitable vehicle for parenteral injection is sterile distilled water in which a polypeptide is formulated as a sterile, isotonic solution, properly preserved. In various embodiments, pharmaceutical formulations suitable for injectable administration may be formulated in aqueous solutions, preferably in physiologically compatible buffers such as Hanks' solution, Ringer's solution, or physiologically buffered saline. Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Optionally, the suspension may also contain suitable stabilizers or agents to increase the solubility of the compounds and allow for the preparation of highly concentrated solutions. Injectable formulations may be prepared, packaged, or sold in unit dosage form, such as in ampoules or in multi-dose containers containing a preservative. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

Any method for formulating and administering peptides, proteins, antibodies, and immunoconjugates accepted in the art may suitably be employed for administering the fusion molecules of the present invention.

### Dosing

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form,” as used herein, refers to physically discrete units suited as unitary dosages for the subjects to be treated; each unit contains a predetermined quantity of active compound calculated to produce the desired therapeutic effect in association with the required pharmaceutical carrier.

The precise dose of fusion molecule to be employed in the methods of the present disclosure will depend on the route of administration, and the seriousness of the disease or disorder, and should be decided according to the judgment of the practitioner and each subject's circumstances. It is to be noted that dosage values may include single or multiple doses, and that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

For administration to human subjects, the total monthly dose of the fusion molecules of the invention can be in the range of 0.002-500 mg per patient, 0.002-400 mg per patient, 0.002-300 mg per patient, 0.002-200 mg per patient, 0.002-100 mg per patient, 0.002-50 mg per patient, 0.006-500 mg per patient, 0.006-400 mg per patient, 0.006-300 mg per patient, 0.006-200 mg per patient, 0.006-100 mg per patient, 0.006-50 mg per patient, 0.02-500 mg per patient, 0.02-400 mg per patient, 0.02-300 mg per patient, 0.02-200 mg per patient, 0.02-100 mg per patient, 0.02-50 mg per patient, 0.06-500 mg per patient, 0.06-400 mg per patient, 0.06-300 mg per patient, 0.06-200 mg per patient, 0.06-100 mg per patient, 0.06-50 mg per patient, 0.2-500 mg per patient, 0.2-400 mg per patient, 0.2-300 mg per patient, 0.2-200 mg per patient, 0.2-100 mg per patient, 0.2-50 mg per patient, 0.6-500 mg per patient, 0.6-400 mg per patient, 0.6-300 mg per patient, 0.6-200 mg per patient, 0.6-100 mg per patient, or 0.6-50 mg per patient, 2-500 mg per patient, 2-400 mg per patient, 2-300 mg per patient, 2-200 mg per patient, 2-100 mg per patient, 2-50 mg per patient, 6-500 mg per patient, 6-400 mg per patient, 6-300 mg per patient, 6-200 mg per patient, 6-100 mg per patient, or 6-50 mg per patient, depending, of course, on the mode of administration. The total monthly dose can be administered in single or divided doses and can, at the physician's discretion, fall outside of the typical ranges given herein.

An exemplary, non-limiting weekly dosing range for a therapeutically effective amount of the fusion molecules of the invention can be about 0.0001 to about 0.9 mg/kg, about 0.0001 to about 0.8 mg/kg, about 0.0001 to about 0.7 mg/kg, about 0.0001 to about 0.6 mg/kg, about 0.0001 to about 0.5 mg/kg, about 0.0001 to about 0.4 mg/kg, about 0.0001 to about 0.3 mg/kg, about 0.0001 to about 0.2 mg/kg, about 0.0001 to about 0.1 mg/kg, about 0.0003 to about 0.9 mg/kg, about 0.0003 to about 0.8 mg/kg, about 0.0003 to about 0.7 mg/kg, about 0.0003 to about 0.6 mg/kg, about 0.0003 to about 0.5 mg/kg, about 0.0003 to about 0.4 mg/kg, about 0.0003 to about 0.3 mg/kg, about 0.0003 to about 0.2 mg/kg, about 0.0003 to about 0.1 mg/kg, about 0.001 to about 0.9 mg/kg, about 0.001 to about 0.8 mg/kg, about 0.001 to about 0.7 mg/kg, about 0.001 to about 0.6 mg/kg, about 0.001 to about 0.5 mg/kg, about 0.001 to about 0.4 mg/kg, about 0.001 to about 0.3 mg/kg, about 0.001 to about 0.2 mg/kg, about 0.0001 to about 0.1 mg/kg, about 0.003 to about 0.9 mg/kg, about 0.003 to about 0.8 mg/kg, about 0.003 to about 0.7 mg/kg, about 0.003 to about 0.6 mg/kg, about 0.003 to about 0.5 mg/kg, about 0.003 to about 0.4 mg/kg, about 0.003 to about 0.3 mg/kg, about 0.003 to about 0.2 mg/kg, about 0.003 to about 0.1 mg/kg, about 0.01 to about 0.9 mg/kg, about 0.01 to about 0.8 mg/kg, about 0.01 to about 0.7 mg/kg, about 0.01 to about 0.6 mg/kg, about 0.01 to about 0.5 mg/kg, about 0.01 to about 0.4 mg/kg, about 0.01 to about 0.3 mg/kg, about 0.01 to about 0.2 mg/kg, about 0.01 to about 0.1 mg/kg, about 0.03 to about 0.9 mg/kg, about 0.03 to about 0.8 mg/kg, about 0.03 to about 0.7 mg/kg, about 0.03 to about 0.6 mg/kg, about 0.03 to about 0.5 mg/kg, about 0.03 to about 0.4 mg/kg, about 0.03 to about 0.3 mg/kg, about 0.03 to about 0.2 mg/kg, about 0.03 to about 0.1 mg/kg, about 0.1 to about 0.9 mg/kg, about 0.1 to about 0.8 mg/kg, about 0.1 to about 0.7 mg/kg, about 0.1 to about 0.6 mg/kg, about 0.1 to about 0.5 mg/kg, about 0.1 to about 0.4 mg/kg, about 0.1 to about 0.3 mg/kg, about 0.1 to about 0.2 mg/kg, about 0.1 to about 0.1 mg/kg, about 0.3 to about 0.9 mg/kg, about 0.3 to about 0.8 mg/kg, about 0.3 to about 0.7 mg/kg, about 0.3 to about 0.6 mg/kg, about 0.3 to about 0.5 mg/kg, about 0.3 to about 0.4 mg/kg, about 0.3 to about 0.3 mg/kg, about 0.3 to about 0.2 mg/kg, about 0.3 to about 0.1 mg/kg.

In various embodiments, the TAA Ab-IFN fusion molecule is administered to the patient at a weekly dosage selected from the group consisting of no greater than 0.0001 mg/kg, no greater than 0.0003 mg/kg, no greater than 0.001 mg/kg, no greater than 0.003 mg/kg, no greater than 0.01 mg/kg, no greater than 0.03 mg/kg, no greater than 0.1 mg/kg, no greater than 0.2 mg/kg, no greater than 0.3 mg/kg, no greater than 0.4 mg/kg, no greater than 0.5 mg/kg, no greater than 0.6 mg/kg, no greater than 0.7 mg/kg, no greater than 0.8 mg/kg, and no greater than 0.9 mg/kg.

In various embodiments, the weekly dose for a therapeutically effective amount of a fusion molecule of the invention will be 0.0001 mg/kg body weight. In various embodiments, the weekly dose for a therapeutically effective amount of a fusion molecule of the invention will be 0.0003 mg/kg body weight. In various embodiments, the weekly dose for a therapeutically effective amount of a fusion molecule of the invention will be 0.001 mg/kg body weight. In various embodiments, the weekly dose for a therapeutically effective amount of a fusion molecule of the invention will be 0.003 mg/kg body weight. In various embodiments, the weekly dose for a therapeutically effective amount of a fusion molecule of the invention will be 0.01 mg/kg body weight. In various embodiments, the weekly dose for a therapeutically effective amount of a fusion molecule of the invention will be 0.03 mg/kg body weight. In various embodiments, the weekly dose for a therapeutically effective amount of a fusion molecule of the invention will be 0.1 mg/kg body weight. In various embodiments, the weekly dose for a therapeutically effective amount of a fusion molecule of the invention will be 0.3 mg/kg body weight. In various embodiments the fusion molecules will be administered via intravenous (IV) infusion for up to three cycles of eight once weekly doses.

In various embodiments, single or multiple administrations of the pharmaceutical compositions are administered depending on the dosage and frequency as required and tolerated by the patient. In any event, the composition should provide a sufficient quantity of at least one of the fusion molecules disclosed herein to effectively treat the patient. The dosage can be administered once but may be applied periodically until either a therapeutic result is achieved or until side effects warrant discontinuation of therapy.

The dosing frequency of the administration of the fusion molecule pharmaceutical composition depends on the nature of the therapy and the particular disease being treated. The patient can be treated at regular intervals, such as weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, twice weekly without break for 2 weeks, twice weekly without break for 3 weeks, twice weekly without break for 4 weeks, twice weekly without break for 5 weeks, twice weekly without break for 6 weeks, twice weekly without break for 7 weeks, twice weekly without break for 8 weeks, monthly; once every other month; once every three months; once every four months; once every five months; or once every six months, or yearly.

### TAA Ab-IFN Fusion Molecule Therapeutic Methods of Use

In one aspect, the present invention relates to a method of treating a proliferative disease (such as cancer) in an individual, comprising administering to the individual a therapeutically effective amount of a TAA Ab-IFN fusion molecule. Importantly, the TAA Ab-IFN fusion molecules and methods described herein can be used to effectively treat cancers, including recurrent, resistant, or refractory cancers, at surprisingly low doses.

In various embodiments, the methods of the present invention are useful in treating certain cellular proliferative diseases. Such diseases include, but are not limited to, the following: a) proliferative diseases of the breast, which include, but are not limited to, invasive ductal carcinoma, invasive lobular carcinoma, ductal carcinoma, lobular carcinoma in situ and metastatic breast cancer; b) proliferative diseases of lymphocytic cells, which include, but are not limited to, various T cell and B cell lymphomas, non-Hodgkins lymphoma, cutaneous T cell lymphoma, Hodgkins disease, and lymphoma of the central nervous system; (c) multiple myeloma, chronic neutrophilic leukemia, chronic eosinophilic leukemia/hypereosinophilic syndrome, chronic idiopathic myelofibrosis, polycythemia vera, essential thrombocythemia, chronic myelomonocytic leukemia, atypical chronic myelogenous leukemia, juvenile myelomonocytic leukemia, refractory anemia with ringed sideroblasts and without ringed sideroblasts, refractory cytopenia (myelodysplastic syndrome) with multilineage dysplasia, refractory anemia (myelodysplastic syndrome) with excess blasts, 5q-syndrome, myelodysplastic syndrome with t(9;12)(q22;p12), and myelogenous leukemia (e.g., Philadelphia chromosome positive (t(9;22)(qq34;q11)); d) proliferative diseases of the skin, which include, but are not limited to, basal cell carcinoma, squamous cell carcinoma, malignant melanoma and Kaposi's sarcoma; e) leukemias, which include, but are not limited to, acute myeloid leukemia, acute lymphoblastic leukemia, chronic lymphocytic leukemia, chronic myelogenous leukemia, and hairy cell leukemia, f) proliferative diseases of the digestive tract, which include, but are not limited to, anal, colon, colorectal, esophageal, gallbladder, stomach (gastric), pancreatic cancer, pancreatic cancer-Islet cell, rectal, small-intestine and salivary gland cancers; g) proliferative diseases of the liver, which include, but are not limited to, hepatocellular carcinoma, cholangiocarcinoma, mixed hepatocellular cholangiocarcinoma, primary liver cancer and metastatic liver cancer; h) proliferative diseases of the male reproductive organs, which include, but are not limited to, prostate cancer, testicular cancer and penile cancer; i) proliferative diseases of the female reproductive organs, which include, but are not limited to, uterine cancer (endometrial), cervical, ovarian, vaginal, vulval cancers, uterine sarcoma and ovarian germ cell tumor; j) proliferative diseases of the respiratory tract, which include, but are not limited to, small cell and non-small cell lung carcinoma, bronchial adema, pleuropulmonary blastoma and malignant mesothelioma; k) proliferative diseases of the brain, which include, but are not limited to, brain stem and hyptothalamic glioma, cerebellar and cerebral astrocytoma, medullablastoma, ependymal tumors, oligodendroglial, meningiomas and neuroectodermal and pineal tumors; l) proliferative diseases of the eye, which include, but are not limited to, intraocular melanoma, retinoblastoma, and rhabdomyosarcoma; m) proliferative diseases of the head and neck, which include, but are not limited to, laryngeal, hypopharyngeal, nasopharyngeal, oropharyngeal cancers, and lip and oral cancer, squamous neck cancer, metastatic paranasal sinus cancer; n) proliferative diseases of the thyroid, which include, but are not limited to, thyroid cancer, thymoma, malignant thymoma, medullary thyroid carcinomas, papillary thyroid carcinomas, multiple endocrine neoplasia type 2A (MEN2A), pheochromocytoma, parathyroid adenomas, multiple endocrine neoplasia type 2B (MEN2B), familial medullary thyroid carcinoma (FMTC) and carcinoids; o) proliferative diseases of the urinary tract, which include, but are not limited to, bladder cancer; p) sarcomas, which include, but are not limited to, sarcoma of the soft tissue, osteosarcoma, malignant fibrous histiocytoma, lymphosarcoma, and rhabdomyosarcoma; q) proliferative diseases of the kidneys, which include, but are not limited to, renal cell carcinoma, clear cell carcinoma of the kidney; and renal cell adenocarcinoma; r) precursor B-lymphoblastic leukemia/lymphoma (precursor B-cell acute lymphoblastic leukemia), B-cell chronic lymphocytic leukemia/small lymphocytic lymphoma, B-cell prolymphocytic leukemia, lymphoplasmacytic lymphoma, splenic marginal zone B-cell lymphoma, hairy cell leukemia, plasma cell myeloma/plasmacytoma, extranodal marginal zone B-cell lymphoma of MALT type, nodal marginal zone B-cell lymphoma, follicular lymphoma, mantle-cell lymphoma, diffuse large B-cell lymphoma, mediastinal large B-cell lymphoma, primary effusion lymphoma and Burkitt's lymphoma/Burkitt cell leukemia; (s) precursor T-lymphoblastic lymphoma/leukemia (precursor T-cell acute lymphoblastic leukemia), T-cell prolymphocytic leukemia, T-cell granular lymphocytic leukemia, aggressive NK-cell leukemia, adult T-cell lymphoma/leukemia (HTLV-1), extranodal NK/T-cell lymphoma, nasal type, enteropathy-type T-cell lymphoma, hepatosplenic gamma-delta T-cell lymphoma, subcutaneous panniculitis-like T-cell lymphoma, mycosis fungoides/Sezary syndrome, anaplastic large-cell lymphoma, T/null cell, primary cutaneous type, peripheral T-cell lymphoma, not otherwise characterized, angioimmunoblastic T-cell lymphoma, anaplastic large-cell lymphoma, T/null cell, and primary systemic type; (t) nodular lymphocyte-predominant Hodgkin's lymphoma, nodular sclerosis Hodgkin's lymphoma (grades 1 and 2), lymphocyte-rich classical Hodgkin's lymphoma, mixed cellularity Hodgkin's lymphoma, and lymphocyte depletion Hodgkin's lymphoma; and (u) AML with t(8;21)(q22;q22), AML1(CBF-alpha)/ETO, acute promyelocytic leukemia (AML with t(15;17)(q22;q11-12) and variants, PML/RAR-alpha), AML with abnormal bone marrow eosinophils (inv(16)(p13q22) or t(16;16)(p13;q11), CBFb/MYH11.times.), and AML with 11q23 (MLL) abnormalities, AML minimally differentiated, AML without maturation, AML with maturation, acute myelomonocytic leukemia, acute monocytic leukemia, acute erythroid leukemia, acute megakaryocytic leukemia, acute basophilic leukemia, and acute panmyelosis with myelofibrosis.

In various embodiments, the proliferative disease is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In various embodiments, there is provided a method of treating a cancer in an individual, comprising administering to the individual an effective amount of a pharmaceutical composition comprising an anti-TAA Ab-IFN-α fusion molecule, wherein the anti-TAA-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-TAA-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-TAA-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses the TAA of the anti-TAA Ab-IFN-α fusion molecule of the present invention. In various embodiments, the cancer is a non-TAA expressing cancer in the tumor microenvironment of a TAA expressing cancer.

In various embodiments, there is provided a method of treating a cancer in an individual, comprising administering to the individual an effective amount of a pharmaceutical composition comprising an anti-TAA Ab-IFN-α fusion molecule, wherein the TAA Ab-IFN-α fusion molecule is administered to the individual at a weekly dosage of about 0.003 to about 0.01 mg/kg.

In various embodiments, there is provided a method of treating a cancer in an individual, comprising administering to the individual an effective amount of a pharmaceutical composition comprising an anti-TAA Ab-IFN-α fusion molecule, wherein the TAA Ab-IFN-α fusion molecule is administered to the individual at a weekly dosage of about 0.01 to about 0.03 mg/kg.

In various embodiments, there is provided a method of treating a cancer in an individual, comprising administering to the individual an effective amount of a pharmaceutical composition comprising an anti-TAA Ab-IFN-α fusion molecule, wherein the TAA Ab-IFN-α fusion molecule is administered to the individual at a weekly dosage of about 0.03 to about 0.1 mg/kg.

In various embodiments, there is provided a method of treating a cancer in an individual, comprising administering to the individual an effective amount of a pharmaceutical composition comprising an anti-HER2/neu-IFN-α fusion molecule, wherein the anti-HER2/neu-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-HER2/neu-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-HER2/neu-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses HER2/neu. In various embodiments, the cancer is a non-HER2/neu expressing cancer in the tumor microenvironment of a HER2/neu expressing cancer.

In various embodiments, there is provided a method of treating a cancer selected from the group consisting of breast cancer, ovarian cancer and non-small cell lung cancer (NSCLC) in an individual, comprising administering to the individual an effective amount of a pharmaceutical composition comprising an anti-HER2/neu Ab-IFN-α fusion molecule, wherein the anti-HER2/neu Ab-IFN fusion-α molecule is administered to the individual at a weekly dosage of about 0.003 to about 0.01 mg/kg.

In various embodiments, there is provided a method of treating a cancer selected from the group consisting of breast cancer, ovarian cancer and non-small cell lung cancer (NSCLC) in an individual, comprising administering to the individual an effective amount of a pharmaceutical composition comprising an anti-HER2/neu Ab-IFN-α fusion molecule, wherein the anti-HER2/neu Ab-IFN-α fusion molecule is administered to the individual at a weekly dosage of about 0.01 to about 0.03 mg/kg.

In various embodiments, there is provided a method of treating a cancer selected from the group consisting of breast cancer, ovarian cancer and non-small cell lung cancer (NSCLC) in an individual, comprising administering to the individual an effective amount of a pharmaceutical composition comprising an anti-HER2/neu Ab-IFN-α fusion molecule, wherein the anti-HER2/neu Ab-IFN-α fusion molecule is administered to the individual at a weekly dosage of about 0.03 to about 0.1 mg/kg.

In various embodiments, there is provided a method of treating a cancer in an individual, comprising administering to the individual an effective amount of a pharmaceutical composition comprising an anti-CD20-IFN-α fusion molecule, wherein the anti-CD20-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-CD20-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-CD20-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses CD20. In various embodiments, the cancer is a non-CD20 expressing cancer in the tumor microenvironment of a CD20 expressing cancer.

In various embodiments, there is provided a method of treating a cancer selected from the group consisting of B-cell Non-Hodgkin's lymphoma (NHL) and B-cell chronic lymphocytic leukemia (CLL) in an individual, comprising administering to the individual an effective amount of a pharmaceutical composition comprising an anti-CD20 Ab-IFN-α fusion molecule, wherein the anti-CD20 Ab-IFN-α fusion molecule is administered to the individual at a weekly dosage of about 0.003 to about 0.01 mg/kg.

In various embodiments, there is provided a method of treating a cancer selected from the group consisting of B-cell Non-Hodgkin's lymphoma (NHL) and B-cell chronic lymphocytic leukemia (CLL) in an individual, comprising administering to the individual an effective amount of a pharmaceutical composition comprising an anti-CD20 Ab-IFN-α fusion molecule, wherein the anti-CD20 Ab-IFN-α fusion molecule is administered to the individual at a weekly dosage of about 0.01 to about 0.03 mg/kg.

In various embodiments, there is provided a method of treating a cancer selected from the group consisting of B-cell Non-Hodgkin's lymphoma (NHL) and B-cell chronic lymphocytic leukemia (CLL) in an individual, comprising administering to the individual an effective amount of a pharmaceutical composition comprising an anti-CD20 Ab-IFN-α fusion molecule, wherein the anti-CD20 Ab-IFN-α fusion molecule is administered to the individual at a weekly dosage of about 0.03 to about 0.1 mg/kg.

In various embodiments, there is provided a method of treating a cancer in an individual, comprising administering to the individual an effective amount of a pharmaceutical composition comprising an anti-CD138-IFN-α fusion molecule, wherein the anti-CD138-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-CD138-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-CD138-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses CD138. In various embodiments, the cancer is a non-CD138 expressing cancer in the tumor microenvironment of a CD138 expressing cancer.

In various embodiments, there is provided a method of treating a cancer selected from the group consisting of multiple myeloma, breast cancer, and bladder cancer in an individual, comprising administering to the individual an effective amount of a pharmaceutical composition comprising an anti-CD138 Ab-IFN-α fusion molecule, wherein the anti-CD138 Ab-IFN-α fusion molecule is administered to the individual at a weekly dosage of about 0.003 to about 0.01 mg/kg.

In various embodiments, there is provided a method of treating a cancer selected from the group consisting of multiple myeloma, breast cancer, and bladder cancer in an individual, comprising administering to the individual an effective amount of a pharmaceutical composition comprising an anti-CD138 Ab-IFN-α fusion molecule, wherein the anti-CD138 Ab-IFN-α fusion molecule is administered to the individual at a weekly dosage of about 0.01 to about 0.03 mg/kg.

In various embodiments, there is provided a method of treating a cancer selected from the group consisting of multiple myeloma, breast cancer, and bladder cancer in an individual, comprising administering to the individual an effective amount of a pharmaceutical composition comprising an anti-CD138 Ab-IFN-α fusion molecule, wherein the anti-CD138 Ab-IFN-α fusion molecule is administered to the individual at a weekly dosage of about 0.03 to about 0.1 mg/kg.

In various embodiments, there is provided a method of treating a cancer in an individual, comprising administering to the individual an effective amount of a pharmaceutical composition comprising an anti-GRP94-IFN-α fusion molecule, wherein the anti-GRP94-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-GRP94-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-GRP94-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses GRP94. In various embodiments, the cancer is a non-GRP94 expressing cancer in the tumor microenvironment of a GRP94 expressing cancer.

In various embodiments, there is provided a method of treating a cancer selected from the group consisting of NSCLC, acute myeloid leukemia (AML), multiple myeloma, melanoma, and pancreatic cancer in an individual, comprising administering to the individual an effective amount of a pharmaceutical composition comprising an anti-GRP94 Ab-IFN-α fusion molecule, wherein the anti-GRP94 Ab-IFN-α fusion molecule is administered to the individual at a weekly dosage of about 0.003 to about 0.01 mg/kg.

In various embodiments, there is provided a method of treating a cancer selected from the group consisting of NSCLC, acute myeloid leukemia (AML), multiple myeloma, melanoma, and pancreatic cancer in an individual, comprising administering to the individual an effective amount of a pharmaceutical composition comprising an anti-GRP94 Ab-IFN-α fusion molecule, wherein the anti-GRP94 Ab-IFN-α fusion molecule is administered to the individual at a weekly dosage of about 0.01 to about 0.03 mg/kg.

In various embodiments, there is provided a method of treating a cancer selected from the group consisting of NSCLC, acute myeloid leukemia (AML), multiple myeloma, melanoma, and pancreatic cancer in an individual, comprising administering to the individual an effective amount of a pharmaceutical composition comprising an anti-GRP94 Ab-IFN-α fusion molecule, wherein the anti-GRP94 Ab-IFN-α fusion molecule is administered to the individual at a weekly dosage of about 0.03 to about 0.1 mg/kg.

In various embodiments, there is provided a method of treating a cancer in an individual, comprising administering to the individual an effective amount of a pharmaceutical composition comprising an anti-CD33-IFN-α fusion molecule, wherein the anti-CD33-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-CD33-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-CD33-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses CD33. In various embodiments, the cancer is a non-CD33 expressing cancer in the tumor microenvironment of a CD33 expressing cancer.

In various embodiments, there is provided a method of treating a cancer selected from the group consisting of AML, chronic myeloid leukemia (CML) and multiple myeloma in an individual, comprising administering to the individual an effective amount of a pharmaceutical composition comprising an anti-CD33 Ab-IFN-α fusion molecule, wherein the anti-CD33 Ab-IFN-α fusion molecule is administered to the individual at a weekly dosage of about 0.003 to about 0.01 mg/kg.

In various embodiments, there is provided a method of treating a cancer selected from the group consisting of AML, chronic myeloid leukemia (CML) and multiple myeloma in an individual, comprising administering to the individual an effective amount of a pharmaceutical composition comprising an anti-CD33 Ab-IFN-α fusion molecule, wherein the anti-CD33 Ab-IFN-α fusion molecule is administered to the individual at a weekly dosage of about 0.01 to about 0.03 mg/kg.

In various embodiments, there is provided a method of treating a cancer selected from the group consisting of AML, chronic myeloid leukemia (CML) and multiple myeloma in an individual, comprising administering to the individual an effective amount of a pharmaceutical composition comprising an anti-CD33 Ab-IFN-α fusion molecule, wherein the anti-CD33 Ab-IFN-α fusion molecule is administered to the individual at a weekly dosage of about 0.03 to about 0.1 mg/kg.

In various embodiments, there is provided a method of treating a cancer in an individual, comprising administering to the individual an effective amount of a pharmaceutical composition comprising an anti-CD70-IFN-α fusion molecule, wherein the anti-CD70-IFN-α fusion molecule is administered to the individual at a weekly dosage selected from the group consisting of 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the anti-CD70-IFN-α fusion molecule is administered to the individual at a dosage (e.g., at a weekly dosage) included in any of the following ranges: about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the anti-CD70-IFN-α fusion molecule is administered to the individual at a dosage (e.g., at a weekly dosage) of no greater than about any of: 0.0001 mg/kg, 0.0003 mg/kg, 0.001 mg/kg, 0.003 mg/kg, 0.01 mg/kg, 0.03 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, and 0.9 mg/kg. In various embodiments, the cancer expresses CD70. In various embodiments, the cancer is a non-CD70 expressing cancer in the tumor microenvironment of a CD70 expressing cancer.

In various embodiments, there is provided a method of treating a cancer selected from the group consisting of renal cell carcinoma (RCC), Waldenstrom macroglobulinemia, multiple myeloma, and NHL in an individual, comprising administering to the individual an effective amount of a pharmaceutical composition comprising an anti-CD70 Ab-IFN-α fusion molecule, wherein the anti-CD70 Ab-IFN-α fusion molecule is administered to the individual at a weekly dosage of about 0.003 to about 0.01 mg/kg.

In various embodiments, there is provided a method of treating a cancer selected from the group consisting of renal cell carcinoma (RCC), Waldenstrom macroglobulinemia, multiple myeloma, and NHL in an individual, comprising administering to the individual an effective amount of a pharmaceutical composition comprising an anti-CD70 Ab-IFN-α fusion molecule, wherein the anti-CD70 Ab-IFN-α fusion molecule is administered to the individual at a weekly dosage of about 0.01 to about 0.03 mg/kg.

In various embodiments, there is provided a method of treating a cancer selected from the group consisting of renal cell carcinoma (RCC), Waldenstrom macroglobulinemia, multiple myeloma, and NHL in an individual, comprising administering to the individual an effective amount of a pharmaceutical composition comprising an anti-CD70 Ab-IFN-α fusion molecule, wherein the anti-CD70 Ab-IFN-α fusion molecule is administered to the individual at a weekly dosage of about 0.03 to about 0.1 mg/kg.

In various embodiments, the individual previously responded to treatment with an anti-cancer therapy, but, upon cessation of therapy, suffered relapse (hereinafter “a recurrent cancer”).

In various embodiments, the individual has resistant or refractory cancer. In various embodiments, the cancer is refractory to immunotherapy treatment. In various embodiments, the cancer is refractory to treatment with a chemotherapeutic agent. In various embodiments, the cancer is refractory to targeted treatment with a TAA Ab. In various embodiments, the cancer is refractory to targeted treatment with an immunoconjugate, antibody-drug conjugate (ADC), or fusion molecule comprising a TAA Ab and a cytotoxic agent. In various embodiments, the cancer is refractory to targeted treatment with a small molecule kinase inhibitor. In various embodiments, the cancer is refractory to combination therapy involving, e.g, immunotherapy, treatment with a chemotherapeutic agent, treatment with a TAA Ab, treatment with a immunoconjugate, ADC, or fusion molecule comprising a TAA Ab and a cytotoxic agent, targeted treatment with a small molecule kinase inhibitor, treatment using surgery, treatment using stem cell transplantation, and treatment using radiation.

In various embodiments, the methods described herein may be used in combination with other conventional anti-cancer therapeutic approaches directed to treatment or prevention of proliferative disorders, such approaches including, but not limited to chemotherapy, small molecule kinase inhibitor targeted therapy, surgery, radiation therapy, and stem cell transplantation. For example, such methods can be used in prophylactic cancer prevention, prevention of cancer recurrence and metastases after surgery, and as an adjuvant of other conventional cancer therapy. The present disclosure recognizes that the effectiveness of conventional cancer therapies (e.g., chemotherapy, radiation therapy, phototherapy, immunotherapy, and surgery) can be enhanced through the use of the fusion molecules described herein.

A wide array of conventional compounds has been shown to have anti-neoplastic activities. These compounds have been used as pharmaceutical agents in chemotherapy to shrink solid tumors, prevent metastases and further growth, or decrease the number of malignant T-cells in leukemic or bone marrow malignancies. Although chemotherapy has been effective in treating various types of malignancies, many anti-neoplastic compounds induce undesirable side effects. It has been shown that when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

When the TAA Ab-IFN fusion molecule disclosed herein is administered in combination with another conventional anti-neoplastic agent, either concomitantly or sequentially, such fusion molecule may enhance the therapeutic effect of the anti-neoplastic agent or overcome cellular resistance to such anti-neoplastic agent. This allows decrease of dosage of an anti-neoplastic agent, thereby reducing the undesirable side effects, or restores the effectiveness of an anti-neoplastic agent in resistant T-cells. In various embodiments, a second anti-cancer agent, such as a chemotherapeutic agent, will be administered to the patient. The list of exemplary chemotherapeutic agent includes, but is not limited to, daunorubicin, dactinomycin, doxorubicin, bleomycin, mitomycin, nitrogen mustard, chlorambucil, melphalan, cyclophosphamide, 6-mercaptopurine, 6-thioguanine, bendamustine, cytarabine (CA), 5-fluorouracil (5-FU), floxuridine (5-FUdR), methotrexate (MTX), colchicine, vincristine, vinblastine, etoposide, teniposide, cisplatin, carboplatin, oxaliplatin, pentostatin, cladribine, cytarabine, gemcitabine, pralatrexate, mitoxantrone, diethylstilbestrol (DES), fluradabine, ifosfamide, hydroxyureataxanes (such as paclitaxel and doxetaxel) and/or anthracycline antibiotics, as well as combinations of agents such as, but not limited to, DA-EPOCH, CHOP, CVP or FOLFOX. In various embodiments, the dosages of such chemotherapeutic agents include, but is not limited to, about any of 10 mg/m², 20 mg/m², 30 mg/m², 40 mg/m², 50 mg/m², 60 mg/m², 75 mg/m², 80 mg/m², 90 mg/m², 100 mg/m², 120 mg/m², 150 mg/m², 175 mg/m², 200 mg/m², 210 mg/m², 220 mg/m², 230 mg/m², 240 mg/m², 250 mg/m², 260 mg/m², and 300 mg/m².

### Immunotherapy

Numerous cancer immunotherapy strategies have been the focus of extensive research and clinical evaluation including, but not limited to, treatment using depleting antibodies to specific tumor antigens (see, e.g., reviews by Blattman and Greenberg, Science, 305:200, 2004; Adams and Weiner, Nat Biotech, 23:1147, 2005; Vogal et al. J Clin Oncology, 20:719, 2002; Colombat et al., Blood, 97:101, 2001); treatment using antibody-drug conjugates (see, e.g., Ducry, Laurent (Ed.) Antibody Drug Conjugates. In: Methods in Molecular Biology. Book 1045. New York (N.Y.), Humana Press, 2013; Nature Reviews Drug Discovery 12, 259-260, April 2013); treatment using agonistic, antagonistic, or blocking antibodies to co-stimulatory or co-inhibitory molecules (immune checkpoints) such as CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), OX-40, CD137, GITR, LAGS, TIM-3, and VISTA (see, e.g., Sharon et al., Chin J Cancer, 33(9): 434-444, September 2014; Hodi et al., N Engl J Med, 2010; Topalian et al., N Engl J Med, 366:2443-54, 2012); treatment using bispecific T cell engaging antibodies (BiTE®) such as blinatumomab (see, e.g., U.S. Pat. No. 9,260,522; US Patent Application No. 20140302037); treatment involving administration of biological response modifiers such as IL-2, IL-12, IL-15, IL-21, GM-CSF, IFN-α, IFN-β, and IFN-γ (see, e.g., Sutlu T et al., Journ of Internal Medicine, 266(2):154-181, 2009; Joshi S PNAS USA, 106(29):12097-12102, 2009; Li Y et al., Journal of Translational Medicine, 7:11, 2009); treatment using therapeutic vaccines such as sipuleucel-T (see, e.g., Kantoff P W New England Journal of Medicine, 363(5):411-422, 2010; Schlom J., Journal of the National Cancer Institutes, 104(8):599-613, 2012); treatment using dendritic cell vaccines, or tumor antigen peptide vaccines; treatment using chimeric antigen receptor (CAR)-T cells (see, e.g., Rosenberg S A Nature Reviews Cancer, 8(4):299-308, 2008; Porter D L et al, New England Journal of Medicine, 365(8):725-733, 2011; Grupp S A et al., New England Journal of Medicine, 368(16):1509-151, 2013; U.S. Pat. No. 9,102,761; U.S. Pat. No. 9,101,584); treatment using CAR-NK cells (see, e.g., Glienke et al., Front Pharmacol, 6(21):1-7, February 2015); treatment using tumor infiltrating lymphocytes (TILs)(see e.g., Wu et al, Cancer J., 18(2): 160-175, 2012); treatment using adoptively transferred anti-tumor T cells (ex vivo expanded and/or TCR transgenic)(see e.g., Wrzesinski et al., J Immunother, 33(1): 1-7, 2010); treatment using TALL-104 cells; and treatment using immunostimulatory agents such as Toll-like receptor (TLR) agonists CpG and imiquimod (see, e.g., Krieg, Oncogene, 27:161-167, 2008; Lu, Front Immunol, 5(83):1-4, March 2014).

### Combination Therapy Methods of Use

In another aspect, the present invention relates to combination therapies designed to treat a proliferative disease (such as cancer) in an individual, comprising administering to the individual: a) a therapeutically effective amount of a TAA Ab-IFN fusion molecule, and b) immunotherapy, wherein the combination therapy provides increased effector cell killing of tumor cells, i.e., a synergy exists between the TAA Ab-IFN fusion molecule and the immunotherapy when co-administered.

In various embodiments, the proliferative disease is a cancer selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

In various embodiments, there is provided a combination therapy method of treating a proliferative disease in an individual, comprising administering to the individual a) an effective amount of an anti-TAA-IFN-α fusion molecule; and b) immunotherapy; wherein the combination therapy provides increased effector cell killing. In various embodiments, the immunotherapy is treatment using agonistic, antagonistic, or blocking antibodies to co-stimulatory or co-inhibitory molecules. In various embodiments, the immunotherapy is treatment using chimeric antigen receptor (CAR)-T cells. In various embodiments, the immunotherapy is treatment using CAR-NK cells. In various embodiments, the immunotherapy is treatment using bispecific T cell engaging antibodies (BiTE®). In various embodiments, the anti-TAA-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg. In various embodiments, the cancer expresses the TAA of the anti-TAA Ab-IFN-α fusion molecule of the present invention. In various embodiments, the cancer is a non-TAA expressing cancer in the tumor microenvironment of a TAA expressing cancer. In various embodiments, the immunotherapy will target a TAA that is different than the TAA targeted by the TAA Ab-IFN fusion molecule.

In various embodiments, there is provided a combination therapy method of treating a cancer in an individual, comprising administering to the individual a) an effective amount of a pharmaceutical composition comprising an anti-HER2/neu-IFN-α fusion molecule; and b) immunotherapy using agonistic, antagonistic, or blocking antibodies to co-stimulatory or co-inhibitory molecules (immune checkpoints). In various embodiments, the cancer is selected from the group consisting of breast cancer, ovarian cancer and non-small cell lung cancer (NSCLC), and the anti-HER2/neu-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses HER2/neu. In various embodiments, the cancer is a non-HER2/neu expressing cancer in the tumor microenvironment of a HER2/neu expressing cancer.

In various embodiments, there is provided a combination therapy method of treating a cancer in an individual, comprising administering to the individual a) an effective amount of a pharmaceutical composition comprising an anti-HER2/neu-IFN-α fusion molecule; and b) immunotherapy using chimeric antigen receptor (CAR)-T cells. In various embodiments, the cancer is selected from the group consisting of breast cancer, ovarian cancer and non-small cell lung cancer (NSCLC), and the anti-HER2/neu-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses HER2/neu. In various embodiments, the cancer is a non-HER2/neu expressing cancer in the tumor microenvironment of a HER2/neu expressing cancer. In various embodiments, the CAR-T immunotherapy will target a TAA that is different than HER2/neu.

In various embodiments, there is provided a combination therapy method of treating a cancer in an individual, comprising administering to the individual a) an effective amount of a pharmaceutical composition comprising an anti-HER2/neu-IFN-α fusion molecule; and b) immunotherapy using treatment using CAR-NK cells. In various embodiments, the cancer is selected from the group consisting of breast cancer, ovarian cancer and non-small cell lung cancer (NSCLC), and the anti-HER2/neu-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses HER2/neu. In various embodiments, the cancer is a non-HER2/neu expressing cancer in the tumor microenvironment of a HER2/neu expressing cancer. In various embodiments, the CAR-NK immunotherapy will target a TAA that is different than HER2/neu.

In various embodiments, there is provided a combination therapy method of treating a cancer in an individual, comprising administering to the individual a) an effective amount of a pharmaceutical composition comprising an anti-HER2/neu-IFN-α fusion molecule; and b) immunotherapy using bispecific T cell engaging antibodies (BiTE®). In various embodiments, the cancer is selected from the group consisting of breast cancer, ovarian cancer and non-small cell lung cancer (NSCLC), and the anti-HER2/neu-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses HER2/neu. In various embodiments, the cancer is a non-HER2/neu expressing cancer in the tumor microenvironment of a HER2/neu expressing cancer. In various embodiments, the BiTE® immunotherapy will target a TAA that is different than HER2/neu.

In various embodiments, there is provided a combination therapy method of treating a cancer in an individual, comprising administering to the individual a) an effective amount of a pharmaceutical composition comprising an anti-CD20-IFN-α fusion molecule; and b) immunotherapy using agonistic, antagonistic, or blocking antibodies to co-stimulatory or co-inhibitory molecules (immune checkpoints). In various embodiments, the cancer is selected from the group consisting of B-cell Non-Hodgkin's lymphoma (NHL) and B-cell chronic lymphocytic leukemia (CLL), and the anti-CD20-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD20. In various embodiments, the cancer is a non-CD20 expressing cancer in the tumor microenvironment of a CD20 expressing cancer.

In various embodiments, there is provided a combination therapy method of treating a cancer in an individual, comprising administering to the individual a) an effective amount of a pharmaceutical composition comprising an anti-CD20-IFN-α fusion molecule; and b) immunotherapy using chimeric antigen receptor (CAR)-T cells. In various embodiments, the cancer is selected from the group consisting of B-cell Non-Hodgkin's lymphoma (NHL) and B-cell chronic lymphocytic leukemia (CLL), and the anti-CD20-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD20. In various embodiments, the cancer is a non-CD20 expressing cancer in the tumor microenvironment of a CD20 expressing cancer. In various embodiments, the CAR-T immunotherapy will target a TAA that is different than CD20.

In various embodiments, there is provided a combination therapy method of treating a cancer in an individual, comprising administering to the individual a) an effective amount of a pharmaceutical composition comprising an anti-CD20-IFN-α fusion molecule; and b) immunotherapy using treatment using CAR-NK cells. In various embodiments, the cancer is selected from the group consisting of B-cell Non-Hodgkin's lymphoma (NHL) and B-cell chronic lymphocytic leukemia (CLL), and the anti-CD20-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD20. In various embodiments, the cancer is a non-CD20 expressing cancer in the tumor microenvironment of a CD20 expressing cancer. In various embodiments, the CAR-NK immunotherapy will target a TAA that is different than CD20.

In various embodiments, there is provided a combination therapy method of treating a cancer in an individual, comprising administering to the individual a) an effective amount of a pharmaceutical composition comprising an anti-CD20-IFN-α fusion molecule; and b) immunotherapy using bispecific T cell engaging antibodies (BiTE®). In various embodiments, the cancer is selected from the group consisting of B-cell Non-Hodgkin's lymphoma (NHL) and B-cell chronic lymphocytic leukemia (CLL), and the anti-CD20-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD20. In various embodiments, the cancer is a non-CD20 expressing cancer in the tumor microenvironment of a CD20 expressing cancer. In various embodiments, the BiTE® immunotherapy will target a TAA that is different than CD20.

In various embodiments, there is provided a combination therapy method of treating a cancer in an individual, comprising administering to the individual a) an effective amount of a pharmaceutical composition comprising an anti-CD138-IFN-α fusion molecule; and b) immunotherapy using agonistic, antagonistic, or blocking antibodies to co-stimulatory or co-inhibitory molecules (immune checkpoints). In various embodiments, the cancer is selected from the group consisting of multiple myeloma, breast cancer, and bladder cancer, and the anti-CD138-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD138. In various embodiments, the cancer is a non-CD138 expressing cancer in the tumor microenvironment of a CD138 expressing cancer.

In various embodiments, there is provided a combination therapy method of treating a cancer in an individual, comprising administering to the individual a) an effective amount of a pharmaceutical composition comprising an anti-CD138-IFN-α fusion molecule; and b) immunotherapy using chimeric antigen receptor (CAR)-T cells. In various embodiments, the cancer is selected from the group consisting of multiple myeloma, breast cancer, and bladder cancer, and the anti-CD138-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD138. In various embodiments, the cancer is a non-CD138 expressing cancer in the tumor microenvironment of a CD138 expressing cancer. In various embodiments, the CAR-T immunotherapy will target a TAA that is different than CD138.

In various embodiments, there is provided a combination therapy method of treating a cancer in an individual, comprising administering to the individual a) an effective amount of a pharmaceutical composition comprising an anti-CD138-IFN-α fusion molecule; and b) immunotherapy using treatment using CAR-NK cells. In various embodiments, the cancer is selected from the group consisting of multiple myeloma, breast cancer, and bladder cancer, and the anti-CD138-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD138. In various embodiments, the cancer is a non-CD138 expressing cancer in the tumor microenvironment of a CD138 expressing cancer. In various embodiments, the CAR-NK immunotherapy will target a TAA that is different than CD138.

In various embodiments, there is provided a combination therapy method of treating a cancer in an individual, comprising administering to the individual a) an effective amount of a pharmaceutical composition comprising an anti-CD138-IFN-α fusion molecule; and b) immunotherapy using bispecific T cell engaging antibodies (BiTE®). In various embodiments, the cancer is selected from the group consisting of multiple myeloma, breast cancer, and bladder cancer, and the anti-CD138-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD138. In various embodiments, the cancer is a non-CD138 expressing cancer in the tumor microenvironment of a CD138 expressing cancer. In various embodiments, the BiTE® immunotherapy will target a TAA that is different than CD138.

In various embodiments, there is provided a combination therapy method of treating a cancer in an individual, comprising administering to the individual a) an effective amount of a pharmaceutical composition comprising an anti-GRP94-IFN-α fusion molecule; and b) immunotherapy using agonistic, antagonistic, or blocking antibodies to co-stimulatory or co-inhibitory molecules (immune checkpoints). In various embodiments, the cancer is selected from the group consisting of NSCLC, acute myeloid leukemia (AML), multiple myeloma, melanoma, and pancreatic cancer, and the anti-GRP94-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses GRP94. In various embodiments, the cancer is a non-GRP94 expressing cancer in the tumor microenvironment of a GRP94 expressing cancer.

In various embodiments, there is provided a combination therapy method of treating a cancer in an individual, comprising administering to the individual a) an effective amount of a pharmaceutical composition comprising an anti-GRP94-IFN-α fusion molecule; and b) immunotherapy using chimeric antigen receptor (CAR)-T cells. In various embodiments, the cancer is selected from the group consisting of NSCLC, acute myeloid leukemia (AML), multiple myeloma, melanoma, and pancreatic cancer, and the anti-GRP94-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses GRP94. In various embodiments, the cancer is a non-GRP94 expressing cancer in the tumor microenvironment of a GRP94 expressing cancer. In various embodiments, the CAR-T immunotherapy will target a TAA that is different than GRP94.

In various embodiments, there is provided a combination therapy method of treating a cancer in an individual, comprising administering to the individual a) an effective amount of a pharmaceutical composition comprising an anti-GRP94-IFN-α fusion molecule; and b) immunotherapy using treatment using CAR-NK cells. In various embodiments, the cancer is selected from the group consisting of NSCLC, acute myeloid leukemia (AML), multiple myeloma, melanoma, and pancreatic cancer, and the anti-GRP94-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses GRP94. In various embodiments, the cancer is a non-GRP94 expressing cancer in the tumor microenvironment of a GRP94 expressing cancer. In various embodiments, the CAR-NK immunotherapy will target a TAA that is different than GRP94.

In various embodiments, there is provided a combination therapy method of treating a cancer in an individual, comprising administering to the individual a) an effective amount of a pharmaceutical composition comprising an anti-GRP94-IFN-α fusion molecule; and b) immunotherapy using bispecific T cell engaging antibodies (BiTE®). In various embodiments, the cancer is selected from the group consisting of NSCLC, acute myeloid leukemia (AML), multiple myeloma, melanoma, and pancreatic cancer, and the anti-GRP94-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses GRP94. In various embodiments, the cancer is a non-GRP94 expressing cancer in the tumor microenvironment of a GRP94 expressing cancer. In various embodiments, the BiTE® immunotherapy will target a TAA that is different than GRP94.

In various embodiments, there is provided a combination therapy method of treating a cancer in an individual, comprising administering to the individual a) an effective amount of a pharmaceutical composition comprising an anti-CD33-IFN-α fusion molecule; and b) immunotherapy using agonistic, antagonistic, or blocking antibodies to co-stimulatory or co-inhibitory molecules (immune checkpoints). In various embodiments, the cancer is selected from the group consisting of AML, chronic myeloid leukemia (CML) and multiple myeloma, and the anti-CD33-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD33. In various embodiments, the cancer is a non-CD33 expressing cancer in the tumor microenvironment of a CD33 expressing cancer.

In various embodiments, there is provided a combination therapy method of treating a cancer in an individual, comprising administering to the individual a) an effective amount of a pharmaceutical composition comprising an anti-CD33-IFN-α fusion molecule; and b) immunotherapy using chimeric antigen receptor (CAR)-T cells. In various embodiments, the cancer is selected from the group consisting of AML, chronic myeloid leukemia (CML) and multiple myeloma, and the anti-CD33-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD33. In various embodiments, the cancer is a non-CD33 expressing cancer in the tumor microenvironment of a CD33 expressing cancer. In various embodiments, the CAR-T immunotherapy will target a TAA that is different than CD33.

In various embodiments, there is provided a combination therapy method of treating a cancer in an individual, comprising administering to the individual a) an effective amount of a pharmaceutical composition comprising an anti-CD33-IFN-α fusion molecule; and b) immunotherapy using treatment using CAR-NK cells. In various embodiments, the cancer is selected from the group consisting of AML, chronic myeloid leukemia (CML) and multiple myeloma, and the anti-CD33-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD33. In various embodiments, the cancer is a non-CD33 expressing cancer in the tumor microenvironment of a CD33 expressing cancer. In various embodiments, the CAR-NK immunotherapy will target a TAA that is different than CD33.

In various embodiments, there is provided a combination therapy method of treating a cancer in an individual, comprising administering to the individual a) an effective amount of a pharmaceutical composition comprising an anti-CD33-IFN-α fusion molecule; and b) immunotherapy using bispecific T cell engaging antibodies (BiTE®). In various embodiments, the cancer is selected from the group consisting of AML, chronic myeloid leukemia (CML) and multiple myeloma, and the anti-CD33-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD33. In various embodiments, the cancer is a non-CD33 expressing cancer in the tumor microenvironment of a CD33 expressing cancer. In various embodiments, the BiTE® immunotherapy will target a TAA that is different than CD33.

In various embodiments, there is provided a combination therapy method of treating a cancer in an individual, comprising administering to the individual a) an effective amount of a pharmaceutical composition comprising an anti-CD70-IFN-α fusion molecule; and b) immunotherapy using agonistic, antagonistic, or blocking antibodies to co-stimulatory or co-inhibitory molecules (immune checkpoints); wherein the combination therapy provides increased effector cell killing. In various embodiments, the cancer is selected from the group consisting of renal cell carcinoma (RCC), Waldenstrom macroglobulinemia, multiple myeloma, and NHL, and the anti-CD70-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD70. In various embodiments, the cancer is a non-CD70 expressing cancer in the tumor microenvironment of a CD70 expressing cancer.

In various embodiments, there is provided a combination therapy method of treating a cancer in an individual, comprising administering to the individual a) an effective amount of a pharmaceutical composition comprising an anti-CD70-IFN-α fusion molecule; and b) immunotherapy using chimeric antigen receptor (CAR)-T cells. In various embodiments, the cancer is selected from the group consisting of renal cell carcinoma (RCC), Waldenstrom macroglobulinemia, multiple myeloma, and NHL, and the anti-CD70-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD70. In various embodiments, the cancer is a non-CD70 expressing cancer in the tumor microenvironment of a CD70 expressing cancer. In various embodiments, the CAR-T immunotherapy will target a TAA that is different than CD70.

In various embodiments, there is provided a combination therapy method of treating a cancer in an individual, comprising administering to the individual a) an effective amount of a pharmaceutical composition comprising an anti-CD70-IFN-α fusion molecule; and b) immunotherapy using treatment using CAR-NK cells. In various embodiments, the cancer is selected from the group consisting of renal cell carcinoma (RCC), Waldenstrom macroglobulinemia, multiple myeloma, and NHL, and the anti-CD70-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD70. In various embodiments, the cancer is a non-CD70 expressing cancer in the tumor microenvironment of a CD70 expressing cancer. In various embodiments, the CAR-NK immunotherapy will target a TAA that is different than CD70.

In various embodiments, there is provided a combination therapy method of treating a cancer in an individual, comprising administering to the individual a) an effective amount of a pharmaceutical composition comprising an anti-CD70-IFN-α fusion molecule; and b) immunotherapy using bispecific T cell engaging antibodies (BiTE®). In various embodiments, the cancer is selected from the group consisting of renal cell carcinoma (RCC), Waldenstrom macroglobulinemia, multiple myeloma, and NHL, and the anti-CD70-IFN-α fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, and about 0.1 to about 0.3 mg/kg. In various embodiments, the cancer expresses CD70. In various embodiments, the cancer is a non-CD70 expressing cancer in the tumor microenvironment of a CD70 expressing cancer. In various embodiments, the BiTE® immunotherapy will target a TAA that CD70.

In various embodiments, the combination therapy methods comprise administering the TAA Ab-IFN fusion molecule and immunotherapy simultaneously, either in the same pharmaceutical composition or in separate pharmaceutical compositions. Alternatively, the TAA Ab-IFN fusion molecule and immunotherapy are administered sequentially, i.e., the TAA Ab-IFN fusion molecule is administered either prior to or after the immunotherapy.

In various embodiments, the administration of the TAA Ab-IFN fusion molecule and immunotherapy are concurrent, i.e., the administration period of the TAA Ab-IFN fusion molecule and immunotherapy overlap with each other.

In various embodiments, the administration of the TAA Ab-IFN fusion molecule and immunotherapy are non-concurrent. For example, in various embodiments, the TAA Ab-IFN fusion molecule is administered prior to the administration of immunotherapy. In various embodiments, the TAA Ab-IFN fusion molecule is administered at a time which is selected from the group consisting of: about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, and about 1 week prior to administration of immunotherapy.

In various embodiments, the immunotherapy is administered prior to the administration of TAA Ab-IFN fusion molecule. In various embodiments, the immunotherapy is administered at a time which is selected from the group consisting of: about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, and about 1 week prior to administration of TAA Ab-IFN fusion molecule.

In various embodiments, the administration of the TAA Ab-IFN fusion molecule is terminated before the immunotherapy is administered. In some embodiments, the administration of immunotherapy is terminated before the TAA Ab-IFN fusion molecule is administered.

These various combination therapies may provide a “synergistic effect”, i.e., the effect achieved when the active ingredients used together is greater than the sum of the effects that results from using the compounds separately.

Nucleic acid Molecules and Fusion Molecule Expression

The present application further provides nucleic acid molecules comprising nucleotide sequences encoding the recombinant, genetically engineered fusion molecules described herein. Because of the degeneracy of the genetic code, a variety of nucleic acid sequences encode each fusion molecule amino acid sequence. The application further provides nucleic acid molecules that hybridize under stringent or lower stringency hybridization conditions, e.g., as defined herein, to nucleic acid molecules that encode a fusion molecule. Stringent hybridization conditions include, but are not limited to, hybridization to filter-bound DNA in 6×SSC at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3).

The nucleic acid molecules may be obtained, and the nucleotide sequence of the nucleic acid molecules determined by, any method known in the art. For example, if the nucleotide sequence of the fusion molecule is known, a nucleic acid molecule encoding the fusion molecule may be assembled from chemically synthesized oligonucleotides (e.g., as described in Kutmeier et al., BioTechniques 17:242, 1994), which, briefly, involves the synthesis of overlapping oligonucleotides containing portions of the sequence encoding the antibody, annealing and ligating of those oligonucleotides, and then amplification of the ligated oligonucleotides by PCR. In one embodiment, the codons that are used comprise those that are typical for human or mouse (see, e.g., Nakamura, Y., Nucleic Acids Res. 28: 292, 2000).

A nucleic acid molecule encoding a fusion molecule may also be generated from nucleic acid from a suitable source. For example, if a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably polyA+RNA, isolated from, any tissue or cells expressing the antibody, such as hybridoma cells selected to express an antibody) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

In one embodiment of the present disclosure, nucleic acid sequences encoding the appropriate antibody framework are optionally cloned and ligated into appropriate vectors (e.g., expression vectors for, e.g., prokaryotic or eukaryotic organisms). Additionally, nucleic acid sequences encoding the appropriate interferon molecule are optionally cloned into the same vector in the appropriate orientation and location so that expression from the vector produces an antibody-interferon molecule fusion molecule. Some optional embodiments also require post-expression modification, e.g., assembly of antibody subunits, etc. The techniques and art for the above (and similar) manipulations are well known to those skilled in the art. Pertinent instructions are found in, e.g., Sambrook et al., Molecular Cloning—A Laboratory Manual (2nd Ed.), Vols. 1-3, Cold Spring Harbor Laboratory, Cold Spring Harbor, N.Y., 1989 and Current Protocols in Molecular Biology, F. M. Ausubel et al., eds., Current Protocols, a joint venture between Greene Publishing Associates, Inc. and John Wiley & Sons, Inc. (supplemented through 1999).

The present disclosure is also directed to host cells that express the fusion molecules of the disclosure. Host cells suitable for replicating and for supporting recombinant expression of fusion protein are well known in the art. Such cells may be transfected or transduced as appropriate with the particular expression vector and large quantities of vector containing cells can be grown for seeding large scale fermenters to obtain sufficient quantities of the protein for clinical applications. Such cells may include prokaryotic microorganisms, such as E. coli; various eukaryotic cells, such as Chinese hamster ovary cells (CHO), NSO, 293; HEK Yeast; insect cells; hybridomas; human cell lines; and transgenic animals and transgenic plants, and the like. Standard technologies are known in the art to express foreign genes in these systems. The recombinant protein gene is typically operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and preferably an enhancer derived from immunoglobulin genes, SV40, cytomegalovirus, etc., and a polyadenylation sequence, and may include splice donor and acceptor sequences.

To express an antibody-IFN fusion molecule recombinantly, a host cell is transformed, transduced, infected or the like with one or more recombinant expression vectors carrying DNA fragments encoding the immunoglobulin light and/or heavy chains of the antibody and attached interferon such that the light and/or heavy chains are expressed in the host cell. The heavy chain and the light chain may be expressed independently from different promoters to which they are operably-linked in one vector or, alternatively, the heavy chain and the light chain may be expressed independently from different promoters to which they are operably-linked in two vectors one expressing the heavy chain and one expressing the light chain. Optionally, the heavy chain and light chain may be expressed in different host cells.

Additionally, the recombinant expression vector can encode a signal peptide that facilitates secretion of the antibody light and/or heavy chain from a host cell. The antibody light and/or heavy chain gene can be cloned into the vector such that the signal peptide is operably-linked in-frame to the amino terminus of the antibody chain gene. The signal peptide can be an immunoglobulin signal peptide or a heterologous signal peptide. Preferably, the recombinant antibodies are secreted into the medium in which the host cells are cultured, from which the antibodies can be recovered or purified.

An isolated DNA encoding a HCVR can be converted to a full-length heavy chain gene by operably-linking the HCVR-encoding DNA to another DNA molecule encoding heavy chain constant regions. The sequences of human, as well as other mammalian, heavy chain constant region genes are known in the art. DNA fragments encompassing these regions can be obtained e.g., by standard PCR amplification. The heavy chain constant region can be of any type, (e.g., IgG, IgA, IgE, IgM or IgD), class (e.g., IgG₁, IgG₂, IgG₃ and IgG₄) or subclass constant region and any allotypic variant thereof as described in Kabat (supra).

An isolated DNA encoding a LCVR region may be converted to a full-length light chain gene (as well as to a Fab light chain gene) by operably linking the LCVR-encoding DNA to another DNA molecule encoding a light chain constant region. The sequences of human, as well as other mammalian, light chain constant region genes are known in the art. DNA fragments encompassing these regions can be obtained by standard PCR amplification. The light chain constant region can be a kappa or lambda constant region.

Additionally, the recombinant expression vectors of the disclosure may carry additional sequences, such as sequences that regulate replication of the vector in host cells (e.g., origins of replication) and one or more selectable marker genes. The selectable marker gene facilitates selection of host cells into which the vector has been introduced. For example, typically the selectable marker gene confers resistance to drugs, such as G418, hygromycin, or methotrexate, on a host cell into which the vector has been introduced. Preferred selectable marker genes include the dihydrofolate reductase (dhfr) gene (for use in dhfr-minus host cells with methotrexate selection/amplification), the neo gene (for G418 selection), and glutamine synthetase (GS) in a GS-negative cell line (such as NSO) for selection/amplification.

For expression of the light and/or heavy chains with attached interferon, the expression vector(s) encoding the heavy and/or light chains is introduced into a host cell by standard techniques e.g. electroporation, calcium phosphate precipitation, DEAE-dextran transfection, transduction, infection and the like. Although it is theoretically possible to express the antibodies of the disclosure in either prokaryotic or eukaryotic host cells, eukaryotic cells and most specifically mammalian host cells, are more typical because such cells are more likely to assemble and secrete a properly folded and immunologically active antibody. Mammalian host cells for expressing the recombinant antibodies of the disclosure include Chinese Hamster Ovary (CHO cells) [including dhfr minus CHO cells, as described in Urlaub and Chasin, Proc. Natl. Acad. Sci. USA 77:4216-20, 1980, used with a DHFR selectable marker, e.g. as described in Kaufman and Sharp, J. Mol. Biol. 159:601-21, 1982], NSO myeloma cells, COS cells, and SP2/0 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown under appropriate conditions known in the art. Antibodies can be recovered from the host cell and/or the culture medium using standard purification methods.

Once expressed, the intact antibodies, individual light and heavy chains, or other immunoglobulin forms of the present disclosure can be purified according to standard procedures of the art, including ammonium sulfate precipitation, ion exchange, affinity (e.g., Protein A), reverse phase, hydrophobic interaction column chromatography, hydroxyapatite chromatography, gel electrophoresis, and the like. Standard procedures for purification of therapeutic antibodies are described, for example, by Feng L1, Joe X. Zhou, Xiaoming Yang, Tim Tressel, and Brian Lee in an article entitled “Current Therapeutic Antibody Production and Process Optimization” (BioProcessing Journal, September/October 2005), for example. Additionally, standard techniques for removing viruses from recombinantly expressed antibody preparations are also known in the art (see, for example, Gerd Kern and Mani Krishnan, “Viral Removal by Filtration: Points to Consider” (Biopharm International, October 2006)). The effectiveness of filtration to remove viruses from preparations of therapeutic antibodies is known to be at least in part dependent on the concentration of protein and/or the antibody in the solution to be filtered. The purification process for antibodies of the present disclosure may include a step of filtering to remove viruses from the mainstream of one or more chromatography operations. Preferably, prior to filtering through a pharmaceutical grade nanofilter to remove viruses, a chromatography mainstream containing an antibody of the present disclosure is diluted or concentrated to give total protein and/or total antibody concentration of about 1 g/L to about 3 g/L. Even more preferably, the nanofilter is a DV20 nanofilter (e.g., Pall Corporation; East Hills, N.Y.). Substantially pure immunoglobulins of at least about 90%, about 92%, about 94% or about 96% homogeneity are preferred, and about 98 to about 99% or more homogeneity most preferred, for pharmaceutical uses. Once purified, partially or to homogeneity as desired, the sterile antibodies may then be used therapeutically, as directed herein.

In view of the aforementioned discussion, the present disclosure is further directed to a fusion molecule obtainable by a process comprising the steps of culturing a host cell including, but not limited to a mammalian, plant, bacterial, transgenic animal, or transgenic plant cell which has been transformed by a nucleic acid molecule or a vector comprising nucleic acid molecules encoding antibodies of the disclosure so that the nucleic acid is expressed and, optionally, recovering the antibody from the host cell culture medium.

### Kits

In certain embodiments, this disclosure provides for kits for the treatment of cancer and/or in an adjunct therapy. Kits typically comprise a container containing a TAA Ab-IFN fusion molecule of the present disclosure. The TAA Ab-IFN fusion molecule can be present in a pharmacologically acceptable excipient. The kits may optionally include an immunotherapy cancer agent.

In addition, the kits can optionally include instructional materials disclosing means of use of the TAA Ab-IFN fusion molecule and/or immunotherapy to treat a cancer. The instructional materials may also, optionally, teach preferred dosages, counter-indications, and the like.

The kits can also include additional components to facilitate the particular application for which the kit is designed. Thus, for example, and additionally comprise means for disinfecting a wound, for reducing pain, for attachment of a dressing, and the like.

While the instructional materials typically comprise written or printed materials they are not limited to such. Any medium capable of storing such instructions and communicating them to an end user is contemplated by this disclosure. Such media include, but are not limited to electronic storage media (e.g., magnetic discs, tapes, cartridges, chips), optical media (e.g., CD ROM), and the like. Such media may include addresses to internet sites that provide such instructional materials.

The following examples are provided to describe the invention in further detail.

### Example 1

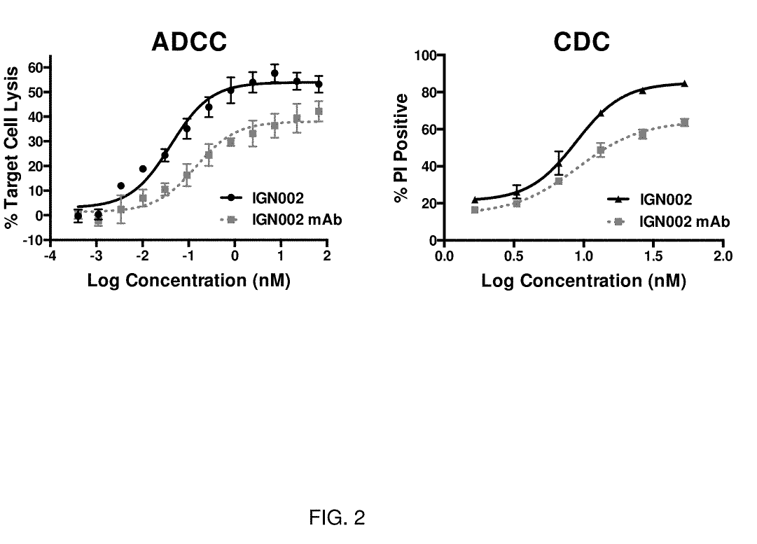

The present inventors previously reported that a recombinant anti-CD20 Ab-wt IFN-α2b fusion molecule (hereinafter referred to as “IGN002”) prepared as described herein demonstrated superior anti-lymphoma activity against numerous cell lines in vitro and against established human xenograft tumors grown in mice (Xuan et al., Blood 115: 2864-71, 2010; Timmerman J et al, Blood 126(23): 2762, 2015). Specifically, in nonclinical studies, IGN002 selectively bound to CD20-positive cells and exhibited potent anti-proliferative activity in vitro against CD20-positive NHL cell lines (EC₅₀ values of 0.1-2.1 pM) relative to each of the fusion partners alone. IGN002 also demonstrated enhanced cytokine-dependent cytotoxicity (CDC) and antibody-dependent cell-mediated cytotoxicity (ADCC) activity against NHL cells, compared to rituximab, and exhibited potent pro-apoptotic activity against NHL cell lines (EC₅₀ values of 1.9 pM-2.7 nM)(see FIG. 2). Notably, antiviral activity was reduced by 270-fold for IGN002, compared to non-fused IFN-α, suggesting the potential for a higher therapeutic index for IGN002 due to attenuation of systemic adverse effects (AEs) compared to molar equivalent levels of non-fused IFNα.

Xenograft studies in immunodeficient mice using three different human NHL cell lines compared the anti-tumor activity of IGN002 to rituximab and a control mAb-IFN-α fusion molecule. Against all three NHL xenograft tumor lines, IGN002 demonstrated superior anti-tumor efficacy in vivo, as measured by median survival and overall survival. Against Raji Burkitt lymphoma tumors, IGN002 possessed equivalent efficacy when administered at a 25-fold lower molar dose than rituximab. Against Daudi Burkitt lymphoma tumors, 100% of animals treated with IGN002 experienced complete tumor regression and survived for the duration of the study, whereas only 12.5% treated with rituximab at equimolar dose levels survived (p≤0.0005). IGN002 treatment of OCI-Ly19 DLBCL xenograft tumor-bearing mice resulted in significantly longer survival than rituximab treatment (median survival of 96.5 versus 58 days, respectively, p<0.0001). The importance of CD20-targeted delivery of IFN-α was also demonstrated, as IGN002 treatment exhibited superior anti-tumor activity with a more pronounced delay in OCI-Ly19 tumor progression and a significantly longer median survival of 96.5 days, compared to 59 days for a non-targeted control mAb-IFN-α fusion molecule at the same dose (p<0.0001).

### Example 2

In this example, using the methods described herein, a TAA Ab-IFN fusion molecule comprising an anti-GRP94 antibody having the amino acid sequence set forth in SEQ ID NO: 12 and a light chain having the amino acid sequence set forth in SEQ ID NO: 13 was prepared as follows: an interferon molecule having the amino acid sequence set forth in SEQ ID NO: 1 was attached to the C-terminus of the anti-GRP94 Ab heavy chain using a linker having the amino acid sequence of SEQ ID NO: 18 (this fusion hereinafter referred to as “IGN004”).

The expression of GRP94 on various solid tumor and hematological cancer cell lines was assessed by flow cytometry. Cells were incubated with anti-GRP94 Ab at 2 μg per 10⁶ cells for 1 hour on ice. After incubation, cells were washed twice then bound mAb was detected with anti-human IgG(Fc)-FITC Ab. Samples were analyzed by flow cytometry using a Beckman Coulter Cytomics FC500 or Galios flow cytometer instrument and data analyzed using WinMDI software. Controls included anti-GRP94 Ab followed by an IgG2a,κ-FITC isotype control and anti-human IgG (Fc-specific)-FITC alone. The flow cytometry results are depicted in Table 6. Anti-GRP94 Ab bound to the majority of tumor cell lines investigated, including melanoma, NSCLC, AML, and MM.

TABLE 6

| | # Positive Cell |
|---|---|
| Tumor Type | Types |
| AML | 10/10 |
| LUNG | 6/10 |
| MELANOMA | 5/5 |
| BREAST | 3/5 |
| MULTIPLE MYELOMA | 3/4 |
| OVARIAN | 2/3 |
| COLORECTAL | 2/2 |

The expression of GRP94 on various primary human tumors and patient-derived human xenograft tumors (grown in immunodeficient mice) was evaluated using Immunohistochemistry (IHC). The IHC results are depicted in Table 7.

TABLE 7

| | # Positive |
|---|---|
| Primary Tumor | Tumors |
| LUNG | 10/10 |
| LUNG PDX | 50/54 |
| MELANOMA | 7/7 |
| PANCREATIC | 4/7 |
| PANCREATIC PDX | 40/43 |
| BREAST | 2/5 |
| CANCER | 2/5 |
| COLORECTAL | 1/5 |

Again, anti-GRP94 antibody bound to nearly 100% of the primary solid tumor samples tested by IHC.

### Example 3

In this example, the STAT1 phosphorylation and proliferation inhibition activities of IGN004 were compared to non-fused IFN-α2b in a non-targeted and a targeted setting, respectively.

For the non-targeted STAT1 phosphorylation experiment, Daudi NHL tumor cells (GRP94-negative) were incubated with the indicated concentration of IGN004 or IFN-α2b for 15 minutes, then cells were fixed, permeabilized and intracellularly stained with PE-labeled anti-STAT1 (pY701) or PE-labeled isotype control. After washing, samples were analyzed by flow cytometry. Dose response curves were generated by non-linear regression analysis using Prism software. For the targeted proliferation inhibition experiment, GRP94-positive NCI-H1299 NSCLC tumor cells (ATCC CRL-5803) were treated with the indicated concentration of IGN0004 or IFN-α2b for 96 hours at 37° C. in a 5% CO₂ atmosphere. After incubation, standard MTS assay (Promega Cell Titer96; Promega, Madison, Wis.) was performed to assess cellular proliferation. Dose response curves were generated by non-linear regression analysis using Prism software.

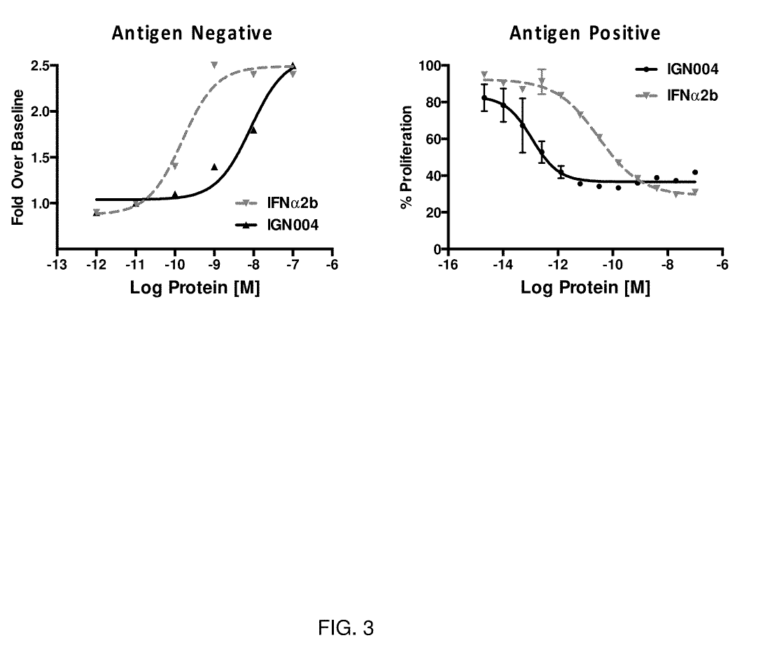

As depicted in FIG. 3, IGN004 relative IFN activity was reduced on antigen-negative cells (Daudi) and enhanced on antigen-positive cells (NCI-H1299). The STAT1-phosphorylation activity was attenuated by 54-fold, compared to non-fused IFN-α2b (EC₅₀(IFN-α2b)=0.154 nM and EC₅₀(IGN004)=8.39 nM), on Daudi cells that do not express the antibody target antigen. The proliferation inhibition activity was enhanced by 275-fold, compared to non-fused IFN-α2b (EC₅₀(IFN-α2b)=30.3 pM and EC₅₀(IGN004)=0.11 pM), on NCI-H1299 cells that express the antibody target antigen.

### Example 4

In this example, the in vivo anti-tumor activity of IGN004 was investigated in a xenograft model of human multiple myeloma where U266 tumors were grown in NOG (NOD/Shi-scid/IL-2Rγ^{null}) immunodeficient mice (Ito et al, Blood, 100(9): 3175-82, 2002).

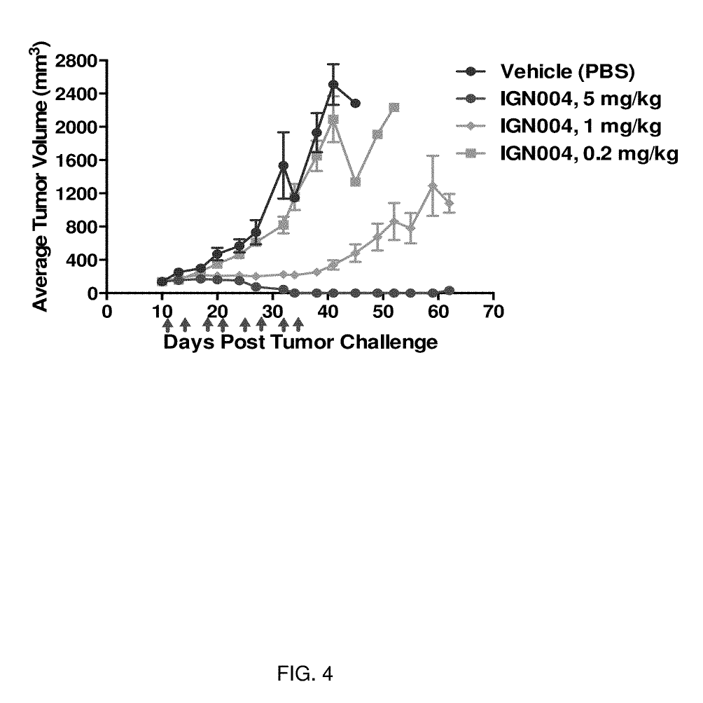

In this study, groups of 8 11-day established subcutaneous tumor-bearing animals (average tumor volume=138 mm³) were treated intravenously, twice per week, for 4 weeks with 5, 1 or 0.2 mg/kg IGN004. Vehicle (PBS) treatment served as a negative control. Animals treated with PBS had a median survival time of 39.5 days, and treatment with 0.2 mg/kg IGN004 did not significantly extend survival (see FIG. 4). However, treatment with 1 mg/kg IGN004 significantly improved survival, with median survival of 47 days (p=0.02 vs. PBS) and 73 days (p=0.0002 vs. PBS), respectively. Treatment of animals with 5 mg/kg IGN004 resulted in complete regression of established tumors in 100% of the mice. These data demonstrate that IGN004 can effectively treat human multiple myeloma xenograft tumors.

### Example 5

In this example, the in vivo anti-tumor activity of IGN004 was assessed against a panel of 15 different human NSCLC patient-derived xenograft (PDX) tumors grown in immunodeficient mice.

Groups of 5 BALB/c nude immunodeficient mice bearing established NSCLC PDX tumors with an average tumor volume of 150 mm³ were treated with either PBS or 2 mg/kg IGN004 intravenously twice per week for the duration of the experiment. Tumor size was measured bidirectionally using calipers twice weekly, and tumor volume calculated using the formula: V=0.5 a×b² where a and b are the long and short diameters of the tumor, respectively. Average tumor volume at each time point for each tumor model was plotted using Excel software (Microsoft) and efficacy of IGN004 was sorted into 4 categories: +++=tumor regression, ++=stable disease, +=slowing of tumor growth, and −=no response.

As depicted in Table 8, IGN004 demonstrated in vivo efficacy on 10/15 PDX tumors (66.7%), including tumor regression in 4 tumor models. There did not appear to be a correlation with known gene mutations nor NSCLC tumor type and response to treatment. These results show that TAA Ab-IFN fusion molecules like IGN004 can be highly effective against clinically-relevant NSCLC PDX tumors, even in the absence of immune cells which may potentially play a role in the mechanism of action of TAA Ab-IFN fusion molecules in human cancer patients.

TABLE 8

| Model # | Type | Mutations | Efficacy |
|---|---|---|---|
| 1 | Adenosquamous | p53 | +++ |
| 2 | Adenocarcinoma | ND | +++ |
| 3 | Large-cell | ND | +++ |
| | carcinoma | | |
| 4 | Squamous cell | p53, ALK | +++ |
| | carcinoma | | |
| 5 | Squamous cell | AKT, p53 | ++ |
| | carcinoma | | |
| 6 | Squamous cell | EGFR | ++ |
| | carcinoma | | |
| 7 | Adenocarcinoma | EGFR, p53 | + |
| 8 | Adenocarcinoma | EGFR | + |
| 9 | Adenosquamous | c-Met | + |
| 10 | Adenocarcinoma | p53 | + |
| 11 | Squamous cell | ND | − |
| | carcinoma | | |
| 12 | Adenocarcinoma | ND | − |
| 13 | Squamous cell | p53 | − |
| | carcinoma | | |
| 14 | Squamous cell | p53, PTEN | − |
| | carcinoma | | |
| 15 | Squamous cell | p53 | − |
| | carcinoma | | |

### Example 6

In this example, the tumor cell killing activity of the human CD8+ NKT cell-like TALL-104 effector cell line (ATCC CRL-11386) was assessed in the presence or absence of IGN004 using the A549 human NSCLC tumor cell line (ATCC CCL-185).

TALL-104 cells growing in 300 U/mL IL-2 were washed twice to remove IL-2 and placed back into culture overnight. A549 tumor cells were plated in 24-well plates and incubated overnight at 37° C. in a 5% CO₂ atmosphere. The next day, cells were incubated with 3 nM IGN004 for 4 hours then wells were washed to remove unbound protein. After overnight incubation in the absence of IL-2, TALL-104 effector cells were then added to the wells containing A549 tumor cells to achieve an effector:target ratio (E:T) of 5:1. Co-cultures were incubated for 24 hours at 37° C. in a 5% CO₂ atmosphere then viability of the tumor cells was assessed by standard MTS assay after washing away the non-adherent effector cells. Wells were washed twice and then 0.5 mL of 4:1 mix RPMI+10% FBS and Promega Cell Titer96 was added and incubated for 1 hour at 37° C. Media was transferred to a 96 well plate and the plate was read at 490 nm using a spectrophotometer. Data was plotted in GraphPad Prism taking untreated tumor cells as 100% cell control and the mix of media and Cell Titer incubated for 1 hour at 37° C. as 0% cell control. Controls included A549 tumor cells alone, A549 tumor cells+IGN004 (no effectors), and A549 tumor cells+TALL-104 effector cells (no IGN004). Plates were set up with quadruplicate samples.

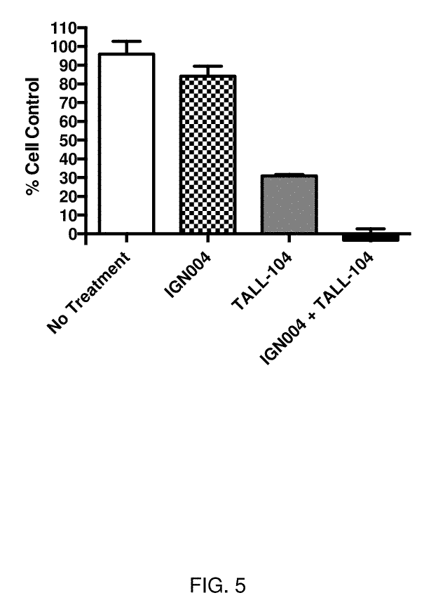

As depicted in FIG. 5, IGN004 treatment caused a small decrease in the viability of the A549 tumor cells (15.82%). TALL-104 effector cells demonstrated robust killing in the absence of IGN004 (69.2%). However, the combination of IGN004 and TALL-104 cells lead to complete eradication of A549 tumor cells (100% killing). This effect was stronger than the combination of either agent alone (85.02% vs. 100%), leading to the conclusion that IGN004 and TALL-104 can have a synergistic effect upon A549 tumor cells leading to much more robust tumor cell killing.

### Example 7

In this example, the tumor cell killing activity of TALL-104 effector cells was assessed in the presence or absence of IGN004 at two different E:T ratios using a different human NSCLC tumor cell line (NCI-H1975; ATCC CRL-5908).

TALL-104 cells growing in 300 U/mL IL-2 were washed twice to remove IL-2 and placed back into culture overnight. NCI-H1975 tumor cells were plated in 24-well plates and incubated overnight at 37° C. in a 5% CO₂ atmosphere. The next day, cells were incubated with 50 pM IGN004 for 4 hours then wells were washed to remove unbound protein. After overnight incubation in the absence of IL-2, TALL-104 effector cells were then added to the wells containing tumor cells to achieve an E:T ratio of 5:1 or 3.3:1. Co-cultures were incubated for 48 hours at 37° C. in a 5% CO₂ atmosphere then viability of the tumor cells was assessed as described previously in Example 1. Controls included NCI-H1975 tumor cells alone, NCI-H1975 tumor cells+IGN004 (no effectors), and NCI-H1975 tumor cells+TALL-104 effector cells (no IGN004). Plates were set up with duplicate samples.

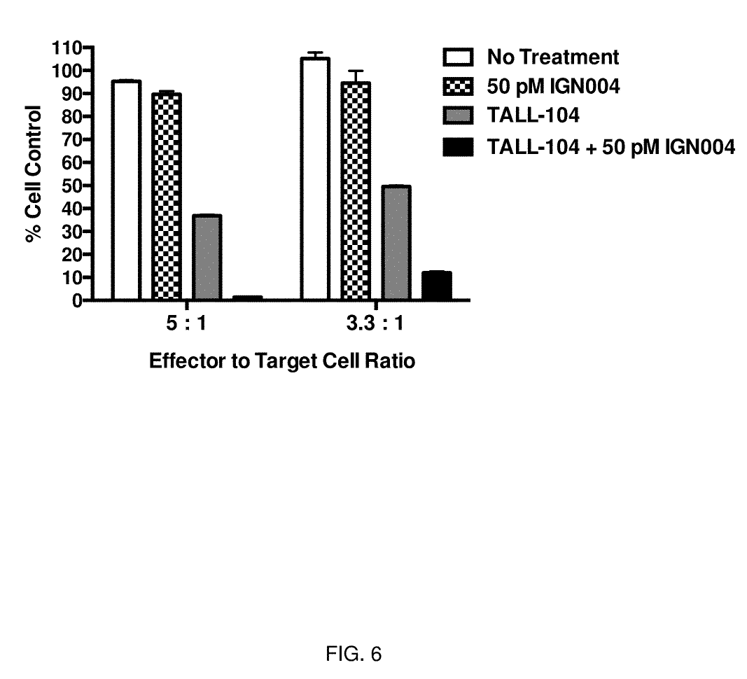

As depicted in FIG. 6, IGN004 treatment caused a small decrease in the viability of the A549 tumor cells (5.7% and 10.6%). TALL-104 effector cells demonstrated significant killing in the absence of IGN004 and both 5:1 and 3.3:1 E:T ratios (58.6% and 55.7%, respectively). However, the combination of 50 pM IGN004 and TALL-104 cells lead to much more effective killing of the NCI-H1975 tumor cell targets at both E:T ratios (93.8% and 93.2%, respectively). This effect was stronger than the combination of either agent alone, leading to the conclusion that IGN004 and TALL-104 can have a synergistic effect upon NCI-H1975 tumor cells leading to much more robust tumor cell killing.

### Example 8

In this example, the potency of the TALL-104 tumor cell killing enhancement by IGN004 was assessed using NCI-H1975 NSCLC tumor cells.

Co-cultures were set up in 24-well plates as described in Examples 6 and 7 using NCI-H1975 tumor cells as targets and TALL-104 cells as effectors, after incubating the tumor cells with the indicated concentration of IGN004 for 3 hours. Unbound IGN004 was washed away prior to adding effector cells to achieve an E:T ratio of 3.3:1. Incubation time for the co-cultures was 48 hours at 37° C.

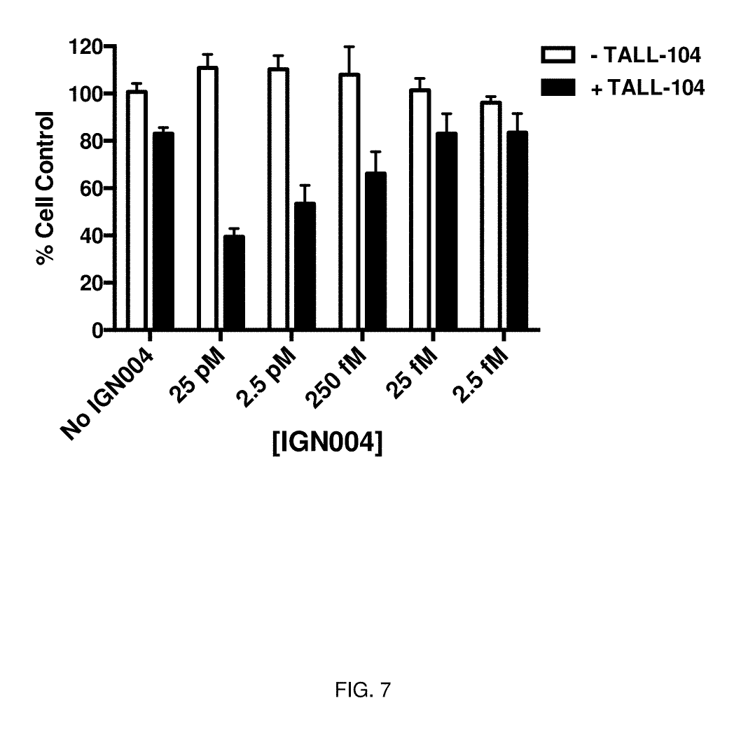

As depicted in FIG. 7, TALL-104 effector cells killed 17% of the NCI-H1975 tumor cells in the absence of IGN004 co-treatment. Treatment with IGN004 in combination with TALL-104 cells at concentrations from 0.25 to 25 pM caused an increase in tumor cell killing, compared to TALL-104 treatment alone. This result demonstrates that the enhancement in immune cell killing is a very potent effect and can occur at very low concentrations of drug.

### Example 9

In this example, the tumor cell killing activity of downregulated TALL-104 effector cells was assessed on A549 NSCLC tumor cells in the presence or absence of 10 pM IGN004 at different E:T ratios.

Co-cultures were set up in 24-well plates as described in Examples 6 and 7 using A549 tumor cells as targets and TALL-104 cells as effectors, after incubating the tumor cells with 10 pM IGN004 for 3 hours. Unbound IGN004 was washed away prior to adding effector cells to achieve an E:T ratio of 3:1, 1.5:1, or 0.75:1. The TALL-104 cells were washed and IL-2 removed from the media 2 days prior to the assay setup in an effort to reduce their activation status and killing activity. Incubation time for the co-cultures was 5 days at 37° C.

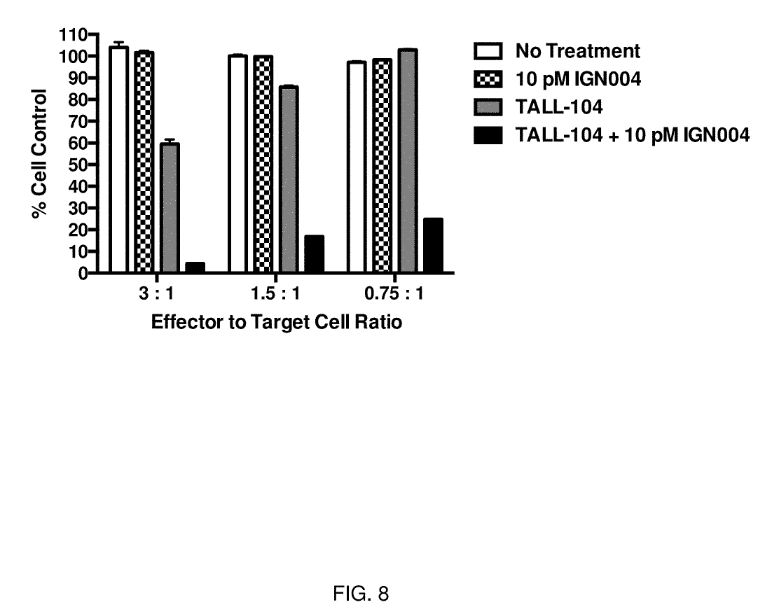

As depicted in FIG. 8, 10 pM IGN004 alone had no effect on the tumor cells. At the 3:1 E:T ratio TALL-104 cells killed approximately 40% of the A549 tumor cells in the absence of drug but at lower E:T ratios the effector cells were ineffective at tumor cell killing. In the presence of 10 pM IGN004 the TALL-104 cells demonstrated robust tumor cell killing, even at 0.75:1 E:T where TALL-104 had no effect on the tumor cells without drug. These results demonstrate that IGN004 is able to reverse the downregulation in killing activity of TALL-104 effector cells achieved by IL-2 starvation.

### Example 10

In this example, the tumor cell killing activity of TALL-104 effector cells was assessed in the presence or absence of IGN004 or IGN004 non-fused mAb.

Co-cultures were set up in 24-well plates as described in Examples 6 and 7 using A549 tumor cells as targets and TALL-104 cells as effectors, after incubating the tumor cells with 10 pM of either IGN004 or IGN004 non-fused mAb for 3 hours. Unbound protein was washed away prior to adding effector cells to achieve an E:T ratio of 1.5:1, 0.75:1, or 0.375:1. The TALL-104 cells were washed and IL-2 removed from the media 2 days prior to the assay setup. Incubation time for the co-cultures was 4 days at 37° C.

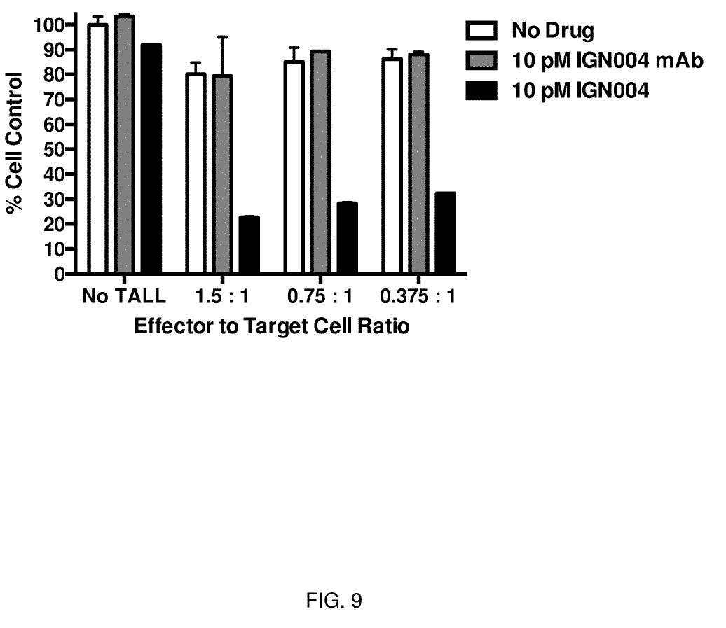

As depicted in FIG. 9, 10 pM IGN004 non-fused mAb alone had no effect on the tumor cells and 10 pM IGN004 had only a slight effect (<10%). At all E:T ratios TALL-104 cells demonstrated a low level of tumor cell killing in the absence of drug. In the presence of 10 pM IGN004 mAb, the TALL-104 cells killed at an equivalent rate to TALL-104 cells without drug. However, with 10 pM IGN004 there was a significant increase in the tumor cell killing by TALL-104 cells, compared to no drug (70-80% vs. 10-20% killing). These results demonstrate that the antibody portion of the IGN004 is not solely responsible for the synergistic effects on TALL-104-mediated tumor cell killing.

### Example 11

In this example, the tumor cell killing activity of TALL-104 effector cells was assessed in the presence or absence of IGN004, a control TAA Ab-IFN-α fusion protein, or the combination of IGN004 non-fused mAb+non-fused IFN-α.

Co-cultures were set up in 24-well plates as described in Examples 6 and 7 using A549 tumor cells as targets and TALL-104 cells as effectors, after incubating the tumor cells with 10 pM of either IGN004, control antibody-IFN-α fusion protein, or the combination of IGN004 non-fused mAb+non-fused IFN-α2b for 3 hours. Effector cells were then added without washing away treatment protein to achieve an E:T ratio of 1:1 or 1.5:1. The TALL-104 cells were washed and IL-2 removed from the media 2 days prior to the assay setup. Incubation time for the co-cultures was 5 days at 37° C.

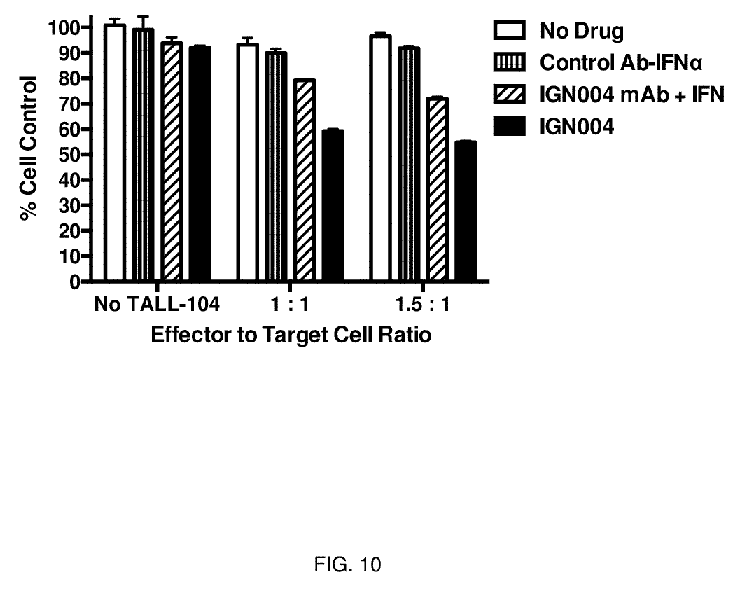

As depicted in FIG. 10, 10 pM control antibody-IFN-α2b alone had no effect on the tumor cells. 10 pM IGN004 or the combination of IGN004 non-fused mAb and non-fused IFN-α2b had only a slight effect (<10%). At both E:T ratios TALL-104 cells demonstrated a low level of tumor cell killing in the absence of drug (<10%). In the presence of 10 pM control antibody-IFN-α fusion the TALL-104 cells killed at an equivalent rate to TALL-104 cells without drug. With 10 pM of the combination of IGN004 mAb+non-fused IFN-α2b the TALL-104 effector cells killed more A549 tumor cells (14% and 25% increase in killing at 1:1 and 1.5:1 E:T, respectively). However, with 10 pM IGN004 there was a much higher increase in the tumor cell killing by TALL-104 cells, compared to no drug (34% and 42% increase in killing at 1:1 and 1.5:1, respectively). These results demonstrate that the TAA Ab-IFN-α fusion protein must bind to the tumor cell to exert its function of enhancing immune cell killing of tumor cells in the tumor microenvironment, and that the TAA Ab and IFN must be fused together to have the complete effect. Therefore, the enhancement in immune cell function should only occur at sites where the antibody target antigen is expressed.

### Example 12

In this example, the tumor cell killing activity of the NK effector cell line NK-92 (ATCC CRL-2407) was assessed in the presence or absence of IGN004 or a control TAA-Ab-IFN-α fusion protein at two E:T ratios using the OVCAR-3 ovarian cancer cell line (ATCC HTB-161).

The NK-92 tumor cell killing assay was performed similarly to the TALL-104 killing assays described in Examples 6 and 7. Co-cultures were set up in 24-well plates using OVCAR-3 tumor cells as targets and NK-92 cells as effectors, after incubating the tumor cells with 10 pM of either IGN004 or control TAA Ab-IFN-α fusion protein for 3 hours. Effector cells were then added without washing away treatment protein to achieve an E:T ratio of 1.5:1 or 0.5:1. The NK-92 cells were washed and IL-2 removed from the media 1 day prior to the assay setup. Incubation time for the co-cultures was 2 days at 37° C.

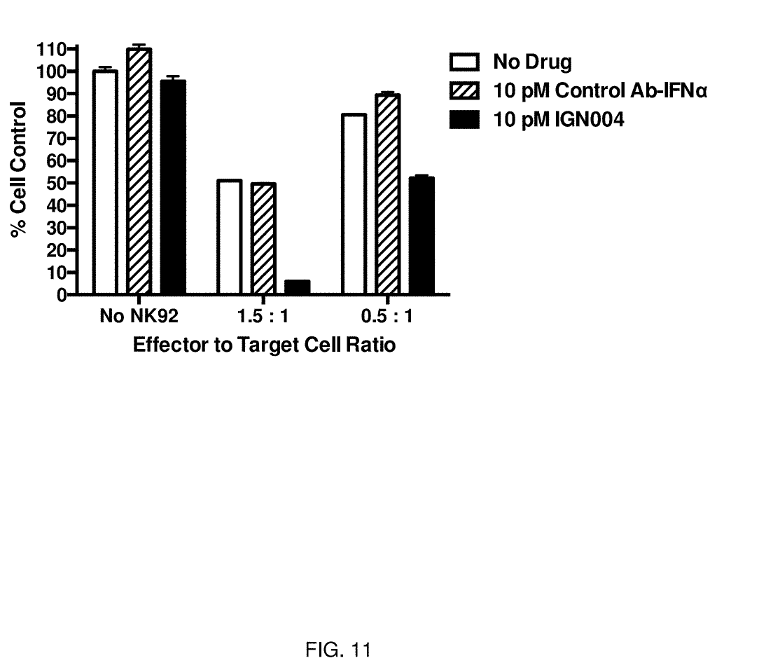

As depicted in FIG. 11, 10 pM of either treatment protein had no effect on the tumor cells in the absence of effector cells. NK-92 effector cells demonstrated robust killing of tumor cells in the absence of drug at 1.5:1 E:T ratio (49% killing) and modest killing at 0.5:1 (19% killing). In the presence of 10 pM control TAA Ab-IFN-α fusion the NK-92 cells killed at an equivalent rate to effector cells without drug. With 10 pM IGN004 there was a significant increase in the tumor cell killing by NK-92 cells, compared to no drug (45% and 29% increase in killing at 1.5:1 and 0.5:1, respectively). These results demonstrate that IGN004 is able to enhance the NK cell mediated killing of tumor cells, and that the TAA Ab-IFN fusion protein must bind to the tumor cell to exert this function. Therefore, the enhancement in immune cell function should only occur at sites where the antibody target antigen is expressed.

### Example 13

In this example, the tumor cell killing activity of the NK-92 effector cells was assessed in the presence or absence of IGN004 or non-fused IFN-α2b at two E:T ratios using NCI-H1975 NSCLC tumor cells.

The NK-92 tumor cell killing assay was performed as described in Example 12. Co-cultures were set up in 24-well plates using NCI-H1975 cells as targets and NK-92 cells as effectors, after incubating the tumor cells with 10 pM IGN004 or 100 pM non-fused IFN-α2b for 3 hours. Effector cells were then added without washing away treatment protein to achieve an E:T ratio of 1:1 or 0.3:1. The NK-92 cells were washed and IL-2 removed from the media 1 day prior to the assay setup. Incubation time for the co-cultures was 4 days at 37° C.

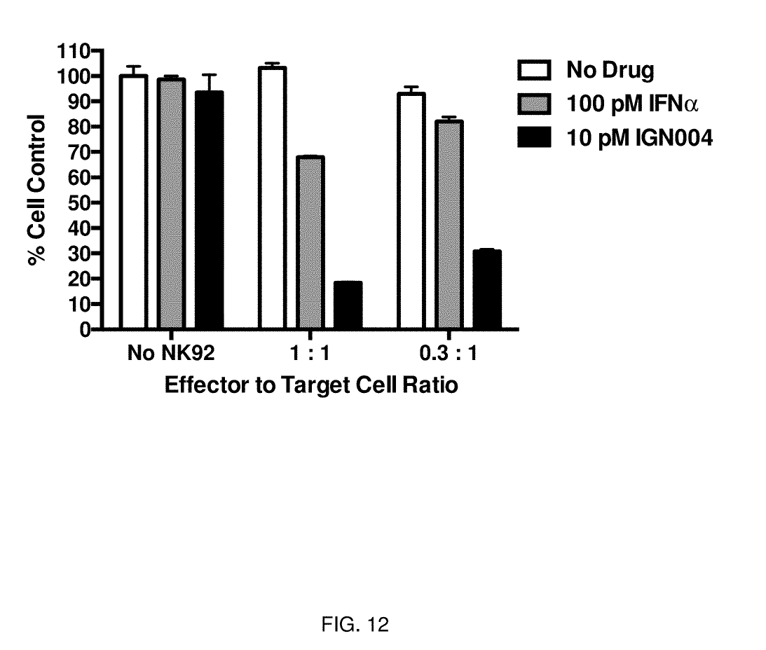

As depicted in FIG. 12, treatment with either protein had no effect on the tumor cells in the absence of effector cells. NK-92 effector cells demonstrated little to no killing of tumor cells in the absence of drug. In the presence of 100 pM non-fused IFN-α2b the NK-92 cells killed more tumor cells than NK-92 cells in the absence of drug. With 10 pM IGN004 there was a significant increase in the tumor cell killing by NK-92 cells, compared to no drug (85% and 62% increase in killing at 1:1 and 0.3:1, respectively) and non-fused IFN-α2b (50% and 51% increase in killing at 1:1 and 0.3:1, respectively). These results demonstrate that IGN004 is able to enhance the NK cell mediated killing of tumor cells, and that the TAA Ab-IFN fusion protein mediates this effect much better than non-fused IFN-α2b demonstrating the importance of targeting of the IFN to the tumor cell surface via TAA antibody.

### Example 14

In this example, GRP94-positive A1847 ovarian cancer cells were seeded into 96-well plates and incubated for 20 hours at 37° C. to allow for adherence. After incubation, tumor cells were treated for 4 hours with 50 pM IGN004 or 50 pM of a control TAA Ab-IFNα fusion molecule. After 4 hours, anti-mesothelin CAR-T cells were added at an effector to target ratio of 2:1. Co-cultures were incubated for a further 72 hours and cell viability was monitored in real time using the xCELLigence RTCA system (Acea Biosciences).

The anti-mesothelin CAR-T effector cells at the sub-optimal E:T ratio of 2:1 caused a reduction in A1847 tumor cell viability throughout the experiment, compared to tumor cells alone. The addition of a control TAA Ab-IFNα fusion molecule did not enhance the CAR-T killing of the tumor cell targets. In contrast, IGN004 at 50 pM enhanced the CAR-T killing of A1847 tumor cells over time, resulting in a decreased cell index compared to A1847+CAR-T.

### Sequence Listings

The amino acid sequences listed in the accompanying sequence listing are shown using standard three letter code for amino acids, as defined in 37 C.F.R. 1.822.

SEQ ID NO: 1 is the amino acid sequence of a human wildtype IFN-α2b molecule.

SEQ ID NO: 2 is the amino acid sequence of an IFN-α2b mutant molecule.

SEQ ID NO: 3 is the amino acid sequence of a wildtype IFN-α14 molecule.

SEQ ID NO: 4 is the amino acid sequence of a wildtype IFN-β-1a molecule.

SEQ ID NO: 5 is the amino acid sequence of a wildtype IFN-β-1b molecule.

SEQ ID NO: 6 is the amino acid sequence encoding the heavy chain of an anti-HER2/neu antibody. SEQ ID NO: 7 is the amino acid sequence encoding the light chain of an anti-HER2/neu antibody.

SEQ ID NO: 8 is the amino acid sequence encoding the heavy chain of an anti-CD20 antibody. SEQ ID NO: 9 is the amino acid sequence encoding the light chain of an anti-CD20 antibody.

SEQ ID NO: 10 is the amino acid sequence encoding the heavy chain of an anti-CD138 antibody. SEQ ID NO: 11 is the amino acid sequence encoding the light chain of an anti-CD138 antibody.

SEQ ID NO: 12 is the amino acid sequence encoding the heavy chain of an anti-GRP94 antibody. SEQ ID NO: 13 is the amino acid sequence encoding the light chain of an anti-GRP94 antibody.

SEQ ID NO: 14 is the amino acid sequence encoding the heavy chain of an anti-CD33 antibody. SEQ ID NO: 15 is the amino acid sequence encoding the light chain of an anti-CD33 antibody.

SEQ ID NO: 16 is the amino acid sequence encoding the heavy chain variable region of an anti-CD70 antibody. SEQ ID NO: 17 is the amino acid sequence encoding the light chain of an anti-CD70 antibody.

SEQ ID NOs: 18-28 are the amino acid sequences of various peptide linkers.

SEQUENCE LISTINGS
- Amino acid sequence of a human wildtype IFN-α2b molecule.
SEQ ID NO: 1
CDLPQTHSLGSRRTLMLLAQMRRISLFSCLKDRHDFGFPQEEFGNQFQKAETIPVLHEMIQQIF
NLFSTKDSSAAWDETLLDKFYTELYQQLNDLEACVIQGVGVTETPLMKEDSILAVRKYFQRITLY
LKEKKYSPCAWEVVRAEIMRSFSLSTNLQESLRSKE
- Amino acid sequence of an IFN-α2b mutant molecule.
SEQ ID NO: 2
CDLPQTHSLGSRRTLMLLAQMRRISLFSCLKDRHDFGFPQEEFGNQFQKAETIPVLHEMIQQIF
NLFSTKDSSAAWDETLLDKFYTELYQQLNDLEACVIQGVGVTETPLMKEDSILAVRKYFQRITLY
LKEKKYSPCAWEVVRAEIMASFSLSTNLQESLASKE
- Amino acid sequence of a wildtype IFN-α14 molecule.
SEQ ID NO: 3
CNLSQTHSLNNRRTLMLMAQMRRISPFSCLKDRHDFEFPQEEFDGNQFQKAQAISVLHEMMQ
QTFNLFSTKNSSAAWDETLLEKFYIELFQQMNDLEACVIQEVGVEETPLMNEDSILAVKKYFQRI
TLYLMEKKYSPCAWEVVRAEIMRSLSFSTNLQKRLRRKD
- Amino acid sequence of a wildtype IFN-β-1a molecule.
SEQ ID NO: 4
MSYNLLGFLQRSSNFQCQKLLWQLNGRLEYCLKDRMNFDIPEEIKQLQQFQKEDAALTIYEML
QNIFAIFRQDSSSTGWNETIVENLLANVYHQINHLKTVLEEKLEKEDFTRGKLMSSLHLKRYYGR
ILHYLKAKEYSHCAWTIVRVEILRNFYFINRLTGYLRN
- Amino acid sequence of a wildtype IFN-β-1b molecule.
SEQ ID NO: 5
MSYNLLGFLQRSSNFQSQKLLWQLNGRLEYCLKDRMNFDIPEEIKQLQQFQKEDAALTIYEML
QNIFAIFRQDSSSTGWNETIVENLLANVYHQINHLKTVLEEKLEKEDFTRGKLMSSLHLKRYYGR
ILHYLKAKEYSHCAWTIVRVEILRNFYFINRLTGYLRN
- Amino acid sequence encoding the heavy chain of an
anti-HER2/neu antibody.
SEQ ID NO: 6
EVQLVESGGGLVQPGGSLRLSCAASGFNIKDTYIHWVRQAPGKGLEWVARIYPTNGYTRYADS
VKGRFTISADTSKNTAYLQMNSLRAEDTAVYYCSRWGGDGFYAMDYWGQGTLVTVSSASTK
GPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQ
KSLSLSPGK
- Amino acid sequence encoding the light chain of an
anti-HER2/neu antibody.
SEQ ID NO: 7
DIQMTQSPSSLSASVGDRVTITCRASQDVNTAVAWYQQKPGKAPKLLIYSASFLYSGVPSRFS
GSRSGTDFTLTISSLQPEDFATYYCQQHYTTPPTFGQGTKVEIKRTVAAPSVFIFEPSDEQLKS
GTASVVCLLNNFYPREAKVQWKVDNALQSGNSQESVTEQDSKDSTYSLSSTLTLSKADYEKHKVY
ACEVTHQGLSSPVTKSFNRGEC
- Amino acid sequence encoding the heavy chain of an
anti-CD20 antibody.
SEQ ID NO: 8
QVQLQQPGAELVKPGASVKMSCKASGYTFTSYNMHWVKQTPGRGLEWIGAIYPGNGDTSYN
QKFKGKATLTADKSSSTAYMQLSSLTSEDSAVYYCARSTYYGGDWYFNVWGAGTTVTVSAAS
TKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHEALHNHY
TQKSLSLSPGK
- Amino acid sequence encoding the light chain of an
anti-CD20 antibody.
SEQ ID NO: 9
QIVLSQSPAILSASPGEKVTMTCRASSSVSYIHWFQQKPGSSPKPWIYATSNLASGVPVRFSGS
GSGTSYSLTISRVEAEDAATYYCQQWTSNPPTFGGGTKLEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC
- Amino acid sequence encoding the heavy chain of an
anti-CD138 antibody.
SEQ ID NO: 10
QVQLQQSGSELMMPGASVKISCKATGYTFSNYWIEWVKQRPGHGLEWIGEILPGTGRTIYNEK
FKGKATFTADISSNTVQMQLSSLTSEDSAVYYCARRDYYGNFYYAMDYWGQGTSVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYT
QKSLSLSPGK
- Amino acid sequence encoding the light chain of an
anti-CD138 antibody.
SEQ ID NO: 11
DIQMTQSTSSLSASLGDRVTISCSASQGINNYLNWYQQKPDGTVELLIYYTSTLQSGVPSRFSG
SGSGTDYSLTISNLEPEDIGTYYCQQYSKLPRTFGGGTKLEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC
- Amino acid sequence encoding the heavy chain of an
anti-GRP94 antibody.
SEQ ID NO: 12
QVQLVQSGAEVKKPGASVKVSCKASGYTFTSYAMHWVRQAPGQRLEWMGWINAGNGNTKY
SQKFQGRVTITRDTSASTAYMELSSLRSEDTAVYYCARAHFDYWGQGTLVTVSAASTKGPSVF
PLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSL
SLSPGK
- Amino acid sequence encoding the light chain of an
anti-GRP94 antibody.
SEQ ID NO: 13
EIELTQSPSSLSASVGDRVTITCRASQSISSYLNWYQQKPGKAPKLLIYAASSLQSGVPSRFSGS
GSGTDFTLTISSLQPEDFATYYCQQSYSTPPTFGQGTKVEIKRTVAAPSVFIFPPSDEQLKSGTA
SVVCLLNNFYPREAKVQWKVDNALQSGNSQESVTEQDSKDSTYSLSSTLTLSKADYEKHKVYA
CEVTHQGLSSPVTKSFNRGEC
- Amino acid sequence encoding the heavy chain of an
anti-CD33 antibody.
SEQ ID NO: 14
QVQLVQSGAEVKKPGSSVKVSCKASGYTITDSNIHWVRQAPGQSLEWIGYIYPYNGGTDYNQKF
KNRATLTVDNFINTAYMELSSLRSEDTAFYYCVNGNPWLAYWGQGTLVTVSSASTKGPSVFPL
APSSKSTSGGTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLSPG
K
- Amino acid sequence encoding the light chain of an
anti-CD33 antibody.
SEQ ID NO: 15
DIQLTQSPSTLSASVGDRVTITCRASESLDNYGIRFLTWFQQKPGKAPKLLMYAASNQGSGVPS
RFSGSGSGTEFTLTISSLQPDDFATYYCQQTKEVPWSFGQGTKVEVKRTVAAPSVFIFPPSDE
QLKSGTASVVCLLNNFYPREAKVQWKVDNALQSGNSQESVTEQDSKDSTYSLSSTLTLSKADYEK
HKVYACEVTHQGLSSPVTKSFNRGEC
- Amino acid sequence encoding the heavy chain of an
anti-CD70 antibody.
SEQ ID NO: 16
QVQLVESGGGVVQPGRSLRLSCAASGFTFSSYIMHWVRQAPGKGLEWVAVISYDGRNKYYAD
SVKGRFTISRDNSKNTLYLQMNSLRAEDTAVYYCARDTDGYDFDYWGQGTLVTVSS
- Amino acid sequence encoding the light chain of an
anti-CD70 antibody.
SEQ ID NO: 17
EIVLTQSPATLSLSPGERATLSCRASQSVSSYLAWYQQKPGQAPRLLIYDASNRATGIPARFSG
SGSGTDFTLTISSLEPEDFAVYYCQQRTNWPLTFGGGTKVEIK
- Amino acid sequence of a peptide linker.
SEQ ID NO: 18
SGGGGS
- Amino acid sequence of a peptide linker.
SEQ ID NO: 19
AEAAAKEAAAKAGS
- Amino acid sequence of a peptide linker.
SEQ ID NO: 20
GGGGS
- Amino acid sequence of a peptide linker.
SEQ ID NO: 21
SGGGGSGGGGS
- Amino acid sequence of a peptide linker.
SEQ ID NO: 22
GGGGG
- Amino acid sequence of a peptide linker.
SEQ ID NO: 23
GAGAGAGAGA
- Amino acid sequence of a peptide linker.
SEQ ID NO: 24
AEAAAKAGS
- Amino acid sequence of a peptide linker.
SEQ ID NO: 25
GGGGGGGG
- Amino acid sequence of a peptide linker.
SEQ ID NO: 26
AEAAAKEAAAKA
- Amino acid sequence of a peptide linker.
SEQ ID NO: 27
AEAAAKA
- Amino acid sequence of a peptide linker.
SEQ ID NO: 28
GGAGG

1-39. (canceled)

40. A method of treating a proliferative disease in an individual, comprising administering to the individual a therapeutically effective amount of an isolated non-naturally occurring tumor associated antigen antibody-interferon (“TAA Ab-IFN”) fusion molecule, wherein the TAA-IFN-α fusion molecule is administered to the individual at a weekly dosage selected from the group consisting of no greater than 0.0001 mg/kg, no greater than 0.0003 mg/kg, no greater than 0.001 mg/kg, no greater than 0.003 mg/kg, no greater than 0.01 mg/kg, no greater than 0.03 mg/kg, no greater than 0.1 mg/kg, no greater than 0.2 mg/kg, no greater than 0.3 mg/kg, no greater than 0.4 mg/kg, no greater than 0.5 mg/kg, no greater than 0.6 mg/kg, no greater than 0.7 mg/kg, no greater than 0.8 mg/kg, and no greater than 0.9 mg/kg.

41. The method according to claim 40, wherein the TAA Ab-IFN fusion molecule is administered to the individual at a weekly dosage selected from the group consisting of about 0.0001 to about 0.0003 mg/kg, about 0.0003 to about 0.001 mg/kg, about 0.001 to about 0.003 mg/kg, about 0.003 to about 0.01 mg/kg, about 0.01 to about 0.03 mg/kg, about 0.03 to about 0.1 mg/kg, about 0.1 to about 0.3 mg/kg, about 0.3 to about 0.4 mg/kg, about 0.4 to about 0.5 mg/kg, about 0.5 to about 0.6 mg/kg, about 0.6 to about 0.7 mg/kg, about 0.7 to about 0.8 mg/kg, and about 0.8 to about 0.9 mg/kg.

42. The method according to claim 40, wherein the TAA Ab-IFN fusion molecule is administered to the individual at a weekly dosage of about 0.003 to about 0.01 mg/kg.

43. The method according to claim 40, wherein the TAA Ab-IFN fusion molecule is administered to the individual at a weekly dosage of about 0.01 to about 0.03 mg/kg.

44. The method according to claim 40, wherein the TAA Ab-IFN fusion molecule is administered to the individual at a weekly dosage of about 0.03 to about 0.1 mg/kg.

45. The method according to claim 40, wherein the proliferative disease is a cancer is selected from the group consisting of: B cell lymphoma; a lung cancer (small cell lung cancer and non-small cell lung cancer); a bronchus cancer; a colorectal cancer; a prostate cancer; a breast cancer; a pancreas cancer; a stomach cancer; an ovarian cancer; a urinary bladder cancer; a brain or central nervous system cancer; a peripheral nervous system cancer; an esophageal cancer; a cervical cancer; a melanoma; a uterine or endometrial cancer; a cancer of the oral cavity or pharynx; a liver cancer; a kidney cancer; a biliary tract cancer; a small bowel or appendix cancer; a salivary gland cancer; a thyroid gland cancer; a adrenal gland cancer; an osteosarcoma; a chondrosarcoma; a liposarcoma; a testes cancer; and a malignant fibrous histiocytoma; a skin cancer; a head and neck cancer; lymphomas; sarcomas; multiple myeloma; and leukemias.

46. The method according to claim 45, wherein the individual has a recurrent cancer.

47. The method according to claim 45, wherein the individual has resistant or refractory cancer.

48. The method according to claim 40, wherein the TAA Ab-IFN fusion molecule comprises a fully human monoclonal antibody selected from the group consisting of a fully human anti-HER2/neu Ab, a fully human anti-CD20 Ab, a fully human anti-CD138 Ab, a fully human anti-GRP94 (endoplasmin) Ab, a fully human anti-CD33 Ab, and a fully human anti-CD70 Ab attached to a type 1 interferon molecule selected from the group consisting of an interferon (IFN)-α molecule, an IFN-α mutant molecule, an IFN-β-1a molecule, an IFN-β-1b molecule, and an IFN-β mutant molecule.

49. The method according to claim 48, wherein the type 1 interferon is selected from the group consisting of a human IFN-α2b molecule having the amino acid sequence of SEQ ID NO: 1, a human IFN-α2b mutant molecule having the amino acid sequence of SEQ ID NO: 2, a human IFN-α14 molecule having the amino acid sequence of SEQ ID NO: 3, a human IFN-β-1a molecule having the amino acid sequence of SEQ ID NO: 4, and a human IFN-β-1b molecule having the amino acid sequence of SEQ ID NO: 5.

50. A method according to claim 40, wherein the proliferative disease is a cancer selected from the group consisting of breast cancer, ovarian cancer and non-small cell lung cancer (NSCLC), and wherein the TAA Ab-IFN fusion molecule is an anti-HER2/neu-IFN-α fusion molecule.

51. A method according to claim 40, wherein the proliferative disease is a cancer selected from the group consisting of B-cell Non-Hodgkin's lymphoma (NHL) and B-cell chronic lymphocytic leukemia (CLL), and wherein the TAA Ab-IFN fusion molecule is an anti-CD20-IFN-α fusion molecule.

52. A method according to claim 40, wherein the proliferative disease is a cancer selected from the group consisting of multiple myeloma, breast cancer, and bladder cancer, and wherein the TAA Ab-IFN fusion molecule is an anti-CD138-IFN-α fusion molecule.

53. A method according to claim 40, wherein the proliferative disease is a cancer selected from the group consisting of NSCLC, acute myeloid leukemia (AML), multiple myeloma, melanoma, and pancreatic cancer, and wherein the TAA Ab-IFN fusion molecule is an anti-GRP94-IFN-α fusion molecule.

54. A method according to claim 40, wherein the proliferative disease is a cancer selected from the group consisting of AML, chronic myeloid leukemia (CML) and multiple myeloma, and wherein the TAA Ab-IFN fusion molecule is an anti-CD33-IFN-α fusion molecule.

55. A method according to claim 40, wherein the proliferative disease is a cancer selected from the group consisting of renal cell carcinoma (RCC), Waldenstrom macroglobulinemia, multiple myeloma, and NHL, and wherein the TAA Ab-IFN fusion molecule is an anti-CD70-IFN-α fusion molecule.

56. A method of treating a proliferative disease in an individual, comprising administering to the individual: a) a therapeutically effective amount of an isolated non-naturally occurring tumor associated antigen antibody-interferon (“TAA Ab-IFN”) fusion molecule, and b) immunotherapy, wherein the TAA-IFN-α fusion molecule is administered to the individual at a weekly dosage selected from the group consisting of no greater than 0.0001 mg/kg, no greater than 0.0003 mg/kg, no greater than 0.001 mg/kg, no greater than 0.003 mg/kg, no greater than 0.01 mg/kg, no greater than 0.03 mg/kg, no greater than 0.1 mg/kg, no greater than 0.2 mg/kg, no greater than 0.3 mg/kg, no greater than 0.4 mg/kg, no greater than 0.5 mg/kg, no greater than 0.6 mg/kg, no greater than 0.7 mg/kg, no greater than 0.8 mg/kg, and no greater than 0.9 mg/kg.

57. The methods according to claim 56, wherein the immunotherapy is selected from the group consisting of: treatment using agonistic, antagonistic, or blocking antibodies to co-stimulatory or co-inhibitory molecules (immune checkpoints); treatment using bispecific T cell engaging antibodies (BiTE®); treatment involving administration of biological response modifiers; treatment using therapeutic vaccines; treatment using dendritic cell vaccines; treatment using tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells; treatment using TALL-104 cells; and treatment using immunostimulatory agents such as Toll-like receptor (TLR) agonists.

58. The method according to claim 56, wherein the cancer is selected from the group consisting of breast cancer, ovarian cancer and non-small cell lung cancer (NSCLC); wherein the immunotherapy is selected from the group consisting of: treatment using agonistic, antagonistic, or blocking antibodies to co-stimulatory or co-inhibitory molecules (immune checkpoints); treatment using chimeric antigen receptor (CAR)-T cells; treatment using CAR-NK cells; and treatment using bispecific T cell engaging antibodies (BiTE®); and wherein the TAA Ab-IFN fusion molecule is an anti-HER2/neu-IFN-α fusion molecule.

59. The method according to claim 56, wherein the cancer is selected from the group consisting of B-cell Non-Hodgkin's lymphoma (NHL) and B-cell chronic lymphocytic leukemia (CLL); wherein the immunotherapy is selected from the group consisting of: treatment using agonistic, antagonistic, or blocking antibodies to co-stimulatory or co-inhibitory molecules (immune checkpoints); treatment using chimeric antigen receptor (CAR)-T cells; treatment using CAR-NK cells; and treatment using bispecific T cell engaging antibodies (BiTE®); and wherein the TAA Ab-IFN fusion molecule is an anti-CD20-IFN-α fusion molecule.

60. The method according to claim 56, wherein the cancer is selected from the group consisting of multiple myeloma, breast cancer, and bladder cancer; wherein the immunotherapy is selected from the group consisting of: treatment using agonistic, antagonistic, or blocking antibodies to co-stimulatory or co-inhibitory molecules (immune checkpoints); treatment using chimeric antigen receptor (CAR)-T cells; treatment using CAR-NK cells; and treatment using bispecific T cell engaging antibodies (BiTE®); and wherein the TAA Ab-IFN fusion molecule is an anti-CD138-IFN-α fusion molecule.

61. The method according to claim 56, wherein the cancer is selected from the group consisting of NSCLC, acute myeloid leukemia (AML), multiple myeloma, melanoma, and pancreatic cancer; wherein the immunotherapy is selected from the group consisting of: treatment using agonistic, antagonistic, or blocking antibodies to co-stimulatory or co-inhibitory molecules (immune checkpoints); treatment using chimeric antigen receptor (CAR)-T cells; treatment using CAR-NK cells; and treatment using bispecific T cell engaging antibodies (BiTE®); and wherein the TAA Ab-IFN fusion molecule is an anti-GRP94 Ab-IFN-α fusion molecule.

62. The method according to claim 56, wherein the cancer is selected from the group consisting of AML, chronic myeloid leukemia (CML) and multiple myeloma; wherein the immunotherapy is selected from the group consisting of: treatment using agonistic, antagonistic, or blocking antibodies to co-stimulatory or co-inhibitory molecules (immune checkpoints); treatment using chimeric antigen receptor (CAR)-T cells; treatment using CAR-NK cells; and treatment using bispecific T cell engaging antibodies (BiTE®); and wherein the TAA Ab-IFN fusion molecule is an anti-CD33-IFN-α fusion molecule.

63. The method according to claim 56, wherein the cancer is selected from the group consisting of renal cell carcinoma (RCC), Waldenstrom macroglobulinemia, multiple myeloma, and NHL; wherein the immunotherapy is selected from the group consisting of: treatment using agonistic, antagonistic, or blocking antibodies to co-stimulatory or co-inhibitory molecules (immune checkpoints); treatment using chimeric antigen receptor (CAR)-T cells; treatment using CAR-NK cells; and treatment using bispecific T cell engaging antibodies (BiTE®); and wherein the TAA Ab-IFN fusion molecule is an anti-CD70-IFN-α fusion molecule.

64. The method according to claim 56, wherein the combination therapy methods comprise administering the TAA Ab-IFN fusion molecule and immunotherapy simultaneously.

65. The method according to claim 56, wherein the administration of the TAA Ab-IFN fusion molecule and immunotherapy are concurrent.

66. The method according to claim 56, wherein the administration of the TAA Ab-IFN fusion molecule and immunotherapy are non-concurrent.


SEQUENCE LISTING (28 sequences)

SEQ ID NO: 1 - protein, 165 aa, Homo sapiens [sequence omitted]

SEQ ID NO: 2 - protein, 165 aa, Artificial
  IFNa2b Mutant Molecule
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMASFSLSTNLQESLASKE

SEQ ID NO: 3 - protein, 166 aa, Homo sapiens
CNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 4 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 5 - protein, 166 aa, Homo sapiens
MSYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 6 - protein, 450 aa, Artificial
  HC of an anti-HER2/neu antibody
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 214 aa, Artificial
  LC of an anti-HER2/neu antibody
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFEPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, Artificial
  HC of an anti-CD20 antibody
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 213 aa, Artificial
  LC of an anti-CD20 antibody
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 452 aa, Artificial
  HC of an anti-CD138 antibody
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Artificial
  LC of an anti-CD138 antibody
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 444 aa, Artificial
  HC of an anti-GRP94 antibody
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARAHFDYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, Artificial
  LC of an anti-GRP94 antibody
EIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 446 aa, Artificial
  HC of an anti-CD33 antibody
QVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 218 aa, Artificial
  LC of an anti-CD33 antibody
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 118 aa, Artificial
  HCVR of an anti-CD70 antibody
QVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTVSS

SEQ ID NO: 17 - protein, 107 aa, Artificial
  LCVR of an anti-CD70 antibody
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 18 - protein, 6 aa, Artificial
  Peptide linker
SGGGGS

SEQ ID NO: 19 - protein, 14 aa, Artificial
  Peptide linker
AEAAAKEAAAKAGS

SEQ ID NO: 20 - protein, 5 aa, Artificial
  Peptide linker
GGGGS

SEQ ID NO: 21 - protein, 11 aa, Artificial
  Peptide linker
SGGGGSGGGGS

SEQ ID NO: 22 - protein, 5 aa, Artificial
  Peptide linker
GGGGG

SEQ ID NO: 23 - protein, 10 aa, Artificial
  Peptide linker
GAGAGAGAGA

SEQ ID NO: 24 - protein, 9 aa, Artificial
  Peptide linker
AEAAAKAGS

SEQ ID NO: 25 - protein, 8 aa, Artificial
  Peptide linker
GGGGGGGG

SEQ ID NO: 26 - protein, 12 aa, Artificial
  Peptide linker
AEAAAKEAAAKA

SEQ ID NO: 27 - protein, 7 aa, Artificial
  Peptide linker
AEAAAKA

SEQ ID NO: 28 - protein, 5 aa, Artificial
  Peptide linker
GGAGG